SEQUENCE LISTING (69 sequences)

SEQ ID NO: 1 - protein, 161 aa, Herpes simplex virus type 1
MSQTQPPAPVGPGDPDVYLKGVPSAGMHPRGVHAPRGHPRMISGPPQRGDNDQAAGQCGDSGLLRVGADTTISKPSEAVRPPTIPRTPRVPREPRVPRPPREPREPRVPRAPRDPRVPRDPRDPRQPRSPREPRSPREPRSPREPRTPRTPREPRTARGSV

SEQ ID NO: 2 - protein, 69 aa, Herpes simplex virus type 1
MPRVPRPPREPREPRVPRAPRDPRVPRDPRDPRQPRSPREPRSPREPRSPREPRTPRTPREPRTARGSV

SEQ ID NO: 3 - DNA, 714 bp, Artificial Sequence
  Description of Artificial Sequence Synthetic polynucleotide
taatacgactcactatagggctagccccggggatatcgccaccatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcctaatagttctagagcggccgcttccctttagtgagggttaatgcttcgag

SEQ ID NO: 4 - DNA, 783 bp, Artificial Sequence
  Description of Artificial Sequence Synthetic polynucleotide
taatacgactcactatagggctagccccggggatatcgccaccatgccaaaaaagaagagaaaggtggaagaccccggcggtggctctggaggtggtgggtccggcggtggctcttctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcctaatagttctagagcggccgcttccctttagtgagggttaatgcttcgag

SEQ ID NO: 5 - DNA, 24 bp, Artificial Sequence
  Description of Artificial Sequence Synthetic primer
taatacgactcactatagggctag

SEQ ID NO: 6 - DNA, 20 bp, Artificial Sequence
  Description of Artificial Sequence Synthetic primer
ctcgaagcattaaccctcac

SEQ ID NO: 7 - DNA, 19 bp, Artificial Sequence
  Description of Artificial Sequence Synthetic oligonucleotide
tccctatcagtgatagaga

SEQ ID NO: 8 - DNA, 18 bp, Artificial Sequence
  Description of Artificial Sequence Synthetic primer
aacaaggcaaggcttgac

SEQ ID NO: 9 - DNA, 60 bp, Artificial Sequence
  Description of Artificial Sequence Synthetic primer
tattcacggcgcacgagctgcgactctctatcactgatagggaagcatgcctgctattgc

SEQ ID NO: 10 - DNA, 315 bp, Artificial Sequence
  Description of Artificial Sequence Synthetic polynucleotide
agagaccaattgcattcattttatgtttcaggttcagggggaggtgtgggaggttttttaaagcaagtaaaacctctacaaatgtggtaaaatcgataagtccctatcagtgatagagaacggatctacgtcacaacgatccctatcagtgatagagacggttttgtagaagcctaggtccctatcagtgatagagatcaagcgaggtgatttcaactccctatcagtgatagagagcgtacaatcccctaaagtatccctatcagtgatagagaaccacgaagacaggattgtccgatcccatattacgacctt

SEQ ID NO: 11 - DNA, 241 bp, Artificial Sequence
  Description of Artificial Sequence Synthetic polynucleotide
accacgaagacaggattgtccgatcccatattacgacctttccctatcagtgatagagaccattagtcggcacaagtggtccctatcagtgatagagaatgtgttgcgattgcccgcttccctatcagtgatagagagttgccgtacgcgttgaacatccctatcagtgatagagatgcgtatagagcgggtcatttccctatcagtgatagagagtcgacacacaatctcccagctcact

SEQ ID NO: 12 - DNA, 25 bp, Artificial Sequence
  Description of Artificial Sequence Synthetic primer
agagaccaattgcattcattttatg

SEQ ID NO: 13 - DNA, 19 bp, Artificial Sequence
  Description of Artificial Sequence Synthetic primer
agtgagctgggagattgtg

SEQ ID NO: 14 - DNA, 280 bp, Artificial Sequence
  Description of Artificial Sequence Synthetic polynucleotide
caccatgactaaaaaagagttgatagatcgggtagcaaaaaaggccggagcaaagaaaaaagacgtaaaactgatattggatacaatcctggagacaataacagaagcactcgccaagggcgagaaagttcagatcgttggattcggttcatttgaagtgcgaaaagccgcagcaagaaagggagtgaacccacagacccgaaagccaatcactattcctgaaaggaaagtccccaaattcaagcccggtaaggccctcaaggaaaaagttaaatgataa

SEQ ID NO: 15 - DNA, 235 bp, Artificial Sequence
  Description of Artificial Sequence Synthetic polynucleotide
caccatggtatcacaactctacggaatctataggccccagcgcccagacacactcctttctggcgcagacggtgaaagtctcgcacggtacctcgtccaggaggtgcagcttttcggagaagtgcatcccgacctgctgaaccacatcgactacgctgcaatcgggagggagctggagacttcagaaaattatctcttcactgataatggcattttctattaccggtagtgataa

SEQ ID NO: 16 - DNA, 562 bp, Artificial Sequence
  Description of Artificial Sequence Synthetic polynucleotide
caccatgagtcaggacgaatacgagcgattccaggccgccatggaaatcggtgatcacacagggagcatacaagagttgatcaatcttaccgaaaatttggattgttacgacgtgtatcctgacatccatgaccatgatgatcttggaaggtattatatagaagagctggatgcaatgcaagttcccgaacatctgaggaattacatagactatgaagcatatggccgggacatagccttggaagagtctgggcagttcactgatttgggttatgtgagggacacaggcgattcctttcacgagtactatgatggagaacgcggtagtattccagaggaatacagagtgatgactttccaagatgatattcctgaagaagagatatccgaatgggcaatggatctcgcttatgacatggatgaatttttcagacaaaacgaccctcaatacgccgcagaacacccagaggaacatgccgctaaggaagaaatatatgaaaacctgatggcagggcggattagtgctttggatgagaagttggccgctcttgggtagtgataa

SEQ ID NO: 17 - DNA, 354 bp, Artificial Sequence
  Description of Artificial Sequence Synthetic polynucleotide
atggcaatgagcaatatgacatataacaatgttttcgaccacgcttatgagatgctcaaggaaaacatcagatatgacgacatacgcgacacagatgacctgcacgacgcaattcatatggcagccgataatgccgtccctcattactatgcagacattttctcagttatggcatccgagggtattgatctggagtttgaggactcaggccttatgccagacactaaggatgtcatacggatcttgcaagcccggatctacgagcagcttactatagacctctgggaggacgcagaggacctcctgaacgagtatctggaggaagtcgaagagtacgaagaagacgaggaatag

SEQ ID NO: 18 - DNA, 498 bp, Artificial Sequence
  Description of Artificial Sequence Synthetic polynucleotide
atggatgacatgcaagtttacattgcaaacttggggaagtacaatgagggggaactggtgggtgcatggttcaccttcccaatcgacttcgaggaagtaaaagagaaaatcggacttaatgacgagtacgaagagtatgcaatccacgattacgagctgccatttaccgtcgacgaatacactagcatcggagaacttaataggctttgggaaatggtttccgagttgcccgaagaactccagtcagaactttccgcacttcttacccacttcagcagtatagaagaactgtcagaacaccaagaagacatcataatacatagtgattgcgatgatatgtacgatgtggccaggtactacatcgaagagactggggctttgggtgaggtccccgctagtctccaaaattatatagattaccaagcctacggccgcgatcttgacctgtcagggacttttatttctactaaccacggaatcttcgagatcgtttactag

SEQ ID NO: 19 - DNA, 408 bp, Artificial Sequence
  Description of Artificial Sequence Synthetic polynucleotide
atgactaaaaccaactccgaaatattggagcaactgaagcaggctagcgacggacttctgtttatgtcagagtccgaatacccctttgaagtattcctgtgggaggggtccgctccacctgtcactcacgagatcgtcttgcagcagaccggacacggtcaggacgcaccattcaaagttgtggacatcgactccttttttagccgagcaacaacaccccaagattggtacgaagacgaagagaatgctgttgtggcaaagtttcagaaattgctcgaagtcatcaagagtaaccttaaaaatccccaggtgtatcgattgggagaagtggagctggatgtgtacgtcataggggaaacacccgccggtaacctggccgggatctctactaaggtcgttgaaacatag

SEQ ID NO: 20 - DNA, 324 bp, Artificial Sequence
  Description of Artificial Sequence Synthetic polynucleotide
atgacaaccgagattaagaaactggacccagatacagcaatcgacatagcttacgatattttccttgagatggccggtgaaaacctcgatcccgctgacatacttctgttcaacctccagtttgaagagagggggggcgtagaatttgtcgaaaccgcagatgactgggaggaggagattggagtcttgatcgaccctgaagagtacgccgaggtgtgggttggcctggtcaacgagcaagacgagatggacgacgtctttgctaaatttcttatctcacaccgagaggaggaccgagagttccatgtcatttggaagaaatag

SEQ ID NO: 21 - DNA, 357 bp, Artificial Sequence
  Description of Artificial Sequence Synthetic polynucleotide
atgaacgagcacaaccttttgatcttctgcctgaaggacaatgtctctataagtgagtacacagagatgattgattgggcttacaagaatatccagtccgaaactgttgtagaaataaccgagaaccagattatagaatatcagaaccgggggttgtggagactcgtctctgaaattactgacaactggctgttcggtcccagtgaaggggattggcttatagacaaggaatctatacttgctgtcaaagaaaagttgcagaactccgacttctcaaccgagcctcttgtcaaaaacataatccacgtgttggaatacgctatcaagaatgagaagaccgttatctttcacttttag

SEQ ID NO: 22 - DNA, 552 bp, Artificial Sequence
  Description of Artificial Sequence Synthetic polynucleotide
atgcagcaatgggttgattgcgagtttacaggacgggattttagggatgaagacttgtctaggctgcatactgagagggccatgttcagcgagtgcgatttttccggcgtgaatctggctgaaagtcagcatcggggaagtgcatttcgcaattgcacctttgagcgaacaaccctttggcattcaacttttgctcaatgtagcatgctgggtagtgtgttcgtagcatgtcgactcagacccctcactctcgacgatgtcgacttcaccttggccgtgcttggggggaatgacctccgggggttgaacttgactggttgccgattgcgggaaacatctttggttgacactgatctccgaaaatgtgttctgcgcggggctgacctctccggcgctcggactacaggtgcaaggttggacgacgctgacttgaggggtgctaccgtggacccagtgctttggcgaactgcatcccttgtgggagcacgggtcgatgtcgaccaagccgtagcttttgcagcagcccacggactgtgtttggccggaggctag

SEQ ID NO: 23 - DNA, 279 bp, Artificial Sequence
  Description of Artificial Sequence Synthetic polynucleotide
atgattatagactcccagagcgttgtccaatacacttttaagatagacatcctcgagaagctctataaatttttgcccaacctttaccattcaatcgtcaatgagctggtcgaagaacttcatctggagaacaacgacttcctgatagggacatataaagaccttagtaaagcaggttatttttacgtcataccagcacccggcaagaatatcgacgatgtgttgaagacaataatgatttacgtccacgattacgaaattgaggattattttgagtag

SEQ ID NO: 24 - DNA, 417 bp, Artificial Sequence
  Description of Artificial Sequence Synthetic polynucleotide
atggacataaatactgagactgagataaagcagaaacattcactcacaccctttcccgttttcctcataagtccagctttccgggggaggtattttcactcctacttccgctccagtgcaatgaacgcttattacatccaagaccgactggaagcccaaagctgggcccggcactatcagcaactcgctcgggaagagaaagaagcagagcttgccgatgatatggaaaaaggtttgccacaacacttgttcgagtccctgtgcatagaccatttgcaacggcatggtgcatcaaaaaaatctattacccgcgcctttgacgatgatgtagagtttcaagaaaggatggcagagcacattaggtacatggtagagaccattgctcaccatcaagtggatatagactccgaggtgtag

SEQ ID NO: 25 - DNA, 504 bp, Artificial Sequence
  Description of Artificial Sequence Synthetic polynucleotide
atgatagatgacatggcagtatacattgcaaatttgggtaagtataacgaaggctatttggtgggtgcctggtttaccttccccattgacgaagaagatgttaaagaaaagataggactcaacgaacagtacgaagagtatgcaatccatgatactgataacttccccattgcaataggtgagtatgttagcatagaggaactcaacgaaatgtacgaaatgattgaggaactgcccgactatattgtcgaatgtctcgatgagtttatttcacactacgggaccttggaggaagtcgtcgaacacaaagacgatatttactactatccagattgtgagacaatgactgacgtagcctgttactacatagatgagttgcaagcattgggcgacataccacctagtctccaaaactacatcgactatgaagcatatggaagagatttggacatgggcgggtgtttcatcgagacaagccgagggatgtgcgaaattccatattag

SEQ ID NO: 26 - DNA, 294 bp, Artificial Sequence
  Description of Artificial Sequence Synthetic polynucleotide
atgaagtcagacttgcaagagattctcaatgatgcattggacgaattgaaggagcgaatgaaggattatcccgatgaggacgctgatgacgttgtaagcgagatagcagacagtagcgttccagtctactactctgacctcttgaagctggcttccggctgcaatgaccttgctaccgctgaaccagaatgcggaccagcattcgacgggaagccaacccctgtgaatattattgcagctaatgtttacgaagcagtcgatcaacatctccgaaattacttgtcagccatttag

SEQ ID NO: 27 - DNA, 336 bp, Artificial Sequence
  Description of Artificial Sequence Synthetic polynucleotide
atgagtcaaagtttgtacgagattattaaacttgctagggaagagcttcggggacgagcaaaggataacaaagatgagaccgagccccacgactctatccacgagattgctgattcatctgtgccagtctatacaggcgatctcttgcagttggcagcagacaacttggagctggccacagctaaaccagagcttggacccgcctttgatggcagtcccacaccagtcaacatcgtggctgctaatgtatttgaagccattgaagctgggctgtgggaagaatggaaggaaatcgagtcagaacgcgaagatgcagagttggaagaaactggctag

SEQ ID NO: 28 - DNA, 606 bp, Artificial Sequence
  Description of Artificial Sequence Synthetic polynucleotide
atgactagtatagctcgccccgatatagtggatcgcgttttggccgccgcagcagacagggccagggaactcgtcgccgaggaaaggcgcctcattgcagaagacgctattgatgtagaggtggtcgtacgcactgatagatccagtggcgatgttgtaacctctcgcaagggacgctcctcctttgccaccgaagagccagttttgttggatgagtcaccaagcgcaaaacatagtgcagtgcgacctaagggtgatgatatgtctgacgagaagcgaacaaccctctacgggctggagcgcggagtacgagatgaagttagagagcgcagcaaggaacttctggaggacgtgtgcccagaagacaccctgaccgagatcgctgatgggtgggtaccaatctacacttacaacatactccaggtcgctgcagacaacatggacatggcaaccctggagcctgaactgggacccgcattcgatggcacacctacccctatcaacataattgccgccaacatatataaggcactcaatgccgcagccttcaaagaatgggctaaagttcatcccaaatggcgcaaaaagctggccgggaacgattag

SEQ ID NO: 29 - DNA, 213 bp, Artificial Sequence
  Description of Artificial Sequence Synthetic polynucleotide
atgtggatgatgggggagctgcctattgcccctgttgaccgattgataagaaaggccggagcagagagggtgtctgaacaggccgcaaaggtcctcgccgaatatctcgaggagtatgcaatagagatcgccaaaaaagcagtagagttcgctagacatgcaggtcgcaaaaccgttaaagtagaagacatcaaattggctattaagtcctga

SEQ ID NO: 30 - DNA, 342 bp, Artificial Sequence
  Description of Artificial Sequence Synthetic polynucleotide
atggcccccaaaatgaaggccgctatgaaagctaaagcaatgaaggcacggtcagtagccatgagtaagggcgctctttgtcaagcaatagccgatgctacagagaataagaagagtgccattgttaaatttatggatgcccttgccgaggtagttactgctgaggtcaaaaagaccgggaaaatgacaatacctggggtcacaatgattaaaaccagaaaaaaacctgcaacaaaagcagggaagcgggaaatgtttggaaaggtggtgctggtaaaggcccaacctgccaagacagttgtgaaagcctttcccgttaaagccttgaagacagacttttga

SEQ ID NO: 31 - DNA, 273 bp, Artificial Sequence
  Description of Artificial Sequence Synthetic polynucleotide
atgaataaaacacagcttatagatgtaatcgccgaaaaagcagagctttcaaagacacaggctaaagcagctctggaaagcactcttgctgctattaccgagagcctcaaggaaggagatgcagttcaactggtaggatttgggacctttaaggtcaatcatagagccgaaagaaccggacgcaacccacagactggtaaagagataaaaattgctgccgcaaacgtacctgcattcgtatccgggaaagcccttaaggatgctgtcaagtga

SEQ ID NO: 32 - DNA, 294 bp, Artificial Sequence
  Description of Artificial Sequence Synthetic polynucleotide
atggacgaacccaagaaatcagtggccttcaaaaagaccaagaaggaaatcaagaaggtagccacgccaaaaaaggcatccaagcccaagaaggctgcctccaaagccccaaccaagaaacccaaagccaccccggtcaaaaaggccaagaaaaagctggctgccacgcccaagaaagctaaaaaacccaagactgtcaaagccaagccggtcaaggcatccaagcccaaaaaggccaaaccagtgaaacccaaagcaaagtccagtgccaagagggccggcaaaaagaaataa

SEQ ID NO: 33 - protein, 27 aa, Simian virus 40
PSSDDEATADSQHSTPPKKKRKVEDPK

SEQ ID NO: 34 - protein, 18 aa, Artificial Sequence
  Description of Artificial Sequence Synthetic peptide
PAAKRVKLDPAAKRVKLD

SEQ ID NO: 35 - protein, 16 aa, Artificial Sequence
  Description of Artificial Sequence Synthetic peptide
PKKKRKVPAAKRVKLD

SEQ ID NO: 36 - protein, 117 aa, Artificial Sequence
  Description of Artificial Sequence Synthetic polypeptide
MAMSNMTYNNVFDHAYEMLKENIRYDDIRDTDDLHDAIHMAADNAVPHYYADIFSVMASEGIDLEFEDSGLMPDTKDVIRILQARIYEQLTIDLWEDAEDLLNEYLEEVEEYEEDEE

SEQ ID NO: 37 - DNA, 364 bp, Artificial Sequence
  Description of Artificial Sequence Synthetic polynucleotide
caccatggcaatgagcaatatgacatataacaatgttttcgaccacgcttatgagatgctcaaggaaaacatcagatatgacgacatacgcgacacagatgacctgcacgacgcaattcatatggcagccgataatgccgtccctcattactatgcagacattttctcagttatggcatccgagggtattgatctggagtttgaggactcaggccttatgccagacactaaggatgtcatacggatcttgcaagcccggatctacgagcagcttactatagacctctgggaggacgcagaggacctcctgaacgagtatctggaggaagtcgaagagtacgaagaagacgaggaatagtgataa

SEQ ID NO: 38 - protein, 111 aa, Artificial Sequence
  Description of Artificial Sequence Synthetic polypeptide
MSQSLYEIIKLAREELRGRAKDNKDETEPHDSIHEIADSSVPVYTGDLLQLAADNLELATAKPELGPAFDGSPTPVNIVAANVFEAIEAGLWEEWKEIESEREDAELEETG

SEQ ID NO: 39 - DNA, 346 bp, Artificial Sequence
  Description of Artificial Sequence Synthetic polynucleotide
caccatgagtcaaagtttgtacgagattattaaacttgctagggaagagcttcggggacgagcaaaggataacaaagatgagaccgagccccacgactctatccacgagattgctgattcatctgtgccagtctatacaggcgatctcttgcagttggcagcagacaacttggagctggccacagctaaaccagagcttggacccgcctttgatggcagtcccacaccagtcaacatcgtggctgctaatgtatttgaagccattgaagctgggctgtgggaagaatggaaggaaatcgagtcagaacgcgaagatgcagagttggaagaaactggctagtgataa

SEQ ID NO: 40 - protein, 97 aa, Artificial Sequence
  Description of Artificial Sequence Synthetic polypeptide
MKSDLQEILNDALDELKERMKDYPDEDADDVVSEIADSSVPVYYSDLLKLASGCNDLATAEPECGPAFDGKPTPVNIIAANVYEAVDQHLRNYLSAI

SEQ ID NO: 41 - DNA, 304 bp, Artificial Sequence
  Description of Artificial Sequence Synthetic polynucleotide
caccatgaagtcagacttgcaagagattctcaatgatgcattggacgaattgaaggagcgaatgaaggattatcccgatgaggacgctgatgacgttgtaagcgagatagcagacagtagcgttccagtctactactctgacctcttgaagctggcttccggctgcaatgaccttgctaccgctgaaccagaatgcggaccagcattcgacgggaagccaacccctgtgaatattattgcagctaatgtttacgaagcagtcgatcaacatctccgaaattacttgtcagccatttagtgataa

SEQ ID NO: 42 - protein, 165 aa, Artificial Sequence
  Description of Artificial Sequence Synthetic polypeptide
MDDMQVYIANLGKYNEGELVGAWFTFPIDFEEVKEKIGLNDEYEEYAIHDYELPFTVDEYTSIGELNRLWEMVSELPEELQSELSALLTHFSSIEELSEHQEDIIIHSDCDDMYDVARYYIEETGALGEVPASLQNYIDYQAYGRDLDLSGTFISTNHGIFEIVY

SEQ ID NO: 43 - DNA, 508 bp, Artificial Sequence
  Description of Artificial Sequence Synthetic polynucleotide
caccatggatgacatgcaagtttacattgcaaacttggggaagtacaatgagggggaactggtgggtgcatggttcaccttcccaatcgacttcgaggaagtaaaagagaaaatcggacttaatgacgagtacgaagagtatgcaatccacgattacgagctgccatttaccgtcgacgaatacactagcatcggagaacttaataggctttgggaaatggtttccgagttgcccgaagaactccagtcagaactttccgcacttcttacccacttcagcagtatagaagaactgtcagaacaccaagaagacatcataatacatagtgattgcgatgatatgtacgatgtggccaggtactacatcgaagagactggggctttgggtgaggtccccgctagtctccaaaattatatagattaccaagcctacggccgcgatcttgacctgtcagggacttttatttctactaaccacggaatcttcgagatcgtttactagtgataa

SEQ ID NO: 44 - protein, 167 aa, Artificial Sequence
  Description of Artificial Sequence Synthetic polypeptide
MIDDMAVYIANLGKYNEGYLVGAWFTFPIDEEDVKEKIGLNEQYEEYAIHDTDNFPIAIGEYVSIEELNEMYEMIEELPDYIVECLDEFISHYGTLEEVVEHKDDIYYYPDCETMTDVACYYIDELQALGDIPPSLQNYIDYEAYGRDLDMGGCFIETSRGMCEIPY

SEQ ID NO: 45 - DNA, 514 bp, Artificial Sequence
  Description of Artificial Sequence Synthetic polynucleotide
caccatgatagatgacatggcagtatacattgcaaatttgggtaagtataacgaaggctatttggtgggtgcctggtttaccttccccattgacgaagaagatgttaaagaaaagataggactcaacgaacagtacgaagagtatgcaatccatgatactgataacttccccattgcaataggtgagtatgttagcatagaggaactcaacgaaatgtacgaaatgattgaggaactgcccgactatattgtcgaatgtctcgatgagtttatttcacactacgggaccttggaggaagtcgtcgaacacaaagacgatatttactactatccagattgtgagacaatgactgacgtagcctgttactacatagatgagttgcaagcattgggcgacataccacctagtctccaaaactacatcgactatgaagcatatggaagagatttggacatgggcgggtgtttcatcgagacaagccgagggatgtgcgaaattccatattagtgataa

SEQ ID NO: 46 - protein, 74 aa, Artificial Sequence
  Description of Artificial Sequence Synthetic polypeptide
MVSQLYGIYRPQRPDTLLSGADGESLARYLVQEVQLFGEVHPDLLNHIDYAAIGRELETSENYLFTDNGIFYYR

SEQ ID NO: 47 - DNA, 235 bp, Artificial Sequence; identical to SEQ ID NO: 15
  Description of Artificial Sequence Synthetic polynucleotide

SEQ ID NO: 48 - protein, 183 aa, Artificial Sequence
  Description of Artificial Sequence Synthetic polypeptide
MSQDEYERFQAAMEIGDHTGSIQELINLTENLDCYDVYPDIHDHDDLGRYYIEELDAMQVPEHLRNYIDYEAYGRDIALEESGQFTDLGYVRDTGDSFHEYYDGERGSIPEEYRVMTFQDDIPEEEISEWAMDLAYDMDEFFRQNDPQYAAEHPEEHAAKEEIYENLMAGRISALDEKLAALG

SEQ ID NO: 49 - DNA, 562 bp, Artificial Sequence; identical to SEQ ID NO: 16
  Description of Artificial Sequence Synthetic polynucleotide

SEQ ID NO: 50 - DNA, 26 bp, Artificial Sequence
  Description of Artificial Sequence Synthetic primer
cgaaattaatacgactcactataggg

SEQ ID NO: 51 - DNA, 136 bp, Artificial Sequence
  Description of Artificial Sequence Synthetic primer
tttttttttttttttttttttttttttttttttttttttttttttttttttttttttttttttttttttttttttttttttttttttttttttttttttttttttttttttttttttttttcgaggctgatcagcg

SEQ ID NO: 52 - DNA, 48 bp, Artificial Sequence
  Description of Artificial Sequence Synthetic primer
aatggttaatacgactcactatagggcaccttgttctttttgcagaag

SEQ ID NO: 53 - DNA, 141 bp, Artificial Sequence
  Description of Artificial Sequence Synthetic primer
ttttttttttttttttttttttttttttttttttttttttttttttttttttttttttttttttttttttttttttttttttttttttttttttttttttttttttttttttttttttttcttcctactcaggctttattc

SEQ ID NO: 54 - DNA, 834 bp, Artificial Sequence
  Description of Artificial Sequence Synthetic polynucleotide
caccttgttctttttgcagaagctcagaataaacgctcaactttggcctagccgctataattgtttctatgccgagtaatgagaacaaccacaccatagcgatttaacgcagcgcctcggaataccgttttagcaggcgcttgctaagaccattgcgaattccaggtatcgtgtatgtagcgtagggccatacgcaagttaaactgctaggaaaccgcgtttctacgaccggtgcataatttaatttcgctgacgtgatgacattcctgctaatgcctcacctgtcggatccctctcgtgatagggtagttggacatgtccttgtaagatataacaagagcctgcctgtttaatgatctcacggcgaaagtcggggagacagcagcggctgcagacattatatcgcaataatactaaggtgagataactccgtaattgactacgcatttctctagactttacttgaccagatacagtgactttgacacgtttatggattacagcaatcacatccaagactgcctatggaggaagcaactcttgagtgttaatatgttgactcctgtattagggatgcaggtagtagatgagtgcagggacaccgaggttaagtacattaccctctcataggaggtgttctagatcaccataccaccatattattcgagcatgacattatctgcgctgtccccatcctagtagtcattattcctattacgcttttgagtgactggtgacggagctgccttctgcggggcttgccttctggccatgcccttcttctctcccttgcacctgtacctcttggtctttgaataaagcctgagtaggaag

SEQ ID NO: 55 - DNA, 78 bp, Artificial Sequence
  Description of Artificial Sequence Synthetic primer
cttgttctttttgcagaagctcagaataaacgctcaactttggccaccatggaagatgccaaaaacattaagaagggc

SEQ ID NO: 56 - DNA, 157 bp, Artificial Sequence
  Description of Artificial Sequence Synthetic primer
agaatgtgaagaaactttctttttattaggagcagatacgaatggctacattttgggggacaacattttgtaaagtgtaagttggtattatgtagcttagagactccattcgggtgttcttgaggctggtctatcattacacggcgatcttgccgcc

SEQ ID NO: 57 - DNA, 45 bp, Artificial Sequence
  Description of Artificial Sequence Synthetic primer
gaatttaatacgactcactataaggcttgttctttttgcagaagc

SEQ ID NO: 58 - DNA, 149 bp, Artificial Sequence
  Description of Artificial Sequence Synthetic primer
ttttttttttttttttttttttttttttttttttttttttttttttttttttttttttttttttttttttttttttttttttttttttttttttttttttttttttttttttttttttttagaatgtgaagaaactttctttttattag

SEQ ID NO: 59 - RNA, 1980 nt, Artificial Sequence
  Description of Artificial Sequence Synthetic polynucleotide
aggcuuguucuuuuugcagaagccuuguucuuuuugcagaagcucagaauaaacgcucaacuuuggccaccauggaagaugccaaaaacauuaagaagggcccagcgccauucuacccacucgaagacgggaccgccggcgagcagcugcacaaagccaugaagcgcuacgcccuggugcccggcaccaucgccuuuaccgacgcacauaucgagguggacauuaccuacgccgaguacuucgagaugagcguucggcuggcagaagcuaugaagcgcuaugggcugaauacaaaccaucggaucguggugugcagcgagaauagcuugcaguucuucaugcccguguugggugcccuguucaucgguguggcuguggccccagcuaacgacaucuacaacgagcgcgagcugcugaacagcaugggcaucagccagcccaccgucguauucgugagcaagaaagggcugcaaaagauccucaacgugcaaaagaagcuaccgaucauacaaaagaucaucaucauggauagcaagaccgacuaccagggcuuccaaagcauguacaccuucgugacuucccauuugccacccggcuucaacgaguacgacuucgugcccgagagcuucgaccgggacaaaaccaucgcccugaucaugaacaguaguggcaguaccggauugcccaagggcguagcccuaccgcaccgcaccgcuuguguccgauucagucaugcccgcgaccccaucuucggcaaccagaucauccccgacaccgcuauccucagcguggugccauuucaccacggcuucggcauguucaccacgcugggcuacuugaucugcggcuuucgggucgugcucauguaccgcuucgaggaggagcuauucuugcgcagcuugcaagacuauaagauucaaucugcccugcuggugcccacacuauuuagcuucuucgcuaagagcacucucaucgacaaguacgaccuaagcaacuugcacgagaucgccagcggcggggcgccgcucagcaaggagguaggugaggccguggccaaacgcuuccaccuaccaggcauccgccagggcuacggccugacagaaacaaccagcgccauucugaucacccccgaaggggacgacaagccuggcgcaguaggcaagguggugcccuucuucgaggcuaaggugguggacuuggacaccgguaagacacugggugugaaccagcgcggcgagcugugcguccguggccccaugaucaugagcggcuacguuaacaaccccgaggcuacaaacgcucucaucgacaaggacggcuggcugcacagcggcgacaucgccuacugggacgaggacgagcacuucuucaucguggaccggcugaagagccugaucaaauacaagggcuaccagguagccccagccgaacuggagagcauccugcugcaacaccccaacaucuucgacgccggggucgccggccugcccgacgacgaugccggcgagcugcccgccgcagucgucgugcuggaacacgguaaaaccaugaccgagaaggagaucguggacuauguggccagccagguuacaaccgccaagaagcugcgcggugguguuguguucguggacgaggugccuaaaggacugaccggcaaguuggacgcccgcaagauccgcgagauucucauuaaggccaagaagggcggcaagaucgccguguaaugauagaccagccucaagaacacccgaauggagucucuaagcuacauaauaccaacuuacacuuuacaaaauguugucccccaaaauguagccauucguaucugcuccuaauaaaaagaaaguuucuucacauucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 60 - DNA, 670 bp, Artificial Sequence
  Description of Artificial Sequence Synthetic polynucleotide
cttgttctttttgcagaagctcagaataaacgctcaactttggccaccatgagccagacccaacccccggccccagttgggccgggcgacccagatgtttacttaaaaggcgtgccgtccgccggcatgcaccccagaggtgttcacgcacctcgaggacacccgcgcatgatctccggacccccgcaacggggtgataacgatcaagcggcggggcaatgtggagattcgggtctactacgagtcggtgcggacactacgatctcgaagccatctgaagccgtccgaccgccaacaatccccaggacaccgcgtgttccccgggagccccgggttccgcgaccaccccgagaacctagggaacccagagtaccgcgagctcccagagaccccagggtaccgcgtgaccccagggatccacgacaaccgcgttccccaagggagccccggtctccccgtgaaccccggtctcccagggagccccggaccccacgcaccccccgcgaaccacgtacggctcgcggttctgtataatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 61 - RNA, 813 nt, Artificial Sequence
  Description of Artificial Sequence Synthetic polynucleotide
aggcuuguucuuuuugcagaagccuuguucuuuuugcagaagcucagaauaaacgcucaacuuuggccaccaugagccagacccaacccccggccccaguugggccgggcgacccagauguuuacuuaaaaggcgugccguccgccggcaugcaccccagagguguucacgcaccucgaggacacccgcgcaugaucuccggacccccgcaacggggugauaacgaucaagcggcggggcaauguggagauucgggucuacuacgagucggugcggacacuacgaucucgaagccaucugaagccguccgaccgccaacaauccccaggacaccgcguguuccccgggagccccggguuccgcgaccaccccgagaaccuagggaacccagaguaccgcgagcucccagagaccccaggguaccgcgugaccccagggauccacgacaaccgcguuccccaagggagccccggucuccccgugaaccccggucucccagggagccccggaccccacgcaccccccgcgaaccacguacggcucgcgguucuguauaaugauagaccagccucaagaacacccgaauggagucucuaagcuacauaauaccaacuuacacuuuacaaaauguugucccccaaaauguagccauucguaucugcuccuaauaaaaagaaaguuucuucacauucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - DNA, 463 bp, Artificial Sequence
  Description of Artificial Sequence Synthetic polynucleotide
taacttgttctttttgcagaagctcagaataaacgctcaactttggccaccatgctagtcaacctccttaaggacgagcgtcgggcgaacatccgcgccaaacggcacttgacactgcaggtccagatcaatcggcaacaagccgtcaaggaaacctctgtagcattgaggacggagttgcacaacctgaggctaatcacattgaataaagaaccgcaatttgttaaagccaaaaaccgatcctttatcgacagggagcaggagtctaaattgtgtgaaaacgcaaagtaccagagcgagcttcccaagattaaagaagaggaataatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 63 - DNA, 463 bp, Artificial Sequence
  Description of Artificial Sequence Synthetic polynucleotide
taacttgttctttttgcagaagctcagaataaacgctcaactttggccaccatgaacaagaactctatccggcaatctcttaaaaacgttcggttggacgaagtcgcaaacgcacacttgcaacagagggaagtcaaaccgatcgacatcaagcgcctgagcaaagccgagaacaaatttcaatacttgagggtcgaaaagcagacactattggccgagcccaaagagtgtacggagcgtaataaggagtttcaggtagaactgaggacacgagagcagcgggcccacttgattaccctacttctcgaaatcaaagcgaattcctaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 64 - RNA, 603 nt, Artificial Sequence
  Description of Artificial Sequence Synthetic polynucleotide
aggcuuguucuuuuugcagaagccuuguucuuuuugcagaagcucagaauaaacgcucaacuuuggccaccaugcuagucaaccuccuuaaggacgagcgucgggcgaacauccgcgccaaacggcacuugacacugcagguccagaucaaucggcaacaagccgucaaggaaaccucuguagcauugaggacggaguugcacaaccugaggcuaaucacauugaauaaagaaccgcaauuuguuaaagccaaaaaccgauccuuuaucgacagggagcaggagucuaaauugugugaaaacgcaaaguaccagagcgagcuucccaagauuaaagaagaggaauaaugauagaccagccucaagaacacccgaauggagucucuaagcuacauaauaccaacuuacacuuuacaaaauguugucccccaaaauguagccauucguaucugcuccuaauaaaaagaaaguuucuucacauucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - RNA, 603 nt, Artificial Sequence
  Description of Artificial Sequence Synthetic polynucleotide
aggcuuguucuuuuugcagaagccuuguucuuuuugcagaagcucagaauaaacgcucaacuuuggccaccaugaacaagaacucuauccggcaaucucuuaaaaacguucgguuggacgaagucgcaaacgcacacuugcaacagagggaagucaaaccgaucgacaucaagcgccugagcaaagccgagaacaaauuucaauacuugagggucgaaaagcagacacuauuggccgagcccaaagaguguacggagcguaauaaggaguuucagguagaacugaggacacgagagcagcgggcccacuugauuacccuacuucucgaaaucaaagcgaauuccuaaugauagaccagccucaagaacacccgaauggagucucuaagcuacauaauaccaacuuacacuuuacaaaauguugucccccaaaauguagccauucguaucugcuccuaauaaaaagaaaguuucuucacauucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 66 - protein, 7 aa, Simian virus 40
PKKKRKV

SEQ ID NO: 67 - protein, 9 aa, Unknown
  Description of Unknown C-myc NLS sequence
PAAKRVKLD

SEQ ID NO: 68 - protein, 9 aa, Unknown
  Description of Unknown Tus NLS peptide
KLKIKRPVK

SEQ ID NO: 69 - DNA, 25 bp, Artificial Sequence
  Description of Artificial Sequence Synthetic oligonucleotide
ttttttttttttttttttttttttt

(19) United States
(12) Patent Application Publication
(10) Pub. No.: US 2020/0224194 A1
(43) Pub. Date: Jul. 16, 2020

(54) EXPRESSION SYSTEMS THAT FACILITATE NUCLEIC ACID DELIVERY AND METHODS OF USE

(71) Applicant: Helix Nanotechnologies, Inc., Walnut, CA (US)

(72) Inventors: Nikolai Eroshenko, Boston, MA (US); Nikhil Dhar, Boston, MA (US); Taylor Gill, Cambridge, MA (US); Marianna Keaveney, Walpole, MA (US); Hannu Rajaniemi, San Francisco, CA (US)

(21) Appl. No.: 16/649,105

(22) PCT Filed: Sep. 20, 2018
(86) PCT No.: PCT/US2018/052077

§ 371 Date: Mar. 19, 2020

Related U.S. Application Data
(60) Provisional application No. 62/561,131, filed on Sep. 20, 2017.
(60) Provisional application No. 62/720,105, filed on Aug. 20, 2018.

(51) Int. Cl. C12N 15/11; C12N 15/79; A61K 48/00; C07K 14/705

(52) U.S. Cl. CPC C12N 15/11; C07K 14/705; A61K 48/005; C12N 15/79

(57) ABSTRACT

Nucleic acid expression systems are provided herein that include a first synthetic oligonucleotide comprising a payload sequence and a second synthetic oligonucleotide comprising a sequence that encodes a helper polypeptide. Compositions (e.g., pharmaceutical compositions) comprising the nucleic acid expression systems as well as methods of using the same are also provided herein.

### CROSS-REFERENCE TO RELATED APPLICATIONS

This application is a national stage filing under U.S.C. § 371 of PCT International Application No. PCT/US2018/052077 filed Sep. 20, 2018, which claims priority to and benefit of U.S. Provisional Patent Application No. 62/561,131, filed Sep. 20, 2017, and U.S. Provisional Patent Application No. 62/720,105, filed Aug. 20, 2018, the contents of each of which are hereby incorporated by reference herein in their entirety.

### SEQUENCE LISTING

The instant application contains a Sequence Listing which has been submitted electronically in ASCII format and is hereby incorporated by reference in its entirety. Said ASCII copy, created on Dec. 28, 2018, is named 2012611-0018_SL.txt and is 48,475 bytes in size.

### BACKGROUND

In recent years, gene therapy, or the idea to genetically modify the cells of a patient to treat or improve a particular condition or disease, has seen great progress. Modern gene therapy approaches are generally based on one of three approaches: engineered viruses, non-viral DNA vectors, or RNAs. However, each of these approaches currently have significant technological limitations.

### SUMMARY

The present disclosure provides technologies for enhancing efficacy of gene therapies, in particular non-viral gene therapies. Among other things, the present disclosure recognizes that gene therapies must overcome significant obstacles including, for example, transport and targeting of an oligonucleotide, uptake by target cells, perseverance, and translocation to the nucleus. The present disclosure provides the insight that efficacy of a gene therapy may be enhanced (e.g., expression, nuclear import, persistence or uptake of a payload oligonucleotide may be increased) by co-expression of one or more helper proteins.

The present disclosure provides the insight that use of helper proteins that employ viral mechanisms may enhance expression, nuclear import, persistence or uptake of a payload oligonucleotide. In some embodiments, a viral mechanism includes one or more of: increasing nuclear localization, suppressing innate immunity, reducing degradation of payload oligonucleotides and increasing uptake of a payload oligonucleotide. In some embodiments, a helper protein in the context of the present disclosure mimics a viral mechanism to enhance expression, nuclear import, persistence or uptake of a non-viral oligonucleotide. In some embodiments, a viral mechanism to enhance expression of a non-viral nucleotide includes one or more of increasing nuclear localization, increasing persistence of the oligonucleotide, and suppressing innate immunity.

The present disclosure encompasses the insight that efficacy of a gene therapy may be enhanced (e.g., expression of a payload oligonucleotide may be increased) by “jumpstarting” the system by transient expression of one or more helper proteins encoded by RNA (e.g., mRNA) oligonucleotide(s). In some embodiments, one or more helper proteins enhance one or more of: nuclear localization (e.g., through a nuclear localization signal (NLS) polypeptide), persistence (e.g., as a DNA mimic protein or by suppressing innate immunity), and uptake of the payload (e.g., by a synthetic cell surface receptor polypeptide).

In some embodiments, a nucleic acid expression system includes an oligonucleotide comprising a payload sequence and at least one oligonucleotide sequence comprising a sequence that encodes a helper polypeptide for enhancing expression of the oligonucleotide comprising a payload sequence in a target cell. In some embodiments, a helper polypeptide is or comprises one or more of the following: a nuclear localization signal (NLS) polypeptide, a DNA mimic polypeptide, a viral modulator of innate immunity, and a synthetic cell surface receptor polypeptide.

In some embodiments, an oligonucleotide comprising a sequence that encodes a helper polypeptide is a synthetic oligonucleotide. In some embodiments, an oligonucleotide comprising a sequence that encodes a helper polypeptide is a DNA (e.g., a cDNA) oligonucleotide. In some embodiments, an oligonucleotide comprising a sequence that encodes a helper polypeptide is an RNA (e.g., an mRNA) oligonucleotide.

In some embodiments, an oligonucleotide comprising a payload sequence is a synthetic oligonucleotide. In some embodiments, an oligonucleotide comprising a payload sequence is a DNA (e.g., a cDNA) oligonucleotide. In some embodiments, an oligonucleotide comprising a payload sequence is an RNA (e.g., an mRNA) oligonucleotide.

In some embodiments, an oligonucleotide comprising a payload sequence comprises homology arms. In some embodiments, a homology arm is or comprises a sequence that is homologous to a target site and/or a region flanking a target site in the genome of a target cell. In some embodiments, a homology arm is 50 bp to 10,000 bp in length.

Without wishing to be bound by theory, it is envisioned that in some embodiments, homology arms can be used as a template for homologous recombination. In some embodiments, a payload sequence from an oligonucleotide that includes one or more homology arms can be inserted into the genome of a target cell via homologous recombination. In some embodiments, a homologous recombination event utilizes the endogenous cell machinery. In some embodiments, a homologous recombination event utilizes an exogenously co-expressed targeted nuclease.

In some embodiments, an oligonucleotide comprising a payload sequence and/or at least one oligonucleotide comprising a sequence that encodes a helper polypeptide are part of a vector.

In some embodiments, a nucleic acid expression system also includes an oligonucleotide encoding a targeted nuclease. In some embodiments, an oligonucleotide encoding a targeted nuclease is a DNA (e.g., a cDNA) oligonucleotide. In some embodiments, an oligonucleotide encoding a targeted nuclease is an RNA (e.g., mRNA) oligonucleotide. In some embodiments, a targeted nuclease is a zinc-finger nuclease (ZFN), TAL effector domain nuclease (TALEN), or an engineered CRISPR/Cas9 system.

In some embodiments, provided are vectors comprising one or more of an oligonucleotide comprising a payload sequence, an oligonucleotide comprising a sequence that encodes a helper polypeptide are part of a vector and an oligonucleotide encoding a targeted nuclease. In some embodiments, a vector is a non-viral vector.

In some embodiments, a nucleic acid expression system includes a synthetic DNA oligonucleotide comprising a payload sequence and at least one mRNA oligonucleotide sequence that encodes a helper polypeptide for enhancing expression of the oligonucleotide comprising a payload sequence in a target cell. In some embodiments, a helper polypeptide is or comprises one or more of the following: a nuclear localization signal (NLS) polypeptide, a DNA mimic polypeptide, a viral modulator of innate immunity, and a synthetic cell surface receptor polypeptide.

In some embodiments, a nucleic acid expression system includes a oligonucleotide comprising a payload sequence and a composition that delivers at least one helper polypeptide. In some embodiments, a helper polypeptide is or comprises one or more of the following: a nuclear localization signal (NLS) polypeptide, a DNA mimic polypeptide, a viral modulator of innate immunity, and a synthetic cell surface receptor polypeptide. In some embodiments, a composition that delivers a helper polypeptide is or comprises (i) an oligonucleotide (e.g., DNA (e.g., cDNA) and/or RNA (e.g., mRNA)) that encodes a helper polypeptide and/or (ii) a helper polypeptide.

In some embodiments, a helper polypeptide is or comprises a NLS polypeptide. In some embodiments, a NLS polypeptide is an SV40 NLS or variant thereof. In some embodiments, a NLS polypeptide is from EGL-13, c-Myc, NLP or TUS.

In some embodiments, a NLS polypeptide is operatively connected to a DNA-binding domain (DBD) polypeptide. In some embodiments, a DBD polypeptide is not regulated by a small molecule. In some embodiments, a DBD is or comprises a Cro repressor or a catalytically-inactive meganuclease variant. In some embodiments, a DBD polypeptide is a synthetic DBD. In some embodiments, a DBD is or comprises a zinc finger, a TAL domain, or a catalytically-inactive Cas9. In some embodiments, a DBD polypeptide is a non-specific DBD. In some embodiments, a DBD is or comprises Sso7d, H-NS, HU-1, HU-2, p6 of ϕ29, A104R of ASFV, dsp, TmHU, HPhA, or HCcp3.

In some embodiments, a NLS polypeptide is fused with a DBD. In some embodiments, a NLS polypeptide and DBD are separate polypeptides that can join to form a complex (e.g., dimerize). In some embodiments, a NLS polypeptide and a DBD dimerize through inducible dimerization domains. Exemplary inducible dimerization domains include a rapamycin-inducible FRB/FKBP pair.

In some embodiments, a helper polypeptide is or comprises a DNA mimic polypeptide. In some embodiments, a DNA mimic polypeptide is selected from any one of Ocr, ArdA, NuiA, HI1450, DMP12, MfpA, Am, Gam and/or variants thereof. In some embodiments, a DNA mimicking polypeptide is from bacteriophage. In some embodiments, a helper polypeptide is a fully engineered DNA mimic.

In some embodiments, a helper polypeptide is or comprises a viral modulator of innate immunity. Viral modulator of innate immunity include, for example, vIRF1, ORF52/KicGAS, PLP2-TM, PLP2, US11 and/or variants thereof.

In some embodiments, a helper polypeptide is or comprises a synthetic cell surface receptor polypeptide. Synthetic cell surface receptor polypeptides include, for example, TVA-EGF, H-EGF, H-IGF1 and/or variants thereof.

In some embodiments, (1) an oligonucleotide comprising a payload sequence, and at least one (2)(a) oligonucleotide comprising a sequence that encodes a helper polypeptide or (2)(b) composition that delivers a helper polypeptide are administered sequentially. In some embodiments, (1) an oligonucleotide comprising a payload sequence, and at least one (2)(a) oligonucleotide comprising a sequence that encodes a helper polypeptide or (2)(b) composition that delivers a helper polypeptide are administered concurrently.

In some embodiments, a nucleic acid expression system includes an oligonucleotide comprising a payload sequence and at least one oligonucleotide sequence comprising a sequence that encodes a helper polypeptide for enhancing nuclear import of the oligonucleotide comprising a payload sequence in a target cell. In some embodiments, a helper polypeptide is or comprises a NLS polypeptide.

In some embodiments, a nucleic acid expression system includes a synthetic DNA oligonucleotide comprising a payload sequence and at least one mRNA oligonucleotide sequence that encodes a helper polypeptide for enhancing nuclear import of the oligonucleotide comprising a payload sequence in a target cell. In some embodiments, a helper polypeptide is or comprises a NLS polypeptide.

In some embodiments, a nucleic acid expression system includes an oligonucleotide comprising a payload sequence, an oligonucleotide sequence that encodes a helper polypeptide comprising a nuclear localization signal (NLS) polypeptide, and an oligonucleotide encoding a DNA-binding domain (DBD) polypeptide.

In some embodiments, a NLS polypeptide is an SV40 NLS or variant thereof. In some embodiments, a NLS polypeptide is from EGL-13, c-Myc, NLP or TUS.

In some embodiments, a NLS polypeptide is operatively connected to a DNA-binding domain (DBD) polypeptide. In some embodiments, a DBD polypeptide is not regulated by a small molecule. In some embodiments, a DBD is or comprises a Cro repressor or a catalytically-inactive meganuclease variant. In some embodiments, a DBD polypeptide is a synthetic DBD. In some embodiments, a DBD is or comprises a zinc finger, a TAL domain, or a catalytically-inactive Cas9. In some embodiments, a DBD polypeptide is a non-specific DBD. In some embodiments, a DBD is or comprises Sso7d, H-NS, HU-1, HU-2, p6 of ϕ29, A104R of ASFV, dsp, TmHU, HPhA, or HCcp3.

In some embodiments, a NLS polypeptide is fused with a DBD. In some embodiments, a NLS polypeptide and DBD are separate polypeptides that can join to form a complex (e.g., dimerize). In some embodiments, a NLS polypeptide and a DBD dimerize through inducible dimerization domains. Exemplary inducible dimerization domains include a rapamycin-inducible FRB/FKBP pair.

In some embodiments, an oligonucleotide comprising a payload sequence and at least one oligonucleotide comprising a sequence that encodes a helper polypeptide or composition that delivers a helper polypeptide are administered sequentially. In some embodiments, an oligonucleotide comprising a payload sequence and at least one oligonucleotide comprising a sequence that encodes a helper polypeptide or composition that delivers a helper polypeptide are administered concurrently.

In some embodiments, a nucleic acid expression system includes an oligonucleotide comprising a payload sequence and at least one oligonucleotide sequence comprising a sequence that encodes a helper polypeptide for enhancing persistence or uptake of the oligonucleotide comprising a payload sequence in a target cell. In some embodiments, a helper polypeptide is or comprises one or more of the following: a DNA mimic polypeptide, a viral modulator of innate immunity, and a synthetic cell surface receptor polypeptide.

In some embodiments, an oligonucleotide comprising a sequence that encodes a helper polypeptide is a synthetic oligonucleotide. In some embodiments, an oligonucleotide comprising a sequence that encodes a helper polypeptide is a DNA (e.g., a cDNA) oligonucleotide. In some embodiments, an oligonucleotide comprising a sequence that encodes a helper polypeptide is an RNA (e.g., mRNA) oligonucleotide.

In some embodiments, an oligonucleotide comprising a payload sequence is a synthetic oligonucleotide. In some embodiments, an oligonucleotide comprising a payload sequence is a DNA (e.g., a cDNA) oligonucleotide. In some embodiments, an oligonucleotide comprising a payload sequence is an RNA (e.g., mRNA) oligonucleotide.

In some embodiments, a nucleic acid expression system includes a synthetic DNA oligonucleotide comprising a payload sequence and at least one mRNA oligonucleotide sequence that encodes a helper polypeptide for enhancing persistence or uptake of the oligonucleotide comprising a payload sequence in a target cell.

In some embodiments, a helper polypeptide is or comprises a DNA mimic polypeptide. In some embodiments, a DNA mimic polypeptide is selected from any one of Ocr, ArdA, NuiA, HI1450, DMP12, MfpA, Am, Gam and/or variants thereof. In some embodiments, a DNA mimicking polypeptide is from bacteriophage. In some embodiments, a helper polypeptide is a fully engineered DNA mimic.

In some embodiments, a helper polypeptide is or comprises a viral modulator of innate immunity. Viral modulator of innate immunity include, for example, vIRF1, ORF52/KicGAS, PLP2-TM, PLP2, US11 and/or variants thereof.

In some embodiments, a helper polypeptide is or comprises a synthetic cell surface receptor polypeptide. Synthetic cell surface receptor polypeptides include, for example, TVA-EGF, H-EGF, H-IGF1 and/or variants thereof.

In some embodiments, (1) an oligonucleotide comprising a payload sequence and at least one (2)(a) oligonucleotide comprising a sequence that encodes a helper polypeptide or (2)(b) composition that delivers a helper polypeptide are administered sequentially. In some embodiments, (1) an oligonucleotide comprising a payload sequence and at least one (2)(a) oligonucleotide comprising a sequence that encodes a helper polypeptide or (2)(b) composition that delivers a helper polypeptide are administered concurrently.

In some embodiments provided are compositions that include the elements of a nucleic acid expression system as described herein. In some embodiments, a composition is a pharmaceutical composition. In some embodiments provided are pharmaceutical compositions that include the elements of a nucleic acid expression system as described herein.

In some embodiments provided are cells that include the elements of a nucleic acid expression system as described herein.

In some embodiments, provided are methods for enhancing expression of an oligonucleotide in a target cell, the method including: administering an oligonucleotide comprising a payload sequence; and administering at least one oligonucleotide sequences comprising a sequence that encodes a helper polypeptide.

In some embodiments, provided are methods for enhancing expression of an oligonucleotide in a target cell, the method including: administering an oligonucleotide comprising a payload sequence; and administering at least one RNA (e.g., a mRNA) oligonucleotide sequence that encodes a helper polypeptide.

In some embodiments, an oligonucleotide comprising a sequence that encodes a helper polypeptide is a synthetic oligonucleotide. In some embodiments, an oligonucleotide comprising a sequence that encodes a helper polypeptide is a DNA (e.g., cDNA) oligonucleotide. In some embodiments, an oligonucleotide comprising a sequence that encodes a helper polypeptide is an RNA (e.g., a mRNA) oligonucleotide.

In some embodiments, an oligonucleotide comprising a payload sequence is a synthetic oligonucleotide. In some embodiments, an oligonucleotide comprising a payload sequence is a DNA (e.g., cDNA) oligonucleotide. In some embodiments, an oligonucleotide comprising a payload sequence is an RNA (e.g., a mRNA) oligonucleotide. In some embodiments, an oligonucleotide comprising a payload sequence comprises homology arms.

In some embodiments, provided are methods for enhancing expression of an oligonucleotide in a target cell, the method including: administering an oligonucleotide comprising a payload sequence; and administering a composition that delivers a helper polypeptide.

In some embodiments, provided are methods for enhancing expression of an oligonucleotide in a target cell, where the method also includes an oligonucleotide encoding a targeted nuclease. In some embodiments, an oligonucleotide encoding a targeted nuclease is a DNA oligonucleotide. In some embodiments, an oligonucleotide encoding a targeted nuclease is a mRNA oligonucleotide. In some embodiments, a targeted nuclease is a zinc-finger nuclease (ZFN), TAL effector domain nuclease (TALEN), or an engineered CRISPR/Cas9 system.

In some embodiments, a helper polypeptide is or comprises a NLS polypeptide. In some embodiments, a NLS polypeptide is an SV40 NLS or variant thereof. In some embodiments, a NLS polypeptide is from EGL-13, c-Myc, NLP or TUS.

In some embodiments, a NLS polypeptide is operatively connected to a DNA-binding domain (DBD) polypeptide. In some embodiments, a DBD polypeptide is not regulated by a small molecule. In some embodiments, a DBD is or comprises a Cro repressor or a catalytically-inactive meganuclease variant. In some embodiments, a DBD polypeptide is a synthetic DBD. In some embodiments, a DBD is or comprises a zinc finger, a TAL domain, or a catalytically-inactive Cas9. In some embodiments, a DBD polypeptide is a non-specific DBD. In some embodiments, a DBD is or comprises Sso7d, H-NS, HU-1, HU-2, p6 of ϕ29, A104R of ASFV, dsp, TmHU, HPhA, or HCcp3.

In some embodiments, a NLS polypeptide is fused with a DBD. In some embodiments, a NLS polypeptide and DBD are separate polypeptides that can join to form a complex (e.g., dimerize). In some embodiments, a NLS polypeptide and a DBD dimerize through inducible dimerization domains. Exemplary inducible dimerization domains include a rapamycin-inducible FRB/FKBP pair.

In some embodiments, a helper polypeptide is or comprises a DNA mimic polypeptide. In some embodiments, a DNA mimic polypeptide is selected from any one of Ocr, ArdA, NuiA, HI1450, DMP12, MfpA, Am, Gam and/or variants thereof. In some embodiments, a DNA mimicking polypeptide is from bacteriophage. In some embodiments, a helper polypeptide is a fully engineered DNA mimic.

In some embodiments, a helper polypeptide is or comprises a viral modulator of innate immunity. Viral modulators of innate immunity include, for example, vIRF1, ORF52/KicGAS, PLP2-TM, PLP2, US11 and/or variants thereof.

In some embodiments, a helper polypeptide is or comprises a synthetic cell surface receptor polypeptide. Synthetic cell surface receptor polypeptides include, for example, TVA-EGF, H-EGF, H-IGF1 and/or variants thereof.

In some embodiments, provided are methods for increasing nuclear localization of an oligonucleotide comprising, the method including: administering an oligonucleotide comprising a payload sequence; and administering an oligonucleotide sequence that encodes a helper polypeptide for enhancing nuclear import of the oligonucleotide comprising a payload sequence in a target cell. In some embodiments, a helper polypeptide is or comprises a NLS polypeptide.

In some embodiments, provided are methods for increasing nuclear localization of an oligonucleotide comprising, the method including: administering a DNA oligonucleotide comprising a payload sequence; and administering an at least one mRNA oligonucleotide sequence that encodes a helper polypeptide for enhancing nuclear import of the oligonucleotide comprising a payload sequence in a target cell. In some embodiments, a helper polypeptide is or comprises a NLS polypeptide.

In some embodiments, provided methods include: administering an oligonucleotide comprising a payload sequence; and administering an at least one oligonucleotide sequence that encodes a helper polypeptide comprising a nuclear localization signal (NLS) polypeptide, and an oligonucleotide encoding a DNA-binding domain (DBD) polypeptide.

In some embodiments, a NLS polypeptide is an SV40 NLS or variant thereof. In some embodiments, a NLS polypeptide is from EGL-13, c-Myc, NLP or TUS.

In some embodiments, a NLS polypeptide is operatively connected to a DNA-binding domain (DBD) polypeptide. In some embodiments, a DBD polypeptide is not regulated by a small molecule. In some embodiments, a DBD is or comprises a Cro repressor or a catalytically-inactive meganuclease variant. In some embodiments, a DBD polypeptide is a synthetic DBD. In some embodiments, a DBD is or comprises a zinc finger, a TAL domain, or a catalytically-inactive Cas9. In some embodiments, a DBD polypeptide is a non-specific DBD. In some embodiments, a DBD is or comprises Sso7d, H-NS, HU-1, HU-2, p6 of ϕ29, A104R of ASFV, dsp, TmHU, HPhA, or HCcp3.

In some embodiments, a NLS polypeptide is fused with a DBD. In some embodiments, a NLS polypeptide and DBD are separate polypeptides that can join to form a complex (e.g., dimerize). In some embodiments, a NLS polypeptide and a DBD dimerize through inducible dimerization domains. Exemplary inducible dimerization domains include a rapamycin-inducible FRB/FKBP pair.

In some embodiments, provided are methods for enhancing persistence or uptake of an oligonucleotide comprising, the method including: administering an oligonucleotide comprising a payload sequence; and administering an oligonucleotide sequence that encodes a helper polypeptide for enhancing persistence or uptake of the oligonucleotide comprising a payload sequence in a target cell.

In some embodiments, provided are methods for enhancing persistence or uptake of an oligonucleotide comprising, the method including: administering a DNA oligonucleotide comprising a payload sequence; and administering an at least one mRNA oligonucleotide sequence that encodes a helper polypeptide for enhancing persistence or uptake of the oligonucleotide comprising a payload sequence in a target cell.

In some embodiments, an oligonucleotide comprising a payload sequence and the at least one oligonucleotide comprising a sequence that encodes a helper polypeptide are administered sequentially. In some embodiments, an oligonucleotide comprising a payload sequence and the at least one oligonucleotide comprising a sequence that encodes a helper polypeptide are administered concurrently. In some embodiments, an oligonucleotide comprising a payload sequence and at least one oligonucleotide comprising a sequence that encodes a helper polypeptide are part of a vector.

In some embodiments, an oligonucleotide comprising a payload sequence, at least one oligonucleotide comprising a sequence that encodes a helper polypeptide or composition that delivers a helper polypeptide, and/or an oligonucleotide encoding a targeted nuclease are administered separately. In some embodiments, an oligonucleotide comprising a payload sequence, at least one oligonucleotide comprising a sequence that encodes a helper polypeptide or composition that delivers a helper polypeptide, and/or an oligonucleotide encoding a targeted nuclease are administered concurrently.

In some embodiments, a helper polypeptide is or comprises a DNA mimic polypeptide. In some embodiments, a DNA mimic polypeptide is selected from any one of Ocr, ArdA, NuiA, HI1450, DMP12, MfpA, Am, Gam and/or variants thereof. In some embodiments, a DNA mimicking polypeptide is from bacteriophage. In some embodiments, a helper polypeptide is a fully engineered DNA mimic.

In some embodiments, a helper polypeptide is or comprises a viral modulator of innate immunity. Viral modulator of innate immunity include, for example, vIRF1, ORF52/KicGAS, PLP2-TM, PLP2, US11 and/or variants thereof.

In some embodiments, a helper polypeptide is or comprises a synthetic cell surface receptor polypeptide. Synthetic cell surface receptor polypeptides include, for example, TVA-EGF, H-EGF, H-IGF1 and/or variants thereof.

### BRIEF DESCRIPTION OF THE DRAWING

FIG. 1 depicts expression of a model DNA oligonucleotide when co-transfected with an oligonucleotide construct that encodes a TetR, a NLS polypeptide-TetR or a control DNA. Luciferase luminescence (y-axis) indicates expression of model DNA oligonucleotide.

FIG. 2 depicts expression of a model DNA oligonucleotide that encodes a luciferase reporter when transfected into cells that have previously been transfected with an oligonucleotide encoding TetR, NLS-TetR, TmHU, or control DNA. Panels A-E show luciferase luminescence (y-axis) normalized to that of a no plasmid control, at five different time periods: (A) 0-16 h, (B) 22-40 h, (C) 44-111 h, (D) 111-163 h, and (E) 163-231 h.

FIG. 3 depicts expression of a model DNA oligonucleotide that encodes a luciferase reporter when co-transfected into cells with an oligonucleotide encoding candidate DNA mimic polypeptides (EKC62359, EKC78842) or control DNA.

FIG. 4 depicts expression of a model DNA oligonucleotide that encodes a luciferase reporter when co-transfected into cells with a RNA oligonucleotide comprising a sequence that encodes a DNA mimic polypeptide or a negative control sequence. *N=2 replicate transfections, * indicates p<0.05 and **=p<0.005 a via a two-sample two-tailed Student's t-test.

FIG. 5 depicts expression of a model payload sequence when an RNA oligonucleotide comprising a payload sequence is delivered to target cells in the presence of various amounts of an RNA oligonucleotide comprising a sequence that encodes an immunomodulatory polypeptide (e.g., a US11 polypeptide). Luciferase luminescence (y-axis) indicates expression of a model payload sequence (e.g., luc2).

FIGS. 6A-6C depict viability of cells upon repeated transfections with an RNA oligonucleotide comprising a control sequence with or without an RNA oligonucleotide comprising a sequence that encodes a US11 polypeptide. FIG. 6A shows cell viability after a first transfection with RNA oligonucleotides as indicated according to one embodiment described herein. FIG. 6B shows cell viability after a second transfection with RNA oligonucleotide as indicated according to one embodiment described herein. FIG. 6C shows cell viability after a third transfection with RNA oligonucleotides as indicated according to one embodiment described herein.

FIGS. 7A-7C depict viability of cells upon repeated transfections with an exemplary RNA oligonucleotide comprising a control sequence with or without an exemplary RNA oligonucleotide comprising a sequence that encodes a US11 polypeptide. FIG. 7A shows cell viability after a first transfection with RNA oligonucleotides as indicated according to another embodiment described herein. FIG. 7B shows cell viability after a second transfection with RNA oligonucleotide as indicated according to another embodiment described herein. FIG. 7C shows cell viability after a third transfection with RNA oligonucleotides as indicated according to another embodiment described herein.

### CERTAIN DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) where ranges are provided, endpoints are included.

About or approximately: As used herein, the terms “about” and “approximately,” when used herein in reference to a value, refers to a value that is similar, in context to the referenced value. In general, those skilled in the art, familiar with the context, will appreciate the relevant degree of variance encompassed by “about” or “approximately” in that context. For example, in some embodiments, the term “about” or “approximately” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to administration of a composition to a subject to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g, a plurality of doses separated in time) and/or periodic (e.g., individual doses separated by a common period of time) dosing. In some embodiments, administration may involve continuous dosing (e.g., perfusion) for at least a selected period of time.

Amplification: As used herein, the term “amplification,” when used in reference to polynucleotides, refers to a method that increases the representation in a population of a specific nucleotide sequence (e.g., from a template polynucleotide) in a sample by producing multiple (i.e., at least 2) copies of the desired nucleotide sequence. Methods for nucleic acid amplification are known in the art and include, but are not limited to, polymerase chain reaction (PCR) and ligase chain reaction (LCR) (i.e., a reaction using both a DNA polymerase and a DNA ligase, as well as two probes that are ligated together to form a single probe during LCR). Variants of standard PCR or LCR reactions can also be used. A “copy” or “amplicon” does not necessarily have perfect sequence complementarity or identity to the nucleotide sequence in the template polynucleotide. Unless otherwise specified, one or more copies can comprise one or more mutant copies, i.e., copies containing one or more mutations (“mutant copies”) as compared to the nucleotide sequence in the template polynucleotide. Mutant copies can comprise mutations in one or more bases. For example, for template polynucleotides that comprise a coding region with a plurality of codons, mutant copies can comprise mutations in one or more than one codon and within each codon, there can be mutations in one, two, or all three nucleotides of the codon. In general, “mutations” will be understood to include substitutions, insertions or deletions relative to the template polynucleotide.

Co-delivery: As used herein, the term “co-delivery” refers to use of both an oligonucleotide (e.g., DNA or RNA oligonucleotide) comprising a payload sequence and an oligonucleotide (e.g., DNA or RNA oligonucleotide) comprising a sequence that encodes a helper polypeptide to deliver a payload sequence into a target cell (e.g., a cell cultured in vitro or ex vivo, or a cell present in tissue of a subject). The combined use of an oligonucleotide comprising a payload sequence and an oligonucleotide comprising a sequence that encodes a helper polypeptide may be performed concurrently or separately (e.g., sequentially in any order). In some embodiments of a pharmaceutical composition described herein, both an oligonucleotide comprising a payload sequence and an oligonucleotide comprising a sequence that encodes a helper polypeptide may be combined in one pharmaceutically-acceptable carrier, or they may be placed in separate carriers and delivered to a target cell (e.g., a cell cultured in vitro or ex vivo, or a cell present in tissue of a subject) or administered to a subject at different times. Each of these situations is contemplated as falling within the meaning of “co-delivery” or “co-administration” or “combination,” provided that both an oligonucleotide comprising a payload sequence and an oligonucleotide comprising a sequence that encodes a helper polypeptide are delivered or administered sufficiently close in time that there is at least some temporal overlap in biological effect(s) generated by both oligonucleotides on a target cell or a subject being treated.

Complementary: As used herein, the term “complementary” refers to nucleotides or nucleotide sequences that base-pair according to the standard Watson-Crick complementary rules (adenine “A” base pairs with thymine “T”, and guanine “G” base pairs with cytosine “C”). Nucleotide sequences that are “100% complementary” or which exhibit “100% complementarity” are nucleotide sequences which base-pair with one another across the entirety of at least one of the two nucleotide sequences. An oligonucleotide can be “100% complementary” to a template polynucleotide that is longer than the oligonucleotide (i.e., the oligonucleotide is “100% complementary” to the template polynucleotide if the entire sequence of the oligonucleotide base-pairs with a portion of the template polynucleotide). However, nucleic acid sequences that are “complementary” need not be 100% complementary. Generally, the term “complementary” with respect to two or more nucleic acid sequences refers to there being sufficient complementarity across the two nucleic acid sequences such that they hybridize in stringent conditions and/or at temperatures used during annealing phases of amplification methods, e.g., PCR or LCR.

Delivery/contacting: As used interchangeably herein, the term “delivery,” “delivering,” or “contacting” refers to introduction of an oligonucleotide (e.g., a DNA or RNA oligonucleotide comprising a payload sequence or comprising a sequence encoding a helper polypeptide) into a target cell (e.g., cytosol of a target cell, which can be, for example, a cell cultured in vitro or ex vivo, or a cell present in tissue of a subject). In some embodiments, a target cell can be cultured in vitro or ex vivo. In some embodiments, a target cell can be present in a subject, e.g., in a tissue of a subject (in vivo). Methods of introducing an oligonucleotide into a target cell can vary with in vitro, ex vivo, or in vivo applications. In some embodiments, an oligonucleotide can be introduced into a target cell in a cell culture by in vitro transfection. In some embodiments, an oligonucleotide can be introduced into a target cell (e.g., a cell cultured in vitro or ex vivo, or a cell present in tissue of a subject) via delivery vehicles (e.g., nanoparticles, liposomes, and/or complexation with a cell-penetrating agent). In some embodiments, an oligonucleotide can be introduced into a target cell in a subject by administering an oligonucleotide to a subject.

DNA oligonucleotide: As used herein, the term “DNA oligonucleotide” refers to an oligonucleotide of deoxyribonucleotides. In some embodiments, a DNA oligonucleotide is single stranded. In some embodiments, a DNA oligonucleotide is double stranded. In some embodiments, a DNA oligonucleotide comprises both single and double stranded portions. In some embodiments, a DNA oligonucleotide can comprise a backbone structure as described in the definition of “Nucleic acid/Oligonucleotide” below. In some embodiments, a DNA oligonucleotide is a synthetic DNA oligonucleotide exogenously introduced into a cell or a subject for expressing a payload sequence of interest.

Expression: As used herein, “expression” of oligonucleotide sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Homology: As used herein, the term “homology” or “homolog” refers to the overall relatedness between oligonucleotide molecules (e.g., DNA molecules and/or RNA molecules) and/or between polypeptide molecules. In some embodiments, oligonucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, oligonucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Hybridized: As used herein, the term “hybridize” or “hybridization” refers to a process where two strands in a double-stranded polynucleotide, or two portions of single-stranded polynucleotide, anneal to each other under appropriately stringent conditions. The phrase “is capable is hybridizing to” refers to the ability of two nucleotide sequences to hybridize to each other under typical hybridization conditions (e.g., in the context of a typical amplification reaction, “hybridize” would refer to the interaction of two complementary nucleotide sequences during the annealing phase). As understood by one of ordinary skill in the art, nucleotide sequences need not have perfect sequence complementarity to hybridize with one another. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, et al. 1994, Current Protocols in Molecular Biology. John Wiley & Sons, Secaucus, N.J.

Identity: As used herein, the term “identity” refers to the overall relatedness between oligonucleotide molecules (e.g., DNA molecules and/or RNA molecules) and/or between polypeptide molecules. In some embodiments, oligonucleotide molecules (e.g., DNA molecules and/or RNA molecules) and/or between polypeptide molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Label: The term “label” as used herein refers to any element, molecule, functional group, compound, fragment or moiety that is detectable. In some embodiments, a label is provided or utilized alone. In some embodiments, a label is provided and/or utilized in association with (e.g., joined to) another agent. Examples of labels include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinium esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Non-specific toxicity: In context of introduction of an oligonucleotide, e.g., an oligonucleotide comprising a payload sequence, into a target cell, the term “non-specific toxicity” refers to cell toxicity induced by an oligonucleotide independent of a function and/or activity of a payload sequence. For example, when an oligonucleotide comprising a non-cytotoxic payload sequence causes comparable cell death (an exemplary indicator of cell toxicity) to that caused by an oligonucleotide comprising a cytotoxic payload sequence, the cell death (or cell toxicity) is nonspecific because it is independent of the cytotoxic nature of a payload sequence. In some embodiments, “non-specific toxicity” also refers to cell toxicity induced in any cells including, e.g., both target and non-target cells (e.g., normal healthy cells), rather than induced in target cells only.

Nucleic acid/Oligonucleotide: As used herein, the terms “nucleic acid” and “oligonucleotide” are used interchangeably, and refer to a polymer of at least 3 nucleotides or more. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleotide: As used herein, the term “nucleotide” refers to its art-recognized meaning. When a number of nucleotides is used as an indication of a distance, e.g., between elements on a nucleic acid, a certain number of nucleotides refers to the number of nucleotides on a single strand of the nucleic acid between the elements, regardless of whether the nucleic acid is double-stranded, single-stranded, or partly double-stranded and partly single-stranded. Similarly, when a number of nucleotides is used as an indication of size, e.g., of a nucleic acid element or an oligonucleotide, a certain number of nucleotides refers to the number of nucleotides on a single strand, e.g., of a nucleic acid element or an oligonucleotide.

Polypeptide: The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” may be used to refer to the multiple polypeptides that are physically associated and function together as the discrete unit. In some embodiments, proteins may include moieties other than amino acids (e.g., may be glycoproteins, proteoglycans, etc.) and/or may be otherwise processed or modified. Those of ordinary skill in the art will appreciate that in some embodiments the term “protein” may refer to a complete polypeptide chain as produced by a cell (e.g., with or without a signal sequence), and/or to a form that is active within a cell (e.g., a truncated or complexed form). In some embodiments where a protein is comprised of multiple polypeptide chains, such chains may be covalently associated with one another, for example by one or more disulfide bonds, or may be associated by other means.

Primer: As used herein, the term “primer” is interchangeable with “oligonucleotide primer” and is used herein to refer to an oligonucleotide that acts as a point of initiation of synthesis of a primer extension product when hybridized to a template polynucleotide, when placed under suitable conditions (e.g., buffer, salt, temperature and pH), in the presence of nucleotides and an agent for nucleic acid polymerization (e.g., a DNA-dependent or RNA-dependent polymerase). The primer is preferably single-stranded for maximum efficiency in amplification, but may alternatively be double-stranded. If double-stranded, the primer may first be treated (e.g., denatured) to allow separation of its strands before being used to prepare extension products. Such a denaturation step is typically performed using heat, but may alternatively be carried out using alkali, followed by neutralization. A typical primer comprises a sequence of about 10 to about 50, e.g., about 20 to about 40 nucleotides that is complementary to a sequence in a template polynucleotide.

Recombination: As used herein, the term “recombination” refers to a process of exchange of genetic information between two polynucleotides. For the purposes of this disclosure, “homologous recombination” (HR) refers to a specialized form of such exchange that takes place, for example, during repair of nicks and/or double-strand breaks DNA (e.g., genomic DNA). Typically a payload sequence to be incorporated has nucleotide sequence homology to a region of the “target” molecule (i.e., nucleic acid molecule that experienced the nick and/or double-strand break). For example, a payload sequence can include homology arms that hybridize with one or more genomic sequences that flank a cleavage site. This often leads to the transfer of genetic information from payload oligonucleotide to the target molecule (e.g., genomic DNA). Without wishing to be bound by any particular theory, such transfer can involve mismatch correction of heteroduplex DNA that forms between a broken target and an oligonucleotide comprising a payload, and/or “synthesis-dependent strand annealing,” in which an oligonucleotide comprising a payload is used to resynthesize genetic information that will become part of the target, and/or related processes. Such specialized HR often results in an alteration of the sequence of a target molecule such that part or all of the sequence of a payload sequence is incorporated into the target polynucleotide.

RNA oligonucleotide: As used herein, the term “RNA oligonucleotide” refers to an oligonucleotide of ribonucleotides. In some embodiments, an RNA oligonucleotide is single stranded. In some embodiments, an RNA oligonucleotide is double stranded. In some embodiments, an RNA oligonucleotide comprises both single and double stranded portions. In some embodiments, an RNA oligonucleotide can comprise a backbone structure as described in the definition of “Nucleic acid/Oligonucleotide” above. An RNA oligonucleotide can be a regulatory RNA (e.g., siRNA, microRNA, etc.), or a messenger RNA (mRNA) oligonucleotide. In some embodiments where an RNA oligonucleotide is a mRNA oligonucleotide, an RNA oligonucleotide typically comprises at its 3′ end a poly(A) region. In some embodiments where an RNA oligonucleotide is a mRNA oligonucleotide, an RNA oligonucleotide typically comprises at its 5′ end an art-recognized cap structure, e.g., for recognizing and attachment of a mRNA to a ribosome to initiate translation.

Target cell: As used herein, the term “target cell” refers to a cell that receives an oligonucleotide comprising a payload sequence and/or an oligonucleotide comprising a sequence that encodes a helper polypeptide. In some embodiments, a target cell is a cell that has been contacted, e.g., at least once (e.g., at least twice or more), with an oligonucleotide. For example, in some embodiments, a target cell is a cell that has been contacted, e.g., at least once (e.g., at least twice or more), with an oligonucleotide comprising a payload sequence. In some embodiments, a target cell is a cell that has been contacted, e.g., at least once (e.g., at least twice or more), with an oligonucleotide comprising a sequence that encodes a helper polypeptide (e.g., ones described herein). In some embodiments, a target cell is a cell that has been contacted, e.g., at least once (e.g., at least twice or more), with an oligonucleotide comprising a payload sequence and an oligonucleotide comprising a sequence that encodes a helper polypeptide (e.g., ones described herein). A target cell in any aspects described herein can be present in a cell culture (in vivo or ex vivo) or in a tissue or organ of a subject (in vivo). A target cell in any aspects described herein can be a wild-type cell, a normal cell, a diseased cell, or a transgenic cell. In some embodiments, a target cell is an eukaryotic cell (e.g., a mammalian cell). In some embodiments, a target cell is a human cell.

Target Site: As used herein, the term “target site” refers to a nucleic acid sequence that defines a portion of a nucleic acid to which a binding molecule will bind, provided sufficient conditions for binding exist.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Subject: As used herein, the term “subject” refers an organism, typically a mammal (e.g., a human). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered. In some embodiments, a subject is an individual (e.g., a human) who has undergone an RNA oligonucleotide therapy or a gene therapy at least once or more. In some embodiments, a subject is an individual (e.g., a human) who is undergoing an RNA oligonucleotide therapy or a gene therapy.

Variant: As used herein, the term “variant” refers to a polypeptide that is derived from a reference polypeptide. Typically, a variant differs from a reference polypeptide by at least one or more amino acid residues, which may have been added to or deleted from either or both the N-terminal or C-terminal end of a reference polypeptide; and/or inserted at or deleted from one or more sites within the sequence of a reference polypeptide; and/or substituted with one or more amino acid residues within, or at either or both ends of the amino acid sequence of a reference polypeptide.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2nd ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

Modem gene therapy approaches are, with strikingly few exceptions, based on one of three approaches: engineered viruses, non-viral DNA vectors, or mRNAs. Unfortunately, each strategy has large technological limitations. For example, viruses can elicit a potent adaptive immune response, making them ineffective after only a small number of lifetime exposures (Nayak & Herzog (2009) Gene Therapy, 17:295-304, which is incorporated herein by reference in its entirety). In contrast, non-viral DNA vectors can enable gene delivery without major immunological issues, but often suffer from poor in vivo delivery efficiencies. mRNA-based gene expression systems can achieve high expression levels, but often exhibit rapidly decaying kinetics. The present disclosure provides, among other things, a next-generation gene delivery platform that combines beneficial aspects of these different strategies.

The present disclosure encompasses the recognition that non-viral DNA vectors have a capacity for long-term transgene expression and low immunogenicity. However, as noted above, these vectors have low transfection efficiencies that can only be overcome with the use of harsh physical methods such as electroporation (Roche et al. (2011) American Journal of Physiology—Cell Physiology, 301: C1239-C1250, which is incorporated herein by reference in its entirety). The present disclosure further encompasses the recognition that use of helper polypeptides may remedy some of the deficiencies of non-viral DNA vectors.

The present disclosure is based, at least in part, on an unexpected discovery that co-delivery (e.g., to a subject or target cell) of an oligonucleotide (e.g., DNA or RNA) oligonucleotide comprising a sequence that encodes a helper polypeptide (e.g., a nuclear localization signal (NLS) polypeptide, a DNA mimic polypeptide, an immunomodulatory polypeptide, and/or a synthetic cell surface receptor polypeptide) with an oligonucleotide (e.g., DNA or RNA) comprising a sequence that encodes a payload results in increased expression of the payload.

The present disclosure also recognizes that an immunomodulatory polypeptide (e.g., a modulator of innate immunity such as a US11 polypeptide) may reduce innate immunity-triggered suppression of protein translation and/or RNA degradation. A reduction in innate immunity-triggered suppression of protein translation and/or RNA degradation can, in turn, improve expression of a target payload from a co-delivered RNA (e.g., mRNA) oligonucleotide in target cells. The present disclosure also encompasses the surprising discovery that delivery of an RNA (e.g., mRNA) oligonucleotide comprising a sequence that encodes an US11 polypeptide to, e.g., a subject or target cell, can reduce non-specific toxicity induced by RNA (e.g., mRNA) oligonucleotides. The present disclosure also encompasses the surprising discovery that co-delivery of an RNA (e.g., mRNA) oligonucleotide comprising a sequence that encodes an immunomodulatory polypeptide (e.g., a modulator of innate immunity such as a US11 polypeptide) with an RNA (e.g., mRNA) oligonucleotide comprising a payload sequence can reduce non-specific toxicity induced by RNA (e.g., mRNA) oligonucleotides, e.g., the RNA (e.g., mRNA) encoding a payload. The present disclosure also provides compositions including an RNA (e.g., mRNA) oligonucleotide comprising a sequence that encodes an immunomodulatory polypeptide (e.g., a modulator of innate immunity such as a US11 polypeptide) that can be delivered more than once to a subject or target cells, e.g., to improve expression and/or activity of, e.g., an RNA (e.g., mRNA) oligonucleotide comprising a payload sequence without substantially increasing non-specific toxicity induced by RNA oligonucleotides.

Accordingly, the present disclosure provides nucleic acid expression systems and compositions for delivery of an oligonucleotide (e.g., DNA or RNA) comprising a payload sequence with an oligonucleotide (e.g., DNA or RNA) comprising a sequence that encodes a helper polypeptide (e.g., ones described herein). In one aspect, the present disclosure provides nucleic acid expression systems that employ synthetic versions of viral delivery strategies to enhance the efficiency of transfection of oligonucleotides comprising a payload sequence. Methods for using nucleic acid expression systems and compositions are also provided herein.

### I. Nucleic Acid Expression Systems

The present disclosure provides nucleic acid expression systems for expression of oligonucleotides in cells. Such nucleic acid expression systems may be used, for example, as part of a gene therapy. The present disclosure provides the insight that efficacy of a gene therapy may be enhanced (e.g., expression, nuclear import, persistence or uptake of a payload oligonucleotide may be increased in a target cell) by co-expression of one or more helper proteins.

In some embodiments, a nucleic acid expression system includes an oligonucleotide comprising a payload sequence and at least one oligonucleotide comprising a sequence that encodes a helper polypeptide, which confers one or more of the following characteristics: (i) enhancing expression and/or activity of an oligonucleotide comprising a payload sequence in a target cell; (ii) enhancing nuclear import of an oligonucleotide comprising a payload sequence in a target cell; (iii) enhancing persistence or uptake of an oligonucleotide comprising a payload sequence in a target cell; (iv) enhancing the viability of a target cell upon contacting with an oligonucleotide comprising a payload sequence; and (v) reducing non-specific toxicity induced in a target cell by an oligonucleotide comprising a payload sequence.

In some embodiments, a nucleic acid expression system includes a oligonucleotide comprising a payload sequence and a composition that delivers at least one helper polypeptide.

In some embodiments, a nucleic acid expression system includes at least one oligonucleotide comprising a payload sequence as described herein and least one oligonucleotide comprising a sequence that encodes a helper polypeptide as described herein. In some embodiments, a nucleic acid expression system includes 1, 2, 3, 4, 5, 6, 7, 8, 9, or 10 oligonucleotides comprising a payload sequence. In some embodiments, a nucleic acid expression system includes at least 2, 3, 4, 5, 6, 7, 8, 9, or 10 oligonucleotides that comprising a sequence that encodes a helper polypeptide. In some embodiments, helper polypeptides in nucleic acid expression systems described herein can be or include a nuclear localization signal (NLS) polypeptide, a DNA mimic polypeptide, an immunomodulatory polypeptide (e.g., a modulator of innate immunity), or a synthetic cell surface receptor polypeptide.

The present disclosure further provides expression systems that leverage short-term high-level mRNA-based protein expression to enhance the efficiency of transfection of oligonucleotides comprising a payload sequence. In some embodiments, a nucleic acid expression system includes a synthetic DNA oligonucleotide comprising a payload sequence and at least one mRNA oligonucleotide that encodes a helper polypeptide. In some embodiments, at least one mRNA oligonucleotide comprising a sequence that encodes a helper polypeptide for achieving one or more of the following characteristics: (i) enhancing expression and/or activity of an oligonucleotide comprising a payload sequence in a target cell; (ii) enhancing nuclear import of an oligonucleotide comprising a payload sequence in a target cell; (iii) enhancing persistence or uptake of an oligonucleotide comprising a payload sequence in a target cell; (iv) enhancing the viability of a target cell upon contacting with an oligonucleotide comprising a payload sequence; and (v) reducing non-specific toxicity induced in a target cell by an oligonucleotide comprising a payload sequence.

In some embodiments involving any nucleic acid expression systems described herein, an oligonucleotide comprising a payload sequence is a DNA oligonucleotide and an oligonucleotide comprising a sequence that encodes a helper polypeptide (e.g., ones described herein) is a DNA oligonucleotide. In some embodiments involving any nucleic acid expression systems described herein, an oligonucleotide comprising a payload sequence is a DNA oligonucleotide and an oligonucleotide comprising a sequence that encodes a helper polypeptide (e.g., ones described herein) is an RNA (e.g., mRNA) oligonucleotide. In some embodiments involving any nucleic acid expression systems described herein, an oligonucleotide comprising a payload sequence is an RNA (e.g., mRNA) oligonucleotide and an oligonucleotide comprising a sequence that encodes a helper polypeptide (e.g., ones described herein) is an RNA (e.g., mRNA) oligonucleotide.

In some embodiments, oligonucleotides (e.g., comprising a payload and/or comprising a sequence that encodes a helper polypeptide) of any aspects described herein are synthetic oligonucleotides. For example, in some embodiments, a DNA oligonucleotide comprising a payload sequence is a synthetic DNA oligonucleotide. In some embodiments, a DNA oligonucleotide comprising a sequence that encodes a helper polypeptide is a synthetic DNA oligonucleotide. Synthetic DNA oligonucleotides can be produced by methods known in the art, e.g., by chemical synthesis.

In some embodiments, an RNA oligonucleotide comprising a payload sequence is a synthetic RNA oligonucleotide. In some embodiments, an RNA oligonucleotide comprising a sequence that encodes a helper polypeptide is a synthetic RNA oligonucleotide. Synthetic RNA oligonucleotides can be produced by any methods known in the art. For example, in some embodiments where synthetic RNA oligonucleotides are synthetic mRNA oligonucleotides, they can be produced, e.g., by in vitro transcription of a cDNA template, typically plasmid DNA (pDNA), using an RNA polymerase, e.g., a bacteriophage RNA polymerase.

Helper Polypeptides

Use of helper polypeptides to facilitate nuclear localization is a viral strategy for transduction of non-replicating cells (Citovsky et al. (1994) Proc. National Acad. Sci., 91: 3210-3214; Matreyek & Engelman (2013) Viruses, 5: 2483-2511; Kobiler et al. (2014) Nucleus, 3: 526-539, the contents of each of which are incorporated herein by reference in their entirety). Other common viral strategies include suppression of innate immunity (Katze et al. (2008) Immunol., 8: 644-654, which is incorporated herein by reference in its entirety), inhibition of cellular nucleases (Wang et al. (2014) Biochemistry, 53: 2865-2874, which is incorporated herein by reference in its entirety), and the production of ligand and/or receptor mimics to stimulate uptake of viral particles (Alcami (2003) Nat. Rev. Immunol., 3: 36-50, which is incorporated herein by reference in its entirety).

In one aspect, the present disclosure provides the insight that co-delivery of helper polypeptides that employ viral mechanisms with payload oligonucleotides can provide beneficial effects (e.g., ones described herein such as enhancing expression, nuclear import, persistence and/or uptake of a payload oligonucleotide). In some embodiments, a viral mechanism includes one or more of: increasing nuclear localization, suppressing innate immunity, reducing degradation of payload oligonucleotides and increasing uptake of a payload oligonucleotide. In some embodiments, a helper protein in the context of the present disclosure mimics a viral mechanism to enhance expression, nuclear import, persistence or uptake of a non-viral oligonucleotide. In some embodiments, a viral mechanism to enhance expression of a non-viral nucleotide includes one or more of increasing nuclear localization, increasing persistence of the oligonucleotide, and suppressing innate immunity.

In some embodiments, an oligonucleotide that encodes a helper polypeptide is a DNA oligonucleotide. In some embodiments, an oligonucleotide that encodes a helper polypeptide is a RNA oligonucleotide. In some certain embodiments, an oligonucleotide sequence that encodes a helper polypeptide is an mRNA oligonucleotide.

Nuclear Localization Signal and DNA Binding Domain Polypeptides

The present disclosure encompasses the recognition that that mRNA vectors may achieve higher efficiencies than DNA vectors under identical delivery conditions. Since mRNA can be expressed cytoplasmically while DNA requires nuclear localization, these observations indicate that lack of nuclear transport is the rate-limiting step in DNA delivery (Zou et al. (2010) International Journal of Pharmaceutics, 389(1): 232-243, which is incorporated herein by reference in its entirety). This rate limitation may particularly pronounced in non-dividing cells.

The present disclosure provides the insight that use of a nuclear localization signal (NLS) polypeptide, e.g., associated with a DNA binding domain (DBD) polypeptide, may facilitate nuclear transport of DNA oligonucleotides comprising a payload sequence into a cell nucleus.

In some embodiments, a helper polypeptide is or comprises a NLS polypeptide. In some embodiments, a NLS polypeptide is an simian virus 40 (SV40) NLS polypeptide or variant thereof. In some embodiments, a NLS polypeptide is from a EGL-13 polypeptide, a c-Myc polypeptide, a nucleoplasmin-like protein (NLP) polypeptide or a TUS (a DNA-binding polypeptide).

In some embodiments, a NLS polypeptide is operatively connected to a DNA-binding domain (DBD) polypeptide. In some embodiments, a DBD polypeptide is not regulated by a small molecule. In some embodiments, a DBD is or comprises a Cro repressor or a catalytically-inactive meganuclease variant. In some embodiments, a DBD polypeptide is a synthetic DBD polypeptide. In some embodiments, a DBD polypeptide is or comprises a zinc finger polypeptide, a TAL domain polypeptide, or a catalytically-inactive Cas9 polypeptide. In some embodiments, a DBD polypeptide is a non-specific DBD polypeptide. In some embodiments, a DBD polypeptide is or comprises Sso7d polypeptide, H-NS polypeptide, HU-1 polypeptide, HU-2 polypeptide, p6 polypeptide of 429, A104R polypeptide of ASFV, dsp polypeptide, TmHU polypeptide, HPhA polypeptide, or HCcp3 polypeptide.

In some embodiments, a NLS polypeptide is fused with a DBD polypeptide to form a fusion polypeptide. In some embodiments, a NLS polypeptide and a DBD polypeptide are separate polypeptides that can join to form a complex (e.g., by dimerization). In some embodiments, a NLS polypeptide and a DBD polypeptide can dimerize through inducible dimerization domains. Exemplary inducible dimerization domains include a rapamycin-inducible FRB/FKBP pair.

In some embodiments, delivery of a helper polypeptide that includes a NLS polypeptide increases nuclear localization of an oligonucleotide comprising a payload sequence in a target cell. For example, in some embodiments, delivery of a helper polypeptide that includes a NLS polypeptide increases nuclear localization of an oligonucleotide comprising a payload sequence in a target cell by at least about 30%, including, e.g., at least about 40%, at least about 50%, at least about 60%, at least about 70%, at least about 80%, at least about 90% or more, as compared to nuclear localization of an oligonucleotide comprising a payload sequence introduced into a target cell in the absence of a helper polypeptide. In some embodiments, delivery of a helper polypeptide that includes a NLS polypeptide increases expression/activity of an oligonucleotide comprising a payload sequence in a target cell. For example, in some embodiments, delivery of a helper polypeptide that includes a NLS polypeptide increases expression/activity of an oligonucleotide comprising a payload sequence in a target cell by at least about 30%, including, e.g., at least about 40%, at least about 50%, at least about 60%, at least about 70%, at least about 80%, at least about 90% or more, as compared to expression and/or activity of an oligonucleotide comprising a payload sequence introduced into a target cell in the absence of a helper polypeptide.

DNA Mimic Polypeptides

DNA in the cytoplasm is rapidly degraded by the nucleases cells that have evolved for defending against pathogens. For example, single- and double-stranded DNA microinjected into cytoplasms of various mammalian cell lines can be degraded within <90 minutes (Lechardeur et al. (1999) Gene Ther., 6, which is incorporated herein by reference in its entirety). The identities of the nucleases involved in this process remain poorly defined, making selective inhibition of specific enzymes difficult.

For successful protein expression from gene therapy vectors, the delivered DNA oligonucleotides must successfully transverse the cytoplasm and enter the nucleus. Without facilitated transport, nuclear entry happens via mass action, with approximately 1 in 10⁵-10⁶ DNA molecules spontaneously entering the nucleus (Utvik et al. (1999) Human gene therapy, 10(2), pp. 291-300, which is incorporated herein by reference in its entirety). Most of DNA oligonucleotides are degraded in the cytoplasm by a poorly defined set of endogenous nucleases, with naked DNA having a cytoplasmic half-life of <2 hr (Lechardeur et al. (1999) Gene Therapy, 6(4), p. 482). It was previously reported that transfection efficiency can increase as the total amount of DNA used is increased while the amount of gene delivery vector of interest is kept constant (Susa et al. (2008) Molecular Biology Reports, 35(3), pp. 313-319; Pradhan & Gadgil (2012) Cytotechnology, 64(6), pp. 613-622, the contents of each of which are incorporated herein by reference in their entireties). Without wishing to be bound by theory, this phenomenon indicates that excess DNA can reduce cytoplasmic degradation via a competitive inhibition mechanism.

Unfortunately, it remains technically challenging to drastically increase to total DNA amount used in transfection, in large part due to the toxicity arising from innate immune sensors of cytoplasmic dsDNA (Wu & Chen (2014) Immunology, 32(1), pp. 461-488, which is incorporated herein by reference in its entirety). The present disclosure demonstrates, among other things, simultaneously inhibiting DNA nucleases while keeping the total amount of transfected nucleic acids low, for example, by co-delivering an mRNA encoding DNA mimic polypeptide along with a payload oligonucleotide (e.g., a DNA payload oligonucleotide). mRNA delivery enables us to transiently create a cytoplasmic environment that contains orders of magnitude more DNA mimics than the actual DNA vector delivered. The present disclosure also encompasses the recognition that a strategy for reducing degradation of oligonucleotides may broadly target the common DNA-binding properties of various classes of nucleases. Supporting the viability of this approach, some bacteriophage have evolved DNA mimicking proteins which are able to overcome broad ranges of restriction enzyme systems.

In some embodiments, a helper polypeptide is or comprises a DNA mimic polypeptide. DNA mimic polypeptides are a class of polypeptides that structurally and electrostatically mimic dsDNA used by phage to competitively inhibit bacterial restriction systems (Wang et al. (2014) Biochemistry, 53(18), pp. 2865-2874, which is incorporated herein by reference in its entirety). In some embodiments, a DNA mimic polypeptide is selected from any one of Ocr polypeptide, antirestriction protein (ArdA), NuiA polypeptide, HI11450 polypeptide, DMP12 polypeptide, MfpA polypeptide, Arn polypeptide, Gam polypeptide and/or variants thereof. In some embodiments, a DNA mimic polypeptide (e.g., as described herein) is from a bacteriophage. In some embodiments, a helper polypeptide is a fully engineered DNA mimic. Engineered mimics can be designed using methods known in the art, for example, Yuksel et al. (2015) Mol. Biosyst., 12: 169-177, which is incorporated herein by reference in its entirety).

In some embodiments, a DNA mimic polypeptide is or comprises an Ocr polypeptide or a variant thereof.

In some embodiments, a DNA mimic polypeptide is or comprises an Arda polypeptide or a variant thereof.

In some embodiments, a DNA mimic polypeptide is or comprises a polypeptide derived or obtained from marine sediment metagenome (e.g., LCGC14_278712 as represented by Accession No. KKK84065 in GenBank or LCGC14_2905220 as represented by Accession No. KKK72305 in GenBank).

In some embodiments, a DNA mimic polypeptide is or comprises a polypeptide derived or obtained from a conjugative transposon protein, e.g., derived or obtained from human gut metagenome. An exemplary conjugative transposon protein includes, but is not limited to one represented by Accession No. EKC78327 in GenBank.

In some embodiments, a DNA mimic polypeptide is or comprises an antirestriction protein (ArdA) derived or obtained from human gut metagenome. An exemplary conjugative transposon protein includes, but is not limited to one represented by Accession No. EKC62359 in GenBank or by Accession No. EKC78842 in GenBank.

In some embodiments, delivery of a DNA mimic helper polypeptide reduces degradation of an oligonucleotide comprising a payload sequence. For example, in some embodiments, delivery of a DNA mimic helper polypeptide reduces degradation of an oligonucleotide comprising a payload sequence by at least about 30%, including, e.g., at least about 40%, at least about 50%, at least about 60%, at least about 70%, at least about 80%, at least about 90% or more, as compared to degradation of an oligonucleotide comprising a payload sequence introduced into a target cell in the absence of a DNA mimic helper polypeptide. In some embodiments, delivery of a DNA mimic helper polypeptide increases persistence of an oligonucleotide comprising a payload sequence. For example, in some embodiments, delivery of a DNA mimic helper polypeptide increases persistence of an oligonucleotide comprising a payload sequence in a target cell by at least about 30%, including, e.g., at least about 40%, at least about 50%, at least about 60%, at least about 70%, at least about 80%, at least about 90% or more, as compared to persistence of an oligonucleotide comprising a payload sequence introduced into a target cell in the absence of a DNA mimic helper polypeptide. In some embodiments, delivery of a DNA mimic helper polypeptide increases expression and/or activity of an oligonucleotide comprising a payload sequence in a target cell. For example, in some embodiments, delivery of a DNA mimic helper polypeptide increases expression and/or activity of an oligonucleotide comprising a payload sequence in a target cell by at least about 30%, including, e.g., at least about 40%, at least about 50%, at least about 60%, at least about 70%, at least about 80%, at least about 90% or more, as compared to expression and/or activity of an oligonucleotide comprising a payload sequence introduced into a target cell in the absence of a DNA mimic helper polypeptide.

Immunomodulatory Polypeptides

The present disclosure demonstrates that use of an immunomodulatory polypeptide as a helper polypeptide, e.g., an immunomodulatory polypeptide that suppresses or inhibits innate immunity pathways of host cells (“modulator of innate immunity”), can improve the effectiveness of payload oligonucleotides introduced in the host cells, e.g., by inhibiting host immunity-triggered suppression of protein translation and mRNA degradation, enhancing the expression and/or activity of a payload oligonucleotide in host cells, reducing non-specific toxicity in host cells by a payload oligonucleotide, and/or increasing viability of cells upon introduction of a payload oligonucleotide, e.g., by at least about 30%, at least about 40%, at least about 50%, at least about 60%, at least about 70%, at least about 80%, at least about 90% or more, as compared to that when a payload oligonucleotide is introduced into host cells in the absence of an immunomodulatory polypeptide. In some embodiments, delivery of an immunomodulatory polypeptide (e.g., a modulator of innate immunity) can increase persistence of an oligonucleotide comprising a payload sequence in host cells, e.g., by at least about 30%, at least about 40%, at least about 50%, at least about 60%, at least about 70%, at least about 80%, at least about 90% or more, as compared to that when a payload oligonucleotide is introduced into host cells in the absence of an immunomodulatory polypeptide. In some embodiments, delivery of an immunomodulatory polypeptide (e.g., a modulator of innate immunity) can increase expression and/or activity of an oligonucleotide comprising a payload sequence in host cells, e.g., by at least about 30%, at least about 40%, at least about 50%, at least about 60%, at least about 70%, at least about 80%, at least about 90% or more, as compared to that when a payload oligonucleotide is introduced into host cells in the absence of an immunomodulatory polypeptide.

Non-immune somatic cells detect the presence of foreign RNA (e.g., mRNA) using sensor proteins, including, e.g., but not limited to retinoic acid inducible gene I (RIG-1), melanoma differentiation-associated antigen 5 (MDA5), protein kinase R (PKR) and 2′-5′-oligoadenylate synthetase (OAS) (Sahin et al. (2014) Nature Reviews Drug Discovery 13: 759-780, which is incorporated by reference in its entirety). Innate immune activation by RIG-I, which senses 5′ triphosphates characteristic of uncapped viral transcripts, and MDA5, which detects long dsRNA, can be ameliorated, e.g., via using non-standard base chemistries to make mRNA therapeutics (Karikó et al. (2011) Immunity 23: 165-175; Mu et al. (2018) Nucleic Acids Research 46(10):5239-5249, each of which is incorporated herein by reference in its entirety). Short stretches of dsRNA, sensed by PKR and the OAS proteins, are more difficult to evade host innate immunity than long dsRNA due to the presence of structured mRNA in many naturally occurring human transcripts (Mortimer et al. (2014) Nat Rev Genet. 15:469-79, which is incorporated herein by reference in its entirety).

Cytoplasmic nucleic acids can act as ligands for pro-inflammatory pattern recognition receptors (Wu and Chen (2014) Immunology, 32: 461-488, which is incorporated herein by reference in its entirety). For example, antigen-presenting cells express TLR9, which recognizes unmethylated CpG dsDNA, and TLR3, which is triggered by dsRNA. RIG-I and MAVS detect dsRNA and, albeit to a lesser extent, DNA in nearly all mammalian cell types (Cheng et al. (2007) Proc. National Acad. Sci., 104: 9035-9040, which is incorporated by reference in its entirety). Detection of DNA is primarily mediated by the cGAS-STING pathway (Li et al. (2013) Immunity, 39: 1019-1031). Certain DNA viruses have evolved mechanisms to regulate and circumvent cGAS-STING and other pathways that regulate innate immunity. The present disclosure encompasses the recognition that expression of immunomodulatory polypeptides, e.g., ones that inhibit innate immunity pathway component, can increase persistence and/or reduce degradation of oligonucleotides for delivery.

Accordingly, in some embodiments, a helper polypeptide in any aspects described herein is or comprises an immunomodulatory polypeptide.

In some embodiments, an immunomodulatory polypeptide is or comprises a modulator of innate immunity. In some embodiments, a modulator of innate immunity inhibits or suppresses a cGAS-STING pathway. In some embodiments, a modulator of innate immunity that inhibits or suppresses a cGAS-STING is a viral polypeptide. Examples of viral regulators of the cGAS-STING pathway are known in the art, e.g., as described in Table 1 of Ma and Damania (2016) Cell Host & Microbe 19: 150-158 (Review), which is incorporated herein by reference in its entirety), and can be used as helper polypeptides in any aspects described herein. In some embodiments, a viral modulator of innate immunity includes, for example, but is not limited to a viral interferon regulatory factor (vIRF1) polypeptide, a ORF52 polypeptide (e.g., Kaposi's sarcoma-associated herpesvirus (KSHV) ORF52 (also known as KSHV inhibitor of cGAS [KicGAS]), a PLP2-TM polypeptide (e.g., a membrane anchored papain-like protease (PLP) domain, e.g., from human coronavirus (HCoV) NL63), a PLP2 polypeptide, and/or variants thereof.

In some embodiments, an immunomodulatory polypeptide reduces expression and/or activity of at least one or more of RIG-I, MDA5, PKR, and OAS. In some embodiments, an immunomodulatory polypeptide is an inhibitor of RIG-I. In some embodiments, an immunomodulatory polypeptide is an inhibitor of MDA5. In some embodiments, an immunomodulatory polypeptide is an inhibitor of PKR. In some embodiments, an immunomodulatory polypeptide is an inhibitor of OAS. In some embodiments, an immunomodulatory polypeptide is an inhibitor of RIG-I and MDA5. In some embodiments, an immunomodulatory polypeptide is an inhibitor of PKR and OAS. In some embodiments, an immunomodulatory polypeptide is an inhibitor of RIG-I, MDA5, PKR, and OAS.

In some embodiments, an immunomodulatory polypeptide that reduces expression and/or activity of at least one or more of RIG-I, MDA5, PKR, and OAS employs a viral mechanism to evade host innate immunity. In some embodiments, such an immunomodulatory polypeptide is a viral polypeptide, e.g., a polypeptide obtained or derived from a virus, that suppresses or inhibits host innate immunity pathway, e.g., associated with RIG-I, MDA5, PKR, and/or OAS pathways.

Viral immunomodulatory polypeptides of any types described herein can be obtained or derived from dsRNA viruses (e.g., Adenoviruses, Herpesviruses, Poxviruses), ssDNA viruses (e.g., Parvoviruses), dsRNA viruses (e.g., Reoviruses), (+)ssRNA viruses (single-stranded positive-sense RNA viruses, e.g., Picornaviruses, Togaviruses), (−)ssRNA viruses (single-stranded negative-antisense RNA viruses, e.g., Orthomyxoviruses, Rhabdoviruses), ssRNA-RT viruses (single-stranded positive-sense RNA viruses with reverse transcriptase (RT) and/or DNA intermediates in life-cycle (e.g., Retroviruses), dsDNA-RT viruses (double-stranded reverse transcribing viruses with RNA intermediates in life-cycle, e.g., Hepadnaviruses.

In some embodiments, an immunomodulatory polypeptide is a polypeptide derived or obtained from dsRNA viruses. For example, in some embodiments, an immunomodulatory polypeptide is or includes a herpesvirus polypeptide, e.g., a herpes simplex virus (HSV) polypeptide. In some embodiments, a viral immunomodulatory polypeptide is or includes a herpes simplex virus type 1 (HSV-1) polypeptide, e.g., a HSV-1 tegument polypeptide.

In some embodiments, an immunomodulatory polypeptide is or includes an RNA-binding domain of a US11 polypeptide. In some embodiments, an immunomodulatory polypeptide is or includes a US11 polypeptide. In some embodiments, a US11 polypeptide can inhibit at least one (including, e.g., one, two, three, or four) of RIG-I, MDA5, PKR, and OAS RNA sensors present in non-immune cells. In some embodiments, a US11 polypeptide (e.g., including an RNA-binding domain of a US11 polypeptide) can bind to and block the phosphorylation of PKR (Cassady & Gross (2002) Journal of Virology 76:2029-35, which is incorporated by reference in its entirety), directly interact with and inhibits MDA5 and RIG-I (Xing et al. (2012) Journal of Virology 86: 3528-3540, which is incorporated by reference in its entirety), and/or block OAS-dsRNA binding (Sanchez & Mohr (2007) Journal of Virology 81: 3455-3464, which is incorporated by reference in its entirety). In some embodiments, a US11 polypeptide (e.g., including an RNA-binding domain of a US11 polypeptide) can inhibit PKR and/or OAS in mitochondrial antiviral signaling (MAVS) knock-out (KO) cells. In some embodiments, a US11 polypeptide (e.g., including an RNA-binding domain of a US11 polypeptide) can inhibit PKR-driven protein degradation and/or OAS-drive RNAse activity.

In some embodiments, a US11 polypeptide (e.g., including an RNA-binding domain of a US11 polypeptide) includes an amino acid sequence that is based on the corresponding domain(s) of tegument US11 polypeptide from HSV-1. For example, a US11 polypeptide (alternatively called γ134.5) is encoded in two copies by the herpes simplex virus type 1 (HSV-1) genome, and has a uniquely broad role in the suppression of innate immunity (Chou et al. (1990) Science 250: 1262-6, which is incorporated by reference in its entirety). This immune suppression function is desirable in HSV-1 because despite being a dsDNA virus, more than half of the HSV-1 genome forms dsRNA side-products (Jacquemont & Roizman (1975) Journal of Virology 15: 707-13, which is incorporated by reference in its entirety).

In some embodiments, a US11 polypeptide (e.g., including an RNA-binding domain of a US11 polypeptide) can be a US11 homologue from other herpes viruses or viral families, which may have acquired US11-type proteins via horizontal gene transfer.

In some embodiments, a US11 polypeptide comprises the sequence of SEQ ID NO: 1, which is set forth below:

(SEQ ID NO: 1)
MSQTQPPAPVGPGDPDVYLKGVPSAGMHPRGVHAPRGHPRMISGPPQRGD
NDQAAGQCGDSGLLRVGADTTISKPSEAVRPPTIPRTPRVPREPRVPRPP
REPREPRVPRAPRDPRVPRDPRDPRQPRSPREPRSPREPRSPREPRTPRT
PREPRTARGSV

In some embodiments, a US11 polypeptide comprises the sequence of SEQ ID NO: 2, which is set forth below:

(SEQ ID NO: 2)
MPRVPRPPREPREPRVPRAPRDPRVPRDPRDPRQPRSPREPRSPREPRSP
REPRTPRTPREPRTARGSV.

In some embodiments, immunomodulatory polypeptides described herein are delivered via oligonucleotides. In some embodiments, immunomodulatory polypeptides described herein are delivered via RNA oligonucleotides. In some embodiments, an RNA oligonucleotide that encodes an immunomodulatory polypeptide (e.g., ones described herein) is a mRNA oligonucleotide. Delivering immunomodulatory polypeptides (e.g., ones described herein) to target cells via mRNA oligonucleotides may be more advantageous in certain aspects than protein-based delivery. For example, some proteins cannot traverse the cellular membrane due to their large size. Additionally, in the context of delivery of RNA oligonucleotides, immunomodulatory polypeptides (e.g., ones described herein) delivered via mRNA oligonucleotides can have an advantage of matching expression kinetics and cellular localization of payload mRNA oligonucleotides.

Synthetic Cell Surface Receptor Polypeptides

Viruses frequently regulate host membrane receptors in order to facilitate uptake and release of viral particles. The present disclosure encompasses the recognition that expression of one or more synthetic receptor polypeptides may facilitate uptake of an oligonucleotide for delivery by a cell to which the oligonucleotide is delivered.

In some embodiments, a helper polypeptide is or comprises a synthetic cell surface receptor polypeptide. Synthetic cell surface receptor polypeptides include, for example, TVA-EGF polypeptide, H-EGF polypeptide, H-IGF1 polypeptide and/or variants thereof.

In some embodiments, delivery of a helper polypeptide that is a synthetic cell surface receptor polypeptide increases uptake of an oligonucleotide comprising a payload sequence by a target cell, e.g., by at least about 30%, at least about 40%, at least 50%, at least about 60%, at least about 70%, at least about 80%, at least about 90%, or more, as compared to uptake of an oligonucleotide comprising a payload sequence by a target cell in the absence of a helper polypeptide. In some embodiments, delivery of a helper polypeptide that is a synthetic cell surface receptor polypeptide increases availability of an oligonucleotide comprising a payload sequence in a target cell, e.g., by at least about 30%, at least about 40%, at least 50%, at least about 60%, at least about 70%, at least about 80%, at least about 90%, or more, as compared to availability of an oligonucleotide comprising a payload sequence in a target cell in the absence of a helper polypeptide. In some embodiments, delivery of a helper protein that is a synthetic cell surface receptor polypeptide increases expression and/or activity of an oligonucleotide comprising a payload sequence, e.g., by at least about 30%, at least about 40%, at least 50%, at least about 60%, at least about 70%, at least about 80%, at least about 90%, or more, as compared to expression and/or activity of an oligonucleotide comprising a payload sequence in a target cell in the absence of a helper polypeptide.

Payload Oligonucleotides

Payload oligonucleotides are oligonucleotides each comprising a payload sequence. Payload sequences are generally any sequence of interest (e.g., a sequence that encodes a target payload such as a target peptide or polypeptide) that are desired to be introduced into a cell, tissue, organ, organism, and/or system comprising cells. In some embodiments, a payload sequence encodes a single target peptide or polypeptide. In some embodiments, a payload sequence encodes a fusion polypeptide and/or a chimeric polypeptide, e.g., a payload sequence encoding at least two or more peptides or polypeptides. In some embodiments, a payload sequence comprises a synthetic nucleic acid.

In some embodiments, an oligonucleotide comprising a payload sequence is part of a non-viral vector. In some embodiments, an oligonucleotide comprising a payload sequence is a DNA oligonucleotide. In some embodiments, an oligonucleotide comprising a payload sequence is an RNA oligonucleotide (e.g., a mRNA).

In some embodiments, an oligonucleotide comprising a payload sequence is a non-viral DNA oligonucleotide.

In some embodiments, an oligonucleotide comprising a payload sequence is an RNA oligonucleotide. In some embodiments, an oligonucleotide comprising a payload sequence is an mRNA oligonucleotide. In some embodiments, an mRNA oligonucleotide comprises a target payload-encoding open reading frame (ORF), a poly(A) tail at the 3′ end, and a “cap,” e.g., a 7-methyl-guanosine residue joined to the 5′-end via a 5′-5′ triphosphate.

In some embodiments of any aspects described, oligonucleotides comprising a payload sequence include an extension sequence at their 5′ and/or 3′ ends. In some embodiments, oligonucleotides comprising a payload sequence further comprise an additional element, including, but not limited to, spacers, recombination elements, binding motifs, etc.

In some embodiments, an oligonucleotide comprising a payload sequence comprises one or more of: an encoding region, a gene regulatory element, and a transcription terminator. Non-limiting examples of gene regulatory elements include promoters, transcriptional activators, enhancers, and polyadenylation signals. In some embodiments, the payload sequence comprises an encoding region, a gene regulatory element, and a transcription terminator, positioned relative to each other such that the encoding region is between the gene regulatory element and the transcription terminator.

In some embodiments, an encoding region encodes a gene product. In some embodiments, the gene product is an RNA. In some embodiments, an encoding region encodes a polypeptide (such as a protein, such as a glycoprotein). In some embodiments, an encoding region encodes a fusion polypeptide and/or a chimeric polypeptide. In some embodiments, the encoding region encodes one gene product. In some embodiments, the encoding region encodes more than one gene product (e.g., 2, 3, 4, 5, 6, 7 or more gene products). In some embodiments, an encoding region encodes a regulatory RNA (e.g., a siRNA, microRNA, etc.).

In some embodiments, a payload sequence comprises one or more aptamer- or polypeptide-binding domains (e.g., transcription factor binding domains).

In some embodiments, a payload sequence comprises a synthetic nucleic acid.

In some embodiments, an oligonucleotide comprising a payload sequence contains a region of homology to a sequence in the genome of a target cell (a “homology arm”). In some embodiments, an oligonucleotide comprising a payload sequence comprises homology arms (e.g., two homology arms). In some embodiments, a homology arm is or comprises a sequence that is homologous to a target site and/or a region flanking a target site in the genome of a target cell.

Without wishing to be bound by theory, it is envisioned that in some embodiments, homology arms can be used as a template for homologous recombination. In some embodiments, a payload sequence from an oligonucleotide that includes one or more homology arms can be inserted into the genome of a target cell via homologous recombination. In some embodiments, this homologous recombination event utilizes the endogenous cell machinery. In some embodiments, this homologous recombination event utilizes an exogenously co-expressed targeted nuclease.

In some embodiments, a homology arm is 50 bp to 10,000 bp in length. In some embodiments, a homology are is about 50 bp, 100 bp, 200 bp, 300 bp, 400 bp, 500 bp, 1000 bp, 1500 bp, 2000 bp, 2500 bp, 3000 bp, 4000 bp, 5000 bp, 6000 bp, 8000 bp, 10,000 bp or any value therebetween.

In some embodiments, homologous recombination is used to integrate a payload sequence into the genome of a cell.

In some embodiments, an oligonucleotide comprising a payload sequence associates with a helper polypeptide.

In some embodiments, oligonucleotide comprising a payload sequence further comprises a nuclear import sequence. In some embodiments, a nuclear import sequence is a synthetic nuclear import sequence with a repeated transcription factor binding domain motif. In some embodiments, a synthetic nuclear import sequence comprises two or more polypeptide binding motifs and one or more spacer sequences, wherein the two or more polypeptide binding motifs include a first polypeptide binding motif and a second polypeptide binding motif, wherein the first polypeptide binding motif is a reverse complement of the second polypeptide binding motif, and wherein each spacer sequence is flanked by two polypeptide binding motifs. In some embodiments, the first and second polypeptide binding motifs are orthogonal (with respect to species origin) to the payload sequence, e.g., the binding motif(s) are from one species and the payload sequence is from another.

A payload sequence can be of any length, for example, between 2 and 100,000,000 nucleotides in length (or any integer value therebetween). In some embodiments, a payload sequence comprises at least 20 nucleotides, at least 50 nucleotides, at least 75 nucleotides, at least 100 nucleotides, at least 150 nucleotides, at least 200 nucleotides, at least 250 nucleotides, at least 300 nucleotides, at least 350 nucleotides, at least 400 nucleotides, at least 450 nucleotides, at least 500 nucleotides, at least 550 nucleotides, at least 600 nucleotides, at least 650 nucleotides, at least 700 nucleotides, at least 750 nucleotides, at least 800 nucleotides, at least 850 nucleotides, at least 900 nucleotides, at least 950 nucleotides, at least 1000 nucleotides, at least 1100 nucleotides, at least 1200 nucleotides, at least 1300 nucleotides, at least 1400 nucleotides, at least 1500 nucleotides, at least 1600 nucleotides, at least 1700 nucleotides, at least 1800 nucleotides, at least 2000 nucleotides, at least 2500 nucleotides, at least 3000 nucleotides, at least 3000 nucleotides, at least 4000 nucleotides, at least 5000 nucleotides, at least 6000 nucleotides, at least 7000 nucleotides, at least 8000 nucleotides, at least 9000 nucleotides, at least 10,000 nucleotides, at least 11,000 nucleotides, at least 12,000 nucleotides, at least 13,000 nucleotides, at least 14,000 nucleotides, at least 15,000 nucleotides, at least 16,000 nucleotides, at least 17,000 nucleotides, at least 18,000 nucleotides, at least 19,000 nucleotides, at least 20,000 nucleotides, at least 21,000 nucleotides, at least 22,000 nucleotides, at least 23,000 nucleotides, at least 24,000 nucleotides, or at least 25,000 nucleotides.

In some embodiments, an oligonucleotide comprising a payload sequence is between 50 and 25,000 nucleotides in length, between 100 and 20,000 nucleotides in length, between 500 and 10,000 nucleotides in length, between 1,000 and 8,000 nucleotides in length, and/or between 2,000 and 5,000 nucleotides in length.

In some embodiments, payload polypeptides described herein are delivered as RNA oligonucleotides. In some embodiments, payload polypeptides described herein are mRNA oligonucleotides. In some embodiments, an RNA oligonucleotide (e.g., an mRNA oligonucleotide) comprising a payload sequence is between 50 and 25,000 nucleotides in length, between 100 and 20,000 nucleotides in length, between 500 and 10,000 nucleotides in length, between 250 and 2000 nucleotides in length, between 500 and 1500 nucleotides in length, between 1,000 and 8,000 nucleotides in length, and/or between 2,000 and 5,000 nucleotides in length.

In some embodiments, payload polypeptides described herein are delivered as DNA oligonucleotides. In some embodiments, a DNA oligonucleotide comprising a payload sequence is between 50 and 25,000 nucleotides in length, between 100 and 20,000 nucleotides in length, between 500 and 10,000 nucleotides in length, between 250 and 2000 nucleotides in length, between 500 and 1500 nucleotides in length, between 1,000 and 8,000 nucleotides in length, and/or between 2,000 and 5,000 nucleotides in length.

Targeted Nucleases

In some embodiments, expression systems and methods of the present disclosure include targeted nucleases. In some embodiments, a targeted nuclease directs insertion of a payload sequence into the genome of a cell.

In some embodiments, one or more targeted nucleases as described herein can create a double-stranded break in a target sequence (e.g., cellular chromatin) at a predetermined site, and a payload oligonucleotide that includes sequences with homology to the nucleotide sequence in the region of the break, can be introduced into the cell. Targeted nuclease-mediated genome cleavage at a desired location can be obtained by the use of an engineered nuclease. For example, a double-strand break (DSB) for can be created by a targeted nuclease such as a zinc-finger nuclease (ZFN) or TAL effector domain nuclease (TALEN).

Another nuclease system involves the use of a so-called acquired immunity system found in bacteria and archaea known as CRISPR/Cas. CRISPR/Cas systems are found in 40% of bacteria and 90% of archaea and differ in the complexities of their systems. See, e.g., U.S. Pat. No. 8,697,359. CRISPR loci (clustered regularly interspaced short palindromic repeat) is a region within the organism's genome where short segments of foreign DNA are integrated between short repeat palindromic sequences. These loci are transcribed and the RNA transcripts (“pre-crRNA”) are processed into short CRISPR RNAs (crRNAs). There are three types of CRISPR/Cas systems which all incorporate these RNAs and proteins known as “Cas” proteins (CRISPR associated). Types I and III both have Cas endonucleases that process the pre-crRNAs, that, when fully processed into crRNAs, assemble a multi-Cas protein complex that is capable of cleaving nucleic acids that are complementary to the crRNA.

In type II systems, crRNAs are produced using a different mechanism where a trans-activating RNA (tracrRNA) complementary to repeat sequences in the pre-crRNA, triggers processing by a double strand-specific RNase III in the presence of the Cas9 protein. Cas9 is then able to cleave a target DNA that is complementary to the mature crRNA however cleavage by Cas 9 is dependent both upon base-pairing between the crRNA and the target DNA, and on the presence of a short motif in the crRNA referred to as the PAM sequence (protospacer adjacent motif) (see, e.g., Qi et al (2013) Cell 152: 1173). In addition, a tracrRNA may be required in some systems, as it base pairs with the crRNA at its 3′ end, and this association triggers Cas9 activity.

A Cas9 protein has at least two nuclease domains: one nuclease domain is similar to a HNH endonuclease, while the other resembles a Ruv endonuclease domain. HNH-type domains appear to be responsible for cleaving the DNA strand that is complementary to the crRNA while the Ruv domain cleaves the non-complementary strand.

Use of a crRNA-tracrRNA complex can be avoided, for example, by use of an engineered “single-guide RNA” (sgRNA) that comprises the hairpin normally formed by the annealing of the crRNA and the tracrRNA (see, e.g., Jinek et al (2012) Science 337:816).

In some embodiments, a targeted nuclease is a zinc-finger nuclease (ZFN), TAL effector domain nuclease (TALEN), or an engineered CRISPR/Cas9 system.

In some embodiments, a payload sequence may be physically integrated into the genome or, alternatively, a payload oligonucleotide may be used as a template for repair of the break via homologous recombination, resulting in the introduction of all or part of the nucleotide sequence into the genome. In some certain embodiments, homologous recombination is used to integrate a payload sequence into the genome of a cell.

In some embodiments, a targeted nuclease may further comprise at least one of a nuclear localization signal (NLS) polypeptide, a nuclear export signal (NES), or a functional domain. In some embodiments, a NLS polypeptide, NES and/or functional domain may be conditionally activated or inactivated.

In some embodiments, co-expression of a targeted nuclease increases the number of recombination events that in a target cell or cells. In some embodiments, co-expression of a targeted nuclease increases the number of recombination events 2 fold, 5 fold, 10 fold, 50 fold, 100 fold, 500 fold, 1000 fold or more.

Homologous recombination is a multi-step process involving modification of DNA ends and recruitment of certain cellular factors into a protein complex. In some embodiments, expression systems can include one or more additional exogenous factors, along with an oligonucleotide comprising a payload sequence and a targeted nuclease, to facilitate homologous recombination.

### II. Vectors

The present disclosure provides vectors for delivery of oligonucleotide sequences in the context of the present disclosure. In some embodiments, a vector comprises an oligonucleotide comprising a payload sequence. In some embodiments, a vector comprises an oligonucleotide comprising a sequence that encodes a helper polypeptide. In some embodiments, a vector comprises an oligonucleotide encoding a targeted nuclease. In some embodiments, a vector comprises two or more of an oligonucleotide comprising a payload sequence, an oligonucleotide comprising a sequence that encodes a helper polypeptide and an oligonucleotide encoding a targeted nuclease.

Generally, vectors in the context of the present disclosure are capable of transferring or delivering oligonucleotide sequences to target cells. In some embodiments, a vector is a cloning vector. In some embodiments, a vector is an expression vector. In some embodiments, a vector is an integrating vector. In some embodiments, a vector is a non-viral vector.

In some embodiments, a vector is a DNA vector. Any suitable DNA vector known in the art can be used in the context of the present disclosure. In some embodiments, a vector is a non-viral DNA vector. In some embodiments, a DNA vector comprises an oligonucleotide comprising a payload sequence. In some embodiments, a DNA vector comprises an oligonucleotide comprising a sequence that encodes a helper polypeptide.

In some embodiments, a vector is an RNA vector. Any suitable RNA vector known in the art can be used in the context of the present disclosure. In some embodiments, a RNA vector comprises an oligonucleotide comprising a payload sequence. In some embodiments, a RNA vector comprises an oligonucleotide comprising a sequence that encodes a helper polypeptide.

In some embodiments, a vector includes both an oligonucleotide comprising a payload sequence and an oligonucleotide comprising a sequence that encodes a helper polypeptide.

In some particular embodiments, a vector including a oligonucleotide comprising a payload sequence is a non-viral DNA vector and a vector including an oligonucleotide comprising a sequence that encodes a helper polypeptide is an RNA vector.

In some embodiments, vectors in the context of the present disclosure are plasmids. In some embodiments, a plasmid comprises one or more of: an oligonucleotide comprising a payload sequence, an oligonucleotide comprising a sequence that encodes a helper polypeptide and an oligonucleotide encoding a targeted nuclease.

In some embodiments, a vector is a linearized vector.

In some embodiments, an oligonucleotide comprising a payload sequence and/or an oligonucleotide comprising a sequence that encodes a helper polypeptide is a linear covalently closed (lcc) nucleic acid vector. In some embodiments, lcc vectors are DNA vectors. In some embodiments, lcc vectors are RNA vectors (e.g., an mRNA vector).

In some embodiments, provided lcc vectors are a single strand of a nucleic acid including a first payload sequence, a second payload sequence hybridized to the first payload sequence, and first and second end regions as described herein, with the 5′ end of the first end region being covalently bound to the 3′ end of the first payload sequence, the 3′ end of the first end region being covalently bound to the 5′ end of the second payload sequence, the 5′ end of the second end sequence being covalently bound to the 3′ end of the second payload sequence, and the 3′ end of the second end sequence being covalently bound to the 5′ end of the first payload sequence.

In some embodiments, there are at least 30 nucleotides between each end (as described herein) of the lcc vector and the closest 5′ nucleotide of the first or second payload sequences to the respective end. In some embodiments, there are at least 40, 45, 50, 55, 60, 65, 70, 75, 80, 85, 90, 95, 100, 105, 110, 115, or 120 nucleotides between each end and the closest 5′ nucleotide of the first or second payload sequences to the respective end.

In some embodiments in which the payload sequence comprises an encoding region, a gene regulatory element, and a transcription terminator, positioned relative to each other such that the encoding region is between the gene regulatory element and the transcription terminator, there are at least 30 nucleotides between the gene regulatory element and the end proximal to the gene regulatory element, and there are at least 30 nucleotides between the transcription terminator and the end proximal to the transcription terminator. In some embodiments, there are at least 40, 45, 50, 55, 60, 65, 70, 75, 80, 85, 90, 95, 100, 105, 110, 115, or 120 nucleotides between the gene regulatory element and the end proximal to the gene regulatory element. In some embodiments, there are at least 40, 45, 50, 55, 60, 65, 70, 75, 80, 85, 90, 95, 100, 105, 110, 115, or 120 nucleotides between the transcription terminator and the end proximal to the transcription terminator.

### III. Compositions

The present disclosure provides compositions comprising any component, or combination of components, of a nucleic acid expression system as described herein. In some embodiments, the compositions described herein are useful for improving the delivery of oligonucleotides (e.g., RNA or DNA oligonucleotides) comprising a payload sequence. In some embodiments, the compositions described herein are useful for improving the effectiveness of RNA-based therapeutics and vaccines. In some embodiments, the compositions described herein are useful for reducing non-specific toxicity induced by oligonucleotide-based therapeutics and vaccines. In some embodiments, the compositions described herein are useful for reducing innate immunity-triggered suppression of protein translation and/or mRNA degradation. In some embodiments, the compositions described herein are useful for enhancing expression and/or activity of a payload sequence to be introduced into target cells.

In some embodiments, a composition comprises at least one oligonucleotide comprising a payload sequence as described herein. In some embodiments a composition comprises 1, 2, 3, 4, 5, 6, 7, 8, 9, or 10 oligonucleotides, each comprising a payload sequence.

In some embodiments, a composition comprises least one oligonucleotide comprising a sequence that encodes a helper polypeptide as described herein. In some embodiments a composition comprises 1, 2, 3, 4, 5, 6, 7, 8, 9, or 10 oligonucleotides, each encoding a unique helper polypeptide. In some embodiments, a composition comprises at least one oligonucleotide comprising a sequence that encodes a helper polypeptide, wherein a helper polypeptide is or comprises a nuclear localization signal (NLS) polypeptide, a DNA mimic polypeptide, an immunomodulatory polypeptide (e.g., a modulator of innate immunity), or a synthetic cell surface receptor polypeptide. In some embodiments, a composition comprises at least 2, 3, 4, 5, 6, 7, 8, 9, or 10 oligonucleotides, each comprising a sequence that encodes a helper polypeptide, wherein each helper polypeptide independently is or comprises a nuclear localization signal (NLS) polypeptide, a DNA mimic polypeptide, an immunomodulatory polypeptide (e.g., a modulator of innate immunity), or a synthetic cell surface receptor polypeptide. In some embodiments involving any compositions described herein, a helper polypeptide is or comprises a DNA mimic polypeptide (e.g., ones described herein). In some embodiments involving any compositions described herein, a helper polypeptide is or comprises an immunomodulatory polypeptide (e.g., ones described herein). In some embodiments, a composition comprises or further comprises least one (e.g., 1, 2, 3, 4, 5, 6, 7, 8, 9, or 10) RNA oligonucleotide comprising a sequence that encodes a US11 polypeptide (e.g., one as described herein).

In some embodiments, a composition comprises at least one oligonucleotide comprising a payload sequence and at least one oligonucleotide comprising a sequence that encodes a helper polypeptide. In some embodiments, a composition comprises at least one oligonucleotide comprising a payload sequence and at least 2, 3, 4, 5, 6, 7, 8, 9, or 10 oligonucleotides, each comprising a sequence that encodes a helper polypeptide. In some embodiments involving any compositions described herein, a helper polypeptide is or comprises a DNA mimic polypeptide (e.g., ones described herein). In some embodiments involving any compositions described herein, a helper polypeptide is or comprises an immunomodulatory polypeptide (e.g., ones described herein). In some embodiments, a composition comprises or further comprises least one (e.g., 1, 2, 3, 4, 5, 6, 7, 8, 9, or 10) RNA oligonucleotide comprising a sequence that encodes a US11 polypeptide (e.g., one as described herein).

In some embodiments, a composition comprises any embodiment of a nucleic acid expression system described herein.

In some embodiments involving any compositions described herein, an oligonucleotide comprising a payload sequence is a DNA oligonucleotide and an oligonucleotide comprising a sequence that encodes a helper polypeptide (e.g., ones described herein) is a DNA oligonucleotide. In some embodiments involving any compositions described herein, an oligonucleotide comprising a payload sequence is a DNA oligonucleotide and an oligonucleotide comprising a sequence that encodes a helper polypeptide (e.g., ones described herein) is an RNA (e.g., mRNA) oligonucleotide. In some embodiments involving any compositions described herein, an oligonucleotide comprising a payload sequence is an RNA (e.g., mRNA) oligonucleotide and an oligonucleotide comprising a sequence that encodes a helper polypeptide (e.g., ones described herein) is an RNA (e.g., mRNA) oligonucleotide.

In some embodiments where oligonucleotides are RNA oligonucleotides, RNA oligonucleotides (e.g., comprising a payload sequence or encoding a US11 polypeptide) in any of nucleic acid expression systems and/or compositions described herein may be delivered as naked RNA oligonucleotides or complexed with a complexing agent, e.g., for protecting RNA oligonucleotides from degradation, and/or for facilitating cell delivery. Exemplary complexing agents include, but are not limited to lipids, polymers, or small arginine-rich peptide such as protamine. In some embodiments, RNA oligonucleotides (e.g., comprising a payload sequence or encoding a US11 polypeptide) in any of nucleic acid expression systems and/or compositions described herein may be encapsulated, e.g., in liposomes or other suitable carriers.

In some embodiments, any of compositions described herein can be a pharmaceutical composition.

Compositions that Deliver Helper Polypeptides

In accordance with the present disclosure, any of a variety of modalities may be utilized to deliver one or more helper polypeptides described herein. To give but a few examples, in some embodiments, one or more helper polypeptides as described herein are administered (i.e., to a subject or system). In some embodiments, one or more oligonucleotide that each encodes a helper polypeptide may be administered; in some such embodiments, the one or more encoding oligonucleotides may each be associated with one or more elements that directs its expression. In some embodiments, a cell containing and/or expressing one or more helper polypeptides and/or oligonucleotides that encode the one or more helper polypeptides is administered. In some embodiments, a viral particle containing one or more helper polypeptides and/or oligonucleotides that encode the one or more helper polypeptides and/or expresses the one or more helper polypeptides is administered.

Thus, in some embodiments, one or more helper polypeptides described herein can be directly administered. As such, in some embodiments, a composition that delivers one or more helper polypeptides described herein includes one or more helper polypeptides described herein.

In some embodiments, one or more helper polypeptide described herein can be delivered by delivering an oligonucleotide that encodes one or more helper polypeptides described herein, a vector that includes such an oligonucleotide, a cell that includes an oligonucleotide that encodes one or more helper polypeptides described herein, a cell that includes a vector comprising one or more oligonucleotides that each encodes one or more helper polypeptides described herein, and/or a cell that includes one or more helper polypeptides described herein. As such, in some embodiments, a composition that delivers one or more helper polypeptides described herein includes one or more oligonucleotides that each encode one or more helper polypeptides described herein, a vector that includes one or more such oligonucleotides, a cell that includes one or more oligonucleotides that each encode one or more helper polypeptides described herein, a cell that includes a vector comprising one or more oligonucleotides that each encode one or more helper polypeptides described herein, and/or a cell that includes one or more helper polypeptides described herein.

In some embodiments, a helper polypeptide described herein can be delivered by delivering one or more viral particles that each comprise one or more oligonucleotides that each encode one or more helper polypeptides described herein, a vector that includes one or more oligonucleotides that each encode one or more helper polypeptides described herein, and/or one or more helper polypeptides described herein. As such, in some embodiments, a composition that delivers one or more helper polypeptides described herein includes one or more viral particles that each comprise one or more oligonucleotides that each encode one or more helper polypeptides described herein, a vector that includes one or more oligonucleotides that each encode one or more helper polypeptides described herein, and/or one or more helper polypeptides described herein. Exemplary oligonucleotides, vectors, cells and viral particles are described herein.

In some embodiments, a composition that delivers one or more helper polypeptides described herein can be a pharmaceutical composition.

Pharmaceutical Compositions

The present disclosure further provides pharmaceutical compositions comprising at least one oligonucleotide comprising a payload sequence and/or at least one oligonucleotide comprising a sequence that encodes a helper polypeptide as described herein and a pharmaceutically acceptable carrier or excipient.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

Formulations of the pharmaceutical compositions described herein may be prepared by any method known or hereafter developed in the art of pharmacology. In general, such preparatory methods include the step of bringing the active ingredient into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one oligonucleotide comprising a payload sequence and/or at least one oligonucleotide comprising a sequence that encodes a helper polypeptide as described herein.

Relative amounts of an oligonucleotide comprising a payload sequence and/or at least one oligonucleotide comprising a sequence that encodes a helper polypeptide as described herein, the pharmaceutically acceptable excipient, and/or any additional ingredients in a pharmaceutical composition in accordance with the disclosure can vary, depending upon the subject to be treated, target cells, and may also further depend upon the route by which the composition is to be administered.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Kits

The present disclosure further provides a pharmaceutical pack or kit comprising one or more containers filled with at least one oligonucleotide comprising a payload sequence and/or at least one oligonucleotide comprising a sequence that encodes a helper polypeptide as described herein. Kits may be used in any applicable method, including, for example, therapeutically or diagnostically. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Delivery

Provided compositions can be delivered to cells by any of a variety of known methods in the art, including, but not limited to, transfection into cells (e.g., via electroporation, chemical methods, etc.), delivery via particles (e.g., nanoparticles), and/or administration to an organism (e.g., by any suitable administration route). Cells to which vectors can be delivered can be, for example, cultured cells, and/or cells within a tissue and/or an organism.

In some embodiments, delivery is to a target cell. In some embodiments, a target cell is a mitotic cell. In some embodiments, a target cell is a meiotic cell. In some embodiments, a cell is a non-mitotic cell. In some embodiments, a cell is a non-dividing cell.

In some embodiments, a nucleic acid expression system in the context of the present disclosure can be used for oligonucleotide delivery to a target cell ex vivo. In some embodiments, an ex vivo cell can be in cell culture. In some embodiments, a nucleic acid expression system in the context of the present disclosure can be used for oligonucleotide delivery to a target cell in vivo. In some embodiments, a cell in vivo can be in a subject.

In some embodiments, components of a nucleic acid expression system described herein (e.g., an oligonucleotide comprising a payload and an oligonucleotide comprising a sequence that encodes a helper polypeptide) are delivered to a target cell. In some embodiments, components of a nucleic acid expression system are delivered concurrently. In some embodiments, components of a nucleic acid expression system are delivered separately (e.g., sequentially). In some embodiments, an oligonucleotide comprising a sequence that encodes a helper polypeptide is delivered to a cell, and an oligonucleotide comprising a payload sequence is delivered at a later time. In some embodiments, an oligonucleotide comprising a payload sequence is delivered 30 min, 1 hour, 2 hours, 3 hours, 4 hours, 5 hours, 6 hours, 12 hours, 18 hours, 1 day, 2 days, 3 days, 4 days, 5 days, 6 days, 1 week, 2 weeks, 3 weeks, 4 weeks, 6 weeks, or 8 weeks after an oligonucleotide comprising a sequence that encodes a helper polypeptide is delivered.

In some embodiments, a nucleic acid expression system of the present disclosure can be used for oligonucleotide delivery to a target cell for a gene therapy in a subject. In some embodiments, a nucleic acid expression system of the present disclosure can be used for oligonucleotide delivery to a target cell is isolated from a subject. In some embodiments, a target cell can be autologous to a subject (i.e., from a subject). In some embodiments, a target cell can be non-autologous (i.e., allogeneic or xenogenic) to a subject.

In some embodiments, components of a nucleic acid expression system described herein (e.g., an oligonucleotide comprising a payload and an oligonucleotide comprising a sequence that encodes a helper polypeptide) are administered to a subject. In some embodiments, components of a nucleic acid expression system are administered concurrently. In some embodiments, components of a nucleic acid expression system are administered separately. In some embodiments, an oligonucleotide comprising a sequence that encodes a helper polypeptide is administered to a subject, and an oligonucleotide comprising a payload sequence is administered to a subject at a later time. In some embodiments, an oligonucleotide comprising a payload sequence is administered to a subject 30 min, 1 hour, 2 hours, 3 hours, 4 hours, 5 hours, 6 hours, 12 hours, 18 hours, 1 day, 2 days, 3 days, 4 days, 5 days, 6 days, 1 week, 2 weeks, 3 weeks, 4 weeks, 6 weeks, or 8 weeks after an oligonucleotide comprising a sequence that encodes a helper polypeptide is administered.

In some embodiments, a composition comprising at least one oligonucleotide sequences that encodes a helper polypeptide is administered to a subject that has been or is to be administered an oligonucleotide comprising a payload, such that they receive both.

In some embodiments, a composition comprising an oligonucleotide comprising a payload is administered to a subject that has been or is to be administered at least one oligonucleotide sequence that encodes a helper polypeptide, such that they receive both.

Cells

Cells comprising any embodiment of a nucleic acid expression system described herein are also provided herein. For example, in some embodiments, a cell comprises an RNA oligonucleotide comprising a payload sequence and an RNA oligonucleotide comprising a sequence that encodes a helper polypeptide (e.g., ones as described herein).

Any cells can be chosen to express a payload sequence delivered via an oligonucleotide (e.g., DNA oligonucleotide or RNA oligonucleotide). In some embodiments, cells to be contacted with any of compositions or nucleic acid expression systems described herein can be wild-type cells, normal cells, diseased cells, or transgenic cells. In some embodiments, cells to be contacted with any of compositions or nucleic acid expression systems described herein can be eukaryotic cells (e.g., mammalian cells).

In some embodiments, cells as provided herein are cells that have been previously treated at least once or more (e.g., 1, 2, 3, 4, 5, 6, 7, 8, 9, 10 times or more) with one or more oligonucleotides. In some embodiments, oligonucleotides that are previously introduced into cells are DNA oligonucleotides. In some embodiments, oligonucleotides that are previously introduced into cells are RNA oligonucleotides (e.g., mRNA oligonucleotides).

### IV. Methods of Uses

The present disclosure provides, among other things, methods for using nucleic acid expression systems in the context of the present disclosure or components thereof. The present disclosure recognizes that challenges associated with cell treatment based on oligonucleotides involve high degradation of DNA oligonucleotides in cytoplasm and/or high immunogenicity associated with foreign RNA oligonucleotides to be introduced into cells. The present disclosure, among other things, also recognizes that while using non-standard base chemistries may reduce immunogenicity of mRNA therapeutics, such modification may adversely affect efficiencies of translating mRNA to corresponding peptides or polypeptides in cells. Further, concerns with residual immune response that precludes repeated dosing and/or high-level dosing still remain. Therefore, there remains a need in the field for methods of effectively delivering oligonucleotides to target cells that increases expression and/or activity of a payload polynucleotide in target cells. For example, there remains a need in the field for methods of delivering RNA oligonucleotides to target cells that minimize activation of myriad innate immune sensors while are still efficiently recognized by translational machinery.

The present disclosure, at least in part, addresses this need and provides methods that comprise contacting a target cell with an oligonucleotide (e.g., DNA or RNA oligonucleotide) comprising a payload sequence and an oligonucleotide comprising a sequence that encodes a helper polypeptide (e.g., ones described herein).

Any payload sequences and/or any helper polypeptides disclosed herein may be used in the methods described herein. For example, a helper polypeptide may include a nuclear localization signal (NLS) polypeptide, a DNA mimic polypeptide, an immunomodulatory polypeptide (e.g., a modulator of innate immunity), or a synthetic cell surface receptor polypeptide, such as described herein.

Accordingly, methods for using any embodiment of nucleic acid expression systems, compositions, and pharmaceutical compositions described herein are provided. In some embodiments, a method comprises (i) contacting a target cell with a DNA oligonucleotide comprising a payload sequence; and (ii) contacting the target cell with a DNA oligonucleotide comprising a sequence that encodes a helper polypeptide (e.g., ones described herein). In some embodiments, a method comprises (i) contacting a target cell with a DNA oligonucleotide comprising a payload sequence; and (ii) contacting the target cell with an RNA (e.g., mRNA) oligonucleotide comprising a sequence that encodes a helper polypeptide (e.g., ones described herein). In some embodiments, a method comprises (i) contacting a target cell with an RNA (e.g., mRNA) oligonucleotide comprising a payload sequence; and (ii) contacting the target cell with an RNA (e.g., mRNA) oligonucleotide comprising a sequence that encodes a helper polypeptide (e.g., ones described herein). In some embodiments, oligonucleotides (e.g., comprising a payload sequence and/or comprising a sequence that encodes a helper polypeptide) are synthetic oligonucleotides (e.g., synthetic DNA oligonucleotides or synthetic RNA oligonucleotides).

In some embodiments, methods described herein are for enhancing expression and/or activity of a payload sequence in a target cell when the payload sequence is introduced into the target cell in the presence of an oligonucleotide (e.g., a DNA or RNA oligonucleotide) comprising a sequence that encodes a helper polypeptide (e.g., ones described herein). In some embodiments, expression and/or activity of a payload sequence in a target cell is enhanced by at least 30% or more, including, e.g., at least 40%, at least 50%, at least 60%, at least 70%, at least 80%, at least 90%, at least 95%, or more, as compared to expression and/or activity of the same payload sequence in the target cell in the absence of an oligonucleotide (e.g., DNA or RNA oligonucleotide) comprising a sequence that encodes a helper polypeptide (e.g., ones described herein). In some embodiments, expression and/or activity of a payload sequence in a target cell is enhanced by at least 1.1-fold or more, including, e.g., at least 1.5-fold, at least 2-fold, at least 2.5-fold, at least 3-fold, at least 3.5-fold, at least 4-fold, at least 5-fold, at least 10-fold, at least 20-fold, at least 30-fold, at least 40-fold, or more, as compared to expression and/or activity of the same payload sequence in the target cell in the absence of an oligonucleotide (e.g., DNA or RNA oligonucleotide) comprising a sequence that encodes a helper polypeptide (e.g., ones described herein). Accordingly, in some embodiments, provided herein is a method for enhancing expression and/or activity of a payload sequence delivered via an oligonucleotide, wherein the method comprises (a) contacting a target cell with an oligonucleotide (e.g., DNA or RNA oligonucleotide) comprising a payload sequence; and (b) contacting the target cell with an oligonucleotide (e.g., DNA or RNA oligonucleotide) comprising a sequence that encodes a helper polypeptide (e.g., ones described herein).

In some embodiments, methods described herein are for enhancing nuclear localization of an oligonucleotide (e.g., a DNA or RNA oligonucleotide) comprising a payload sequence in a target cell when the payload sequence is introduced into the target cell in the presence of an oligonucleotide (e.g., a DNA or RNA oligonucleotide) comprising a sequence that encodes a helper polypeptide (e.g., ones described herein). In some embodiments, nuclear localization of an oligonucleotide comprising a payload sequence in a target cell is enhanced by at least 30% or more, including, e.g., at least 40%, at least 50%, at least 60%, at least 70%, at least 80%, at least 90%, at least 95%, or more, as compared to nuclear localization of an oligonucleotide comprising a payload sequence in a target cell in the absence of an oligonucleotide (e.g., DNA or RNA oligonucleotide) comprising a sequence that encodes a helper polypeptide (e.g., ones described herein). In some embodiments, nuclear localization of an oligonucleotide comprising a payload sequence in a target cell is enhanced by at least 1.1-fold or more, including, e.g., at least 1.5-fold, at least 2-fold, at least 2.5-fold, at least 3-fold, at least 3.5-fold, at least 4-fold, at least 5-fold, at least 10-fold, at least 20-fold, at least 30-fold, at least 40-fold, or more, as compared to nuclear localization of an oligonucleotide comprising the same payload sequence in a target cell in the absence of an oligonucleotide (e.g., DNA or RNA oligonucleotide) comprising a sequence that encodes a helper polypeptide (e.g., ones described herein). Accordingly, in some embodiments, provided herein is a method for enhancing nuclear localization of an oligonucleotide comprising a payload sequence in a target cell, wherein the method comprises (a) contacting a target cell with an oligonucleotide (e.g., DNA or RNA oligonucleotide) comprising a payload sequence; and (b) contacting the target cell with an oligonucleotide (e.g., DNA or RNA oligonucleotide) comprising a sequence that encodes a helper polypeptide (e.g., ones described herein).

In some embodiments, methods described herein are for enhancing persistence or uptake of an oligonucleotide (e.g., a DNA or RNA oligonucleotide) comprising a payload sequence in a target cell when the payload sequence is introduced into the target cell in the presence of an oligonucleotide (e.g., a DNA or RNA oligonucleotide) comprising a sequence that encodes a helper polypeptide (e.g., ones described herein). In some embodiments, persistence or uptake of an oligonucleotide comprising a payload sequence in a target cell is enhanced by at least 30% or more, including, e.g., at least 40%, at least 50%, at least 60%, at least 70%, at least 80%, at least 90%, at least 95%, or more, as compared to persistence or uptake of an oligonucleotide comprising the same payload sequence in a target cell in the absence of an oligonucleotide (e.g., DNA or RNA oligonucleotide) comprising a sequence that encodes a helper polypeptide (e.g., ones described herein). In some embodiments, persistence or uptake of an oligonucleotide comprising a payload sequence in a target cell is enhanced by at least 1.1-fold or more, including, e.g., at least 1.5-fold, at least 2-fold, at least 2.5-fold, at least 3-fold, at least 3.5-fold, at least 4-fold, at least 5-fold, at least 10-fold, at least 20-fold, at least 30-fold, at least 40-fold, or more, as compared to persistence or uptake of an oligonucleotide comprising the same payload sequence in a target cell in the absence of an oligonucleotide (e.g., DNA or RNA oligonucleotide) comprising a sequence that encodes a helper polypeptide (e.g., ones described herein). Accordingly, in some embodiments, provided herein is a method for enhancing persistence or uptake of an oligonucleotide comprising a payload sequence in a target cell comprising a payload sequence in a target cell, wherein the method comprises (a) contacting a target cell with an oligonucleotide (e.g., DNA or RNA oligonucleotide) comprising a payload sequence; and (b) contacting the target cell with an oligonucleotide (e.g., DNA or RNA oligonucleotide) comprising a sequence that encodes a helper polypeptide (e.g., ones described herein).

In some embodiments, methods described herein are for enhancing viability of a target cell upon contacting with an oligonucleotide (e.g., DNA or RNA oligonucleotide) comprising a payload sequence and an oligonucleotide (e.g., DNA or RNA oligonucleotide) comprising a sequence that encodes a helper polypeptide (e.g., ones described herein). In some embodiments, viability of a target cell upon contacting with an oligonucleotide (e.g., DNA or RNA oligonucleotide) comprising a payload sequence and an oligonucleotide (e.g., DNA or RNA oligonucleotide) comprising a sequence that encodes a helper polypeptide is enhanced by at least 30% or more, including, e.g., at least 40%, at least 50%, at least 60%, at least 70%, at least 80%, at least 90%, at least 95%, or more, as compared to viability of a target cell upon contacting with an oligonucleotide (e.g., DNA or RNA oligonucleotide) comprising the same payload sequence in the absence of an oligonucleotide comprising a sequence that encodes a helper polypeptide. In some embodiments, viability of a target cell upon contacting with an oligonucleotide (e.g., DNA or RNA oligonucleotide) comprising a payload sequence and an oligonucleotide (e.g., DNA or RNA oligonucleotide) comprising a sequence that encodes a helper polypeptide is enhanced by at least 1.1-fold or more, including, e.g., at least 1.5-fold, at least 2-fold, at least 2.5-fold, at least 3-fold, at least 3.5-fold, at least 4-fold, at least 5-fold, at least 10-fold, at least 20-fold, at least 30-fold, at least 40-fold, or more, as compared to viability of a target cell upon contacting with an oligonucleotide (e.g., DNA or RNA oligonucleotide) comprising the same payload sequence in the absence of an oligonucleotide comprising a sequence that encodes a helper polypeptide. Accordingly, in some embodiments, provided herein is a method for enhancing viability of a target cell upon introduction of a payload sequence via an oligonucleotide, wherein the method comprises (a) contacting a target cell with an oligonucleotide (e.g., DNA or RNA oligonucleotide) comprising a payload sequence; and (b) contacting the target cell with an oligonucleotide (e.g., DNA or RNA oligonucleotide) comprising a sequence that encodes a helper polypeptide (e.g., ones described herein).

In some embodiments, methods described herein are for reducing non-specific toxicity induced in a target cell by introduction of an oligonucleotide (e.g., DNA or RNA oligonucleotide) comprising a payload sequence when the payload sequence is introduced into the target cell in the presence of an oligonucleotide (e.g., DNA or RNA oligonucleotide) comprising a sequence that encodes a helper polypeptide. In some embodiments, non-specific toxicity induced in a target cell by introduction of an oligonucleotide (e.g., DNA or RNA oligonucleotide) comprising a payload sequence is reduced by at least 30% or more, including, e.g., at least 40%, at least 50%, at least 60%, at least 70%, at least 80%, at least 90%, at least 95%, at least 98%, at least 99% or more, as compared to non-specific toxicity induced in a target cell by an oligonucleotide comprising the same payload sequence in the absence of an oligonucleotide comprising a sequence that encodes a helper polypeptide. Accordingly, in some embodiments, provided herein is a method for reducing non-specific cell toxicity induced by introduction of a payload sequence via an oligonucleotide, wherein the method comprises (a) contacting a target cell with an oligonucleotide (e.g., DNA or RNA oligonucleotide) comprising a payload sequence; and (b) contacting the target cell with an oligonucleotide comprising a sequence that encodes a helper polypeptide (e.g., ones described herein).

In some embodiments, provided herein are methods by which innate immunity-triggered suppression of protein translation, mRNA degradation, and non-specific toxicity induced by RNA oligonucleotides are reduced, thereby enhancing expression of RNA oligonucleotides in cells. Further, higher doses and/or repeated doses of RNA oligonucleotides can be applied to cells using any of methods described herein to improve or sustain expression of RNA oligonucleotides without adversely inducing non-specific cell toxicity that would otherwise generally induced by any RNA oligonucleotides. These advantages can be beneficial for delivering and improving the effectiveness of RNA therapeutics and vaccines.

In some embodiments where a payload oligonucleotide is an RNA oligonucleotide, methods described herein are for reducing innate immunity-triggered suppression of protein translation when a payload sequence is introduced into a target cell in the presence of an RNA oligonucleotide (e.g., a mRNA oligonucleotide) encoding a helper polypeptide, e.g., an immunomodulatory polypeptide such as a modulator of innate immunity (e.g., ones described herein). In some embodiments, innate immunity-triggered suppression of protein translation of an introduced payload sequence is reduced by at least 30% or more, including, e.g., at least 40%, at least 50%, at least 60%, at least 70%, at least 80%, at least 90%, at least 95%, at least 98%, at least 99% or more, as compared to innate immunity-triggered suppression of protein translation of an introduced payload sequence in the absence of an RNA oligonucleotide comprising a sequence that encodes a helper polypeptide, e.g., an immunomodulatory polypeptide such as a modulator of innate immunity (e.g., ones described herein). Accordingly, in some embodiments, provided herein is a method for reducing innate immunity-triggered suppression of translating an introduced mRNA payload oligonucleotide into a corresponding payload peptide or polypeptide, wherein the method comprises (a) contacting a target cell with an RNA oligonucleotide comprising a payload sequence; and (b) contacting the target cell with an RNA oligonucleotide comprising a sequence that encodes a helper polypeptide, e.g., an immunomodulatory polypeptide such as a modulator of innate immunity (e.g., ones described herein).

In some embodiments where a payload oligonucleotide is an RNA oligonucleotide, methods described herein are for reducing innate immunity-triggered mRNA degradation when a payload sequence encoded by a mRNA oligonucleotide is introduced into a target cell in the presence of an RNA oligonucleotide (e.g., a mRNA oligonucleotide) encoding a helper polypeptide, e.g., an immunomodulatory polypeptide such as a modulator of innate immunity (e.g., ones described herein). In some embodiments, innate immunity-triggered degradation of an introduced mRNA payload oligonucleotide is reduced by at least 30% or more, including, e.g., at least 40%, at least 50%, at least 60%, at least 70%, at least 80%, at least 90%, at least 95%, at least 98%, at least 99% or more, as compared to innate immunity-triggered degradation of an introduced mRNA payload oligonucleotide in the absence of an RNA oligonucleotide comprising a sequence that encodes a helper polypeptide, e.g., an immunomodulatory polypeptide such as a modulator of innate immunity (e.g., ones described herein). Accordingly, in some embodiments, provided herein is a method for reducing innate immunity-triggered degradation of an introduced mRNA payload oligonucleotide, wherein the method comprises (a) contacting a target cell with an RNA oligonucleotide comprising a payload sequence; and (b) contacting the target cell with an RNA oligonucleotide comprising a sequence that encodes a helper polypeptide, e.g., an immunomodulatory polypeptide such as a modulator of innate immunity (e.g., ones described herein).

Methods described herein can be used for in vitro, ex vivo and in vivo applications. Thus, cells to which oligonucleotides (e.g., an oligonucleotide comprising a payload sequence and/or an oligonucleotide comprising a sequence that encodes a helper polypeptide) are delivered can be, for example, cells cultured in vitro or ex vivo, cells within a tissue, or cells present in a subject or organism. In some embodiments, cells to which oligonucleotides (e.g., an oligonucleotide comprising a payload sequence and/or an oligonucleotide comprising a sequence that encodes a helper polypeptide) are delivered can be cells that have been previously treated at least once or more (e.g., 1, 2, 3, 4, 5, 6, 7, 8, 9, 10 times or more) with one or more oligonucleotides. In some embodiments, oligonucleotides that are previously introduced into cells can be DNA oligonucleotides. In some embodiments, oligonucleotides that are previously introduced into cells can be RNA oligonucleotides (e.g., mRNA oligonucleotides).

Oligonucleotides (e.g., an oligonucleotide comprising a payload sequence and/or an oligonucleotide comprising a sequence that encodes a helper polypeptide) used in any methods described herein can be delivered to cells by any of known methods in the art, including, but not limited to, transfection into cells (e.g., via electroporation, chemical methods, etc.), delivery via particles (e.g., nanoparticles or liposomes), and/or administration to an organism (e.g., by any suitable administration route).

In some embodiments, cells subjected to a method described herein are present in a subject. Therefore, in these embodiments, a target cell present in a subject is contacted with an oligonucleotide comprising a payload sequence by administering the oligonucleotide comprising a payload sequence to the subject. In some embodiments, a target cell present in a subject is contacted with an oligonucleotide comprising a sequence that encodes a helper polypeptide (e.g., ones described herein) by administering the oligonucleotide comprising a sequence that encodes a helper polypeptide to the subject.

In some embodiments, methods, nucleic acid expression systems, and compositions described herein can be used for delivering an oligonucleotide (e.g., DNA or RNA oligonucleotide) to a target cell for a gene therapy or RNA oligonucleotide therapy in a subject. In some embodiments, a target cell to be subjected to a method, nucleic acid expression system, and/or composition described herein is isolated from a subject. In some embodiments, a target cell can be autologous to a subject (i.e., from a subject). In some embodiments, a target cell can be non-autologous (i.e., allogeneic or xenogenic) to a subject.

In some embodiments, a target cell (e.g., for in vitro, ex vivo, or in vivo applications described herein) is contacted with an oligonucleotide (e.g., DNA or RNA oligonucleotide) comprising a payload sequence and an oligonucleotide (e.g., DNA or RNA oligonucleotide) comprising a sequence that encodes a helper polypeptide concurrently. In some embodiments, a target cell (e.g., for in vitro, ex vivo, or in vivo applications described herein) is contacted with an oligonucleotide (e.g., DNA or RNA oligonucleotide) comprising a payload sequence and an oligonucleotide (e.g., DNA or RNA oligonucleotide) comprising a sequence that encodes a helper polypeptide separately. For example, in some embodiments, an oligonucleotide (e.g., DNA or RNA oligonucleotide) comprising a payload sequence and an oligonucleotide (e.g., DNA or RNA oligonucleotide) comprising a sequence that encodes a helper polypeptide (e.g., ones described herein) are delivered to a target cell within 5 mins, 10 mins, 15 mins, 20 mins, 25 mins, 30 mins, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 1 week, 2 weeks, 3 weeks, 4 weeks, 6 weeks, or 8 weeks. For example, in some embodiments, an oligonucleotide (e.g., DNA or RNA oligonucleotide) comprising a sequence that encodes a helper polypeptide is delivered to a target cell, and an oligonucleotide (e.g., DNA or RNA oligonucleotide) comprising a payload sequence is delivered to the target cell at a later time. For example, in some embodiments, an oligonucleotide (e.g., DNA or RNA oligonucleotide) comprising a payload sequence is delivered to a target cell 30 min, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 1 week, 2 weeks, 3 weeks, 4 weeks, 6 weeks, or 8 weeks after an oligonucleotide (e.g., DNA or RNA oligonucleotide) comprising a sequence that encodes a helper polypeptide is delivered. In some embodiments, an oligonucleotide (e.g., DNA or RNA oligonucleotide) comprising a payload sequence is delivered to a target cell during a time when innate immunity pathway is attenuated (e.g., temporarily attenuated by at least 10% or more including, e.g., at least 20%, at least 30%, at least 40%, or more) by an oligonucleotide (e.g., DNA or RNA oligonucleotide) comprising a sequence that encodes a helper polypeptide, e.g., an immunomodulatory polypeptide such as a modulator of innate immunity (e.g., ones described herein).

In some embodiments, a composition comprising at least one oligonucleotide (e.g., DNA or RNA oligonucleotide) comprising a sequence that encodes a helper polypeptide is delivered to a target cell that has been contacted with an oligonucleotide (e.g., DNA or RNA oligonucleotide) comprising a payload sequence, such that the target cell receives both.

In some embodiments, a composition comprising an oligonucleotide (e.g., DNA or RNA oligonucleotide) comprising a payload sequence is administered to a target cell that has been contacted with at least one oligonucleotide (e.g., DNA or RNA oligonucleotide) comprising a sequence that encodes a helper polypeptide, such that the target cell receives both.

In some embodiments, provided are methods for enhancing expression of an oligonucleotide in a target cell, the method including: administering an oligonucleotide comprising a payload sequence; and administering at least one oligonucleotide comprising a sequence that encodes a helper polypeptide.

In some embodiments, provided are methods for enhancing expression of an oligonucleotide in a target cell, the method including: administering an oligonucleotide comprising a payload sequence; and administering at least one mRNA oligonucleotide sequence that encodes a helper polypeptide.

In some embodiments, provided are methods for enhancing expression of an oligonucleotide in a target cell, the method including: administering an oligonucleotide comprising a payload sequence; and administering a composition that delivers a helper polypeptide.

In some embodiments, provided are methods for increasing nuclear localization of an oligonucleotide comprising, the method including: administering an oligonucleotide comprising a payload sequence; and administering an oligonucleotide sequence that encodes a helper polypeptide for enhancing nuclear import of the oligonucleotide comprising a payload sequence in a target cell.

In some embodiments, provided are methods for increasing nuclear localization of an oligonucleotide comprising, the method including: administering a DNA oligonucleotide comprising a payload sequence; and administering an at least one mRNA oligonucleotide sequence that encodes a helper polypeptide for enhancing nuclear import of the oligonucleotide comprising a payload sequence in a target cell.

In some embodiments, provided methods include: administering an oligonucleotide comprising a payload sequence; and administering an at least one oligonucleotide sequence that encodes a helper polypeptide comprising a nuclear localization signal (NLS) polypeptide, and an oligonucleotide encoding a DNA-binding domain (DBD) polypeptide.

In some embodiments, provided are methods for enhancing persistence or uptake of an oligonucleotide comprising, the method including: administering an oligonucleotide comprising a payload sequence; and administering an oligonucleotide sequence that encodes a helper polypeptide for enhancing persistence or uptake of the oligonucleotide comprising a payload sequence in a target cell.

In some embodiments, provided are methods for enhancing persistence or uptake of an oligonucleotide comprising, the method including: administering a DNA oligonucleotide comprising a payload sequence; and administering an at least one mRNA oligonucleotide sequence that encodes a helper polypeptide for enhancing persistence or uptake of the oligonucleotide comprising a payload sequence in a target cell.

In some embodiments, an oligonucleotide comprising a payload sequence and the at least one oligonucleotide comprising a sequence that encodes a helper polypeptide are administered sequentially. In some embodiments, an oligonucleotide comprising a payload sequence and the at least one oligonucleotide comprising a sequence that encodes a helper polypeptide are administered concurrently. In some embodiments, an oligonucleotide comprising a payload sequence and at least one oligonucleotide comprising a sequence that encodes a helper polypeptide are part of a vector.

The following embodiments as described below are also within the scope of the disclosures:

A nucleic acid expression system comprising: (i) an RNA oligonucleotide comprising a payload sequence, and (ii) an RNA oligonucleotide comprising a sequence that encodes a US11 polypeptide.

The nucleic acid expression system of paragraph 254, wherein the RNA oligonucleotide of (i) is a synthetic RNA oligonucleotide.

The nucleic acid expression system of paragraph 254 or 255, wherein the RNA oligonucleotide of (ii) is a synthetic RNA oligonucleotide.

The nucleic acid expression system of any one of paragraphs 254-256, wherein the RNA oligonucleotide of (i) is a messenger RNA (mRNA) oligonucleotide.

The nucleic acid expression system of any one of paragraphs 254-257, wherein the RNA oligonucleotide of (ii) is a mRNA oligonucleotide.

The nucleic acid expression system of any one of paragraphs 254-258, wherein the US11 polypeptide is or includes an RNA binding domain of a US11 polypeptide.

The nucleic acid expression system of any one of paragraphs 254-259, wherein the US11 polypeptide comprises the sequence of SEQ ID NO.: 1 or SEQ ID NO: 2.

A composition comprising the nucleic acid expression system of any one of paragraphs 254-260.

The composition of paragraph 261, wherein the composition is a pharmaceutical composition.

The composition of paragraph 262, further comprising a pharmaceutically acceptable carrier.

A cell comprising the nucleic acid expression system of any one of paragraphs 254-260.

The cell of paragraph 264, wherein the cell is a diseased cell.

A method comprising: (a) contacting a target cell with an RNA oligonucleotide comprising a payload sequence; and (b) contacting the target cell with an RNA oligonucleotide comprising a sequence that encodes a US11 polypeptide.

The method of paragraph 266, wherein the RNA oligonucleotide comprising the payload sequence is a synthetic RNA oligonucleotide.

The method of paragraph 266 or 267, wherein the RNA oligonucleotide comprising the sequence that encodes the US11 polypeptide is a synthetic RNA oligonucleotide.

The method of any one of paragraphs 266-268, wherein the RNA oligonucleotide comprising the payload sequence is a messenger RNA (mRNA) oligonucleotide.

The method of any one of paragraphs 266-269, wherein the RNA oligonucleotide comprising the sequence that encodes the US11 polypeptide is a mRNA oligonucleotide.

The method of any one of paragraphs 266-270, wherein the US11 polypeptide is or includes an RNA binding domain of a US11 polypeptide.

The method of any one of paragraphs 266-271, wherein the US11 polypeptide comprises the sequence of SEQ ID NO.: 1 or SEQ ID NO: 2.

The method of any one of paragraphs 266-272, wherein the method is for enhancing expression and/or activity of the payload sequence in the target cell.

The method of paragraph 273, wherein the expression and/or activity of the payload sequence in the target cell is enhanced by at least 30% or more, as compared to the expression and/or activity of the payload sequence in the target cell in the absence of the RNA oligonucleotide comprising the sequence that encodes the US11 polypeptide.

The method of any one of paragraphs 266-274, wherein the method is for enhancing viability of the target cell upon contacting with the RNA oligonucleotide comprising the payload sequence and the RNA oligonucleotide comprising the sequence that encodes the US11 polypeptide.

The method of paragraph 275, wherein the viability of the target cell upon contacting with the RNA oligonucleotide comprising the payload sequence and the RNA oligonucleotide comprising the sequence that encodes the US11 polypeptide is enhanced by at least 30% or more, as compared to the viability of the target cell upon contacting with the RNA oligonucleotide comprising the payload sequence in the absence of the RNA oligonucleotide comprising the sequence that encodes the US11 polypeptide.

The method of any one of paragraphs 266-276, wherein the method is for reducing non-specific toxicity induced in the target cell by the RNA oligonucleotide comprising the payload sequence.

The method of paragraph 277, wherein the non-specific toxicity induced in the target cell by the RNA oligonucleotide comprising the payload sequence is reduced by at least 30% or more, as compared to the non-specific toxicity induced in the target cell by the RNA oligonucleotide comprising the payload sequence in the absence of the RNA oligonucleotide comprising the sequence that encodes the US11 polypeptide.

The method of any one of paragraphs 266-278, wherein the target cell is previously contacted at least once by one or more oligonucleotides.

The method of any one of paragraphs 266-279, wherein the target cell is contacted with the RNA oligonucleotide comprising the payload sequence and the RNA oligonucleotide comprising the sequence that encodes the US11 polypeptide concurrently.

The method of any one of paragraphs 266-279, wherein the target cell is contacted with the RNA oligonucleotide comprising the payload sequence and the RNA oligonucleotide comprising the sequence that encodes the US11 polypeptide separately.

The method of paragraph 281, wherein the target cell is contacted with the RNA oligonucleotide comprising the payload sequence and the RNA oligonucleotide comprising the sequence that encodes the US11 polypeptide separately within 24 hours or less.

The method of any one of paragraphs 266-282, wherein the target cell is present in a subject.

The method of paragraph 283, wherein the target cell present in the subject is contacted with the RNA oligonucleotide comprising the payload sequence by administering the RNA oligonucleotide comprising the payload sequence to the subject.

The method of paragraph 283 or 284, wherein the target cell present in the subject is contacted with the RNA oligonucleotide comprising the sequence that encodes the US11 polypeptide by administering the RNA oligonucleotide comprising encoding the US11 polypeptide to the subject.

The method of any one of paragraphs 266-285, wherein the target cell is a diseased cell.

Further embodiments that are also within the scope of the disclosures are provided below:

1. A nucleic acid expression system comprising:

(i) an oligonucleotide comprising a payload sequence, and

(ii) at least one oligonucleotide comprising a sequence that encodes a helper polypeptide for enhancing expression of the oligonucleotide comprising a payload sequence in a target cell.

2. The nucleic acid expression system of embodiment 1, wherein the helper polypeptide is or comprises one or more of the following: a nuclear localization signal (NLS) polypeptide, a DNA mimic polypeptide, a modulator of innate immunity, and a synthetic cell surface receptor polypeptide.
3. The nucleic acid expression system of embodiment 1 or 2, wherein the at least one oligonucleotide comprising a sequence that encodes a helper polypeptide is a synthetic oligonucleotide.
4. The nucleic acid expression system of any one of embodiments 1-3, wherein the at least one oligonucleotide comprising a sequence that encodes a helper polypeptide is a DNA oligonucleotide.
5. The nucleic acid expression system of any one of embodiments 1-3, wherein the at least one oligonucleotide comprising a sequence that encodes a helper polypeptide is a RNA oligonucleotide (e.g., a messenger RNA (mRNA) oligonucleotide).
6. The nucleic acid expression system of any one of embodiments 1-5, wherein the oligonucleotide comprising a payload sequence is a synthetic oligonucleotide.
7. The nucleic acid expression system of any one of embodiments 1-6, wherein the oligonucleotide comprising a payload sequence is a DNA oligonucleotide.
8. The nucleic acid expression system of any one of embodiments 1-6, wherein the oligonucleotide comprising a payload sequence is an RNA oligonucleotide (e.g., a mRNA oligonucleotide).
9. The nucleic acid expression system of any one of embodiments 1-8, wherein the oligonucleotide comprising a payload sequence and/or the at least one oligonucleotide comprising a sequence that encodes a helper polypeptide are part of a vector.
10. A nucleic acid expression system comprising:

(i) a synthetic DNA oligonucleotide comprising a payload sequence, and

(ii) at least one RNA oligonucleotide (e.g., at least one mRNA oligonucleotide) comprising a sequence that encodes a helper polypeptide for enhancing expression of the synthetic DNA oligonucleotide comprising a payload sequence in a target cell.

11. The nucleic acid expression system of embodiment 10, wherein the helper polypeptide is or comprises one or more of the following: a nuclear localization signal (NLS) polypeptide, a DNA mimic polypeptide, a modulator of innate immunity, and a synthetic cell surface receptor polypeptide.
12. The nucleic acid expression system of any one of embodiments 1-11, wherein the oligonucleotide comprising a payload sequence comprises homology arms.
13. The nucleic acid expression system of any one of embodiments 1-12, wherein the expression system further comprises an oligonucleotide comprising a sequence that encodes a targeted nuclease.
14. The nucleic acid expression system of embodiment 13, wherein the oligonucleotide comprising a sequence that encodes a targeted nuclease is a DNA oligonucleotide.
15. The nucleic acid expression system of embodiment 13, wherein the oligonucleotide comprising a sequence that encodes a targeted nuclease is an RNA oligonucleotide (e.g., a mRNA oligonucleotide).
16. The nucleic acid expression system of any one of embodiments 13-15, wherein the targeted nuclease is a zinc-finger nuclease (ZFN), TAL effector domain nuclease (TALEN), or an engineered CRISPR/Cas9 system.
17. A nucleic acid expression system that includes (i) an oligonucleotide comprising a payload sequence and (ii) a composition that delivers at least one helper polypeptide.
18. The nucleic acid expression system of embodiment 17, wherein the at least one helper polypeptide is or comprises one or more of the following: a nuclear localization signal (NLS) polypeptide, a DNA mimic polypeptide, a modulator of innate immunity, and a synthetic cell surface receptor polypeptide.
19. The nucleic acid expression system of embodiment 17 or 18, wherein the composition that delivers a helper polypeptide is or comprises (i) an oligonucleotide comprising a sequence that encodes a helper polypeptide and/or (ii) a helper polypeptide.
20. The nucleic acid expression system of any one of embodiments 17-19, wherein the oligonucleotide comprising a payload sequence comprises homology arms.
21. The nucleic acid expression system of any one of embodiments 17-20, wherein the expression system further comprises a composition that delivers a targeted nuclease.
22. The nucleic acid expression system of embodiment 21, wherein the composition that delivers a targeted nuclease is or comprises (i) an oligonucleotide comprising a sequence that encodes a targeted nuclease and/or (ii) a targeted nuclease polypeptide.
23. The nucleic acid expression system of embodiment 21 or 22, wherein the targeted nuclease is a zinc-finger nuclease (ZFN), TAL effector domain nuclease (TALEN), or an engineered CRISPR/Cas9 system.
24. The nucleic acid expression system of any one of embodiments 1-23, wherein the helper polypeptide is or comprises a NLS polypeptide.
25. The nucleic acid expression system of embodiment 24, wherein the NLS polypeptide is an SV40 NLS polypeptide or variant thereof.
26. The nucleic acid expression system of embodiment 24, wherein the NLS polypeptide is from EGL-13 polypeptide, c-Myc polypeptide, NLP polypeptide or TUS polypeptide.
27. The nucleic acid expression system of any one of embodiments 24-26, wherein the NLS polypeptide is operatively connected to a DNA-binding domain (DBD) polypeptide.
28. The nucleic acid expression system of embodiment 27, wherein the DBD polypeptide is not regulated by a small molecule.
29. The nucleic acid expression system of embodiment 28, wherein the DBD polypeptide is or comprises a Cro repressor polypeptide or a catalytically-inactive meganuclease variant.
30. The nucleic acid expression system of embodiment 27, wherein the DBD polypeptide is a synthetic DBD polypeptide.
31. The nucleic acid expression system of embodiment 30, wherein the DBD polypeptide is or comprises a zinc finger polypeptide, a TAL domain polypeptide, or a catalytically-inactive Cas9 polypeptide.
32. The nucleic acid expression system of embodiment 27, wherein the DBD polypeptide is a non-specific DBD polypeptide.
33. The nucleic acid expression system of embodiment 32, wherein the DBD polypeptide is or comprises Sso7d polypeptide, H-NS polypeptide, HU-1 polypeptide, HU-2 polypeptide, p6 polypeptide of 429, A104R polypeptide of ASFV, dsp polypeptide, TmHU polypeptide, HPhA polypeptide, or HCcp3 polypeptide.
34. The nucleic acid expression system of any one of embodiments 27-33, wherein the NLS polypeptide and DBD polypeptide forms a fusion polypeptide.
35. The nucleic acid expression system of any one of embodiments 27-33, wherein the NLS polypeptide and DBD polypeptide are separate polypeptides that can dimerize.
36. The nucleic acid expression system of embodiment 35, wherein the NLS polypeptide and the DBD polypeptide dimerize through inducible dimerization domains.
37. The nucleic acid expression system of embodiment 36, wherein the inducible dimerization domain is a rapamycin-inducible FRB/FKBP pair.
38. The nucleic acid expression system of any one of embodiments 1-37, wherein the helper polypeptide is or comprises a DNA mimic polypeptide.
39. The nucleic acid expression system of embodiment 38, wherein the DNA mimic polypeptide is selected from any one of Ocr polypeptide, ArdA polypeptide, NuiA polypeptide, HI1450 polypeptide, DMP12 polypeptide, MfpA polypeptide, Arn polypeptide, Gam polypeptide and variants thereof.
40. The nucleic acid expression system of any one of embodiments 1-39, wherein the helper polypeptide is or comprises a modulator of innate immunity.
41. The nucleic acid expression system of embodiment 40, wherein the modulator of innate immunity is selected from any one of viral interferon regulatory factor 1 (vIRF1) polypeptide, ORF52/KicGAS polypeptide, PLP2-TM polypeptide, PLP2 polypeptide, US11 polypeptide, and variants thereof.
42. The nucleic acid expression system of any one of embodiments 1-41, wherein the helper polypeptide is or comprises a synthetic cell surface receptor polypeptide.
43. The nucleic acid expression system of embodiment 42, wherein the synthetic cell surface receptor polypeptide is selected from any one of TVA-EGF polypeptide, H-EGF polypeptide, H-IGF1 polypeptide, and variants thereof.
44. The nucleic acid expression system of any one of embodiments 1-43, wherein the nucleic expression system comprises at least 2, 3, or 4 oligonucleotides each comprising a sequence that encodes a distinct helper polypeptide for enhancing expression of the oligonucleotide comprising a payload sequence in a target cell.
45. The nucleic acid expression system of embodiment 44, wherein the oligonucleotides each comprising a sequence that encodes a distinct helper polypeptide are each selected from the following: a nuclear localization signal (NLS) polypeptide, a DNA mimic polypeptide, a modulator of innate immunity, and a synthetic cell surface receptor polypeptide.
46. A nucleic acid expression system comprising:

(i) an oligonucleotide comprising a payload sequence, and

(ii) at least one oligonucleotide sequence comprising a sequence that encodes a helper polypeptide for enhancing nuclear import of the oligonucleotide comprising a payload sequence in a target cell.

47. The nucleic acid expression system of embodiment 46, wherein the helper polypeptide is or comprises a nuclear localization signal (NLS) polypeptide.
48. The nucleic acid expression system of embodiment 46 or 47, wherein the at least one oligonucleotide comprising a sequence that encodes a helper polypeptide is a synthetic oligonucleotide.
49. The nucleic acid expression system of any one of embodiments 46-48, wherein the at least one oligonucleotide comprising a sequence that encodes a helper polypeptide is a DNA oligonucleotide.
50. The nucleic acid expression system of any one of embodiments 46-48, wherein the at least one oligonucleotide comprising a sequence that encodes a helper polypeptide is an RNA oligonucleotide (e.g., a mRNA oligonucleotide).
51. The nucleic acid expression system of any one of embodiments 46-50, wherein the oligonucleotide comprising a payload sequence is a synthetic oligonucleotide.
52. The nucleic acid expression system of any one of embodiments 46-51, wherein the oligonucleotide comprising a payload sequence is a DNA oligonucleotide.
53. The nucleic acid expression system of any one of embodiments 46-51, wherein the oligonucleotide comprising a payload sequence is an RNA oligonucleotide (e.g., a mRNA oligonucleotide).
54. A nucleic acid expression system comprising:

(i) a synthetic DNA oligonucleotide comprising a payload sequence, and

(ii) at least one RNA oligonucleotide (e.g., a mRNA oligonucleotide) comprising a sequence that encodes a helper polypeptide for enhancing nuclear import of the oligonucleotide comprising a payload sequence in a target cell.

55. The nucleic acid expression system of embodiment 54, wherein the helper polypeptide is or comprises a nuclear localization signal (NLS) polypeptide.
56. The nucleic acid expression system of embodiment 47 or 55, wherein the NLS polypeptide is operatively connected to a DNA-binding domain (DBD) polypeptide.
57. A nucleic acid expression system comprising:

(i) an oligonucleotide comprising a payload sequence,

(ii) at least one oligonucleotide comprising a sequence that encodes a helper polypeptide, wherein the helper polypeptide comprises a nuclear localization signal (NLS) polypeptide, and

(iii) an oligonucleotide encoding a DNA-binding domain (DBD) polypeptide.

58. The nucleic acid expression system of embodiment 57, wherein oligonucleotide of (ii) and/or (iii) is an RNA oligonucleotide (e.g., a mRNA oligonucleotide).
59. The nucleic acid expression system of any one of embodiments 47-53 or 55-58, wherein the NLS polypeptide is an SV40 NLS polypeptide or variant thereof.
60. The nucleic acid expression system of any one of embodiments 47-53 or 55-58, wherein the NLS polypeptide is from EGL-13 polypeptide, c-Myc polypeptide, NLP polypeptide or TUS polypeptide.
61. The nucleic acid expression system of any one of embodiments 56-60, wherein the DBD polypeptide is not regulated by a small molecule.
62. The nucleic acid expression system of embodiment 61, wherein the DBD polypeptide is or comprises a Cro repressor polypeptide or a catalytically-inactive meganuclease variant.
63. The nucleic acid expression system of any one of embodiments 56-60, wherein the DBD polypeptide is a synthetic DBD polypeptide.
64. The nucleic acid expression system of embodiment 63, wherein the DBD polypeptide is or comprises a zinc finger polypeptide, a TAL domain polypeptide, or a catalytically-inactive Cas9 polypeptide.
65. The nucleic acid expression system of any one of embodiments 56-60, wherein the DBD polypeptide is a non-specific DBD polypeptide.
66. The nucleic acid expression system of embodiment 65, wherein the DBD polypeptide is or comprises Sso7d polypeptide, H-NS polypeptide, HU-1 polypeptide, HU-2 polypeptide, p6 polypeptide of 429, A104R polypeptide of ASFV, dsp polypeptide, TmHU polypeptide, HPhA polypeptide, or HCcp3 polypeptide.
67. The nucleic acid expression system of any one of embodiments 56-66, wherein the NLS polypeptide and DBD polypeptide forms a fusion polypeptide.
68. The nucleic acid expression system of any one of embodiments 56-66, wherein the NLS polypeptide and DBD polypeptide are separate polypeptides that can dimerize.
69. A nucleic acid expression system comprising:

(i) an oligonucleotide comprising a payload sequence, and

(ii) at least one oligonucleotide comprising a sequence that encodes a helper polypeptide for enhancing persistence or uptake of the oligonucleotide comprising a payload sequence in a target cell.

70. The nucleic acid expression system of embodiment 69, wherein the helper polypeptide is or comprises one or more of the following: a DNA mimic polypeptide, a modulator of innate immunity, and a synthetic cell surface receptor polypeptide.
71. The nucleic acid expression system of embodiment 69 or 70, wherein the at least one oligonucleotide comprising a sequence that encodes a helper polypeptide is a synthetic oligonucleotide.
72. The nucleic acid expression system of any one of embodiments 69-71, wherein the at least one oligonucleotide comprising a sequence that encodes a helper polypeptide is a DNA oligonucleotide.
73. The nucleic acid expression system of any one of embodiments 69-71, wherein the at least one oligonucleotide comprising a sequence that encodes a helper polypeptide is a RNA polypeptide (e.g., a mRNA oligonucleotide).
74. The nucleic acid expression system of any one of embodiments 69-73, wherein the oligonucleotide comprising a payload sequence is a synthetic oligonucleotide.
75. The nucleic acid expression system of any one of embodiments 69-74, wherein the oligonucleotide comprising a payload sequence is a DNA oligonucleotide.
76. The nucleic acid expression system of any one of embodiments 69-74, wherein the oligonucleotide comprising a payload sequence is an RNA oligonucleotide (e.g., a mRNA oligonucleotide).
77. A nucleic acid expression system comprising:

(i) a synthetic DNA oligonucleotide comprising a payload sequence, and

(ii) at least one RNA oligonucleotide (e.g., a mRNA oligonucleotide) comprising a sequence that encodes a helper polypeptide for enhancing persistence or uptake of the oligonucleotide comprising a payload sequence in a target cell.

78. The nucleic acid expression system of embodiment 77, wherein the helper polypeptide is or comprises one or more of the following: a DNA mimic polypeptide, a modulator of innate immunity, and a synthetic cell surface receptor polypeptide.
79. The nucleic acid expression system of any one of embodiments 69-78, wherein the helper polypeptide is or comprises a DNA mimic polypeptide.
80. The nucleic acid expression system of embodiment 79, wherein the DNA mimic polypeptide is selected from any one of Ocr polypeptide, ArdA polypeptide, NuiA polypeptide, HI1450 polypeptide, DMP12 polypeptide, MfpA polypeptide, Arn polypeptide, Gam polypeptide and variants thereof.
81. The nucleic acid expression system of any one of embodiments 69-78, wherein the helper polypeptide is or comprises a modulator of innate immunity.
82. The nucleic acid expression system of embodiment 81, wherein the modulator of innate immunity is selected from any one of vIRF1, ORF52/KicGAS, PLP2-TM, PLP2, US11, and variants thereof.
83. The nucleic acid expression system of any one of embodiments 69-78, wherein the helper polypeptide is or comprises a synthetic cell surface receptor polypeptide.
84. The nucleic acid expression system of embodiment 83, wherein the synthetic cell surface receptor polypeptide is selected from any one of TVA-EGF polypeptide, H-EGF polypeptide, H-IGF1 polypeptide and variants thereof.
85. The nucleic acid expression system of any one of embodiments 69-78, wherein the nucleic acid expression system comprises at least 2, 3, or 4 oligonucleotides each comprising a sequence that encodes a distinct helper polypeptide for enhancing persistence or uptake of the oligonucleotide comprising a payload sequence in a target cell.
86. The nucleic acid expression system of embodiment 85, wherein the helper polypeptide is selected from the following: a DNA mimic polypeptide, a modulator of innate immunity, and a synthetic cell surface receptor polypeptide.
87. The nucleic acid expression system of any one of embodiments 45-86, wherein the oligonucleotide comprising a payload sequence comprises homology arms.
88. The nucleic acid expression system of any one of embodiments 1-87, wherein the oligonucleotide comprising a payload sequence and the at least one oligonucleotide comprising a sequence that encodes a helper polypeptide or the composition that delivers a helper polypeptide are formulated for separate administration (e.g., in a sequential manner).
89. The nucleic acid expression system of any one of embodiments 1-87, wherein the oligonucleotide comprising a payload sequence and the at least one oligonucleotide comprising a sequence that encodes a helper polypeptide or the composition that delivers a helper polypeptide are formulated for concurrent administration.
90. The nucleic acid expression system of any one of embodiments 45-87, wherein the expression system further comprises an oligonucleotide comprising a sequence that encodes a targeted nuclease.
91. The nucleic acid expression system of embodiment 90, wherein the oligonucleotide comprising a sequence that encodes a targeted nuclease is a DNA oligonucleotide.
92. The nucleic acid expression system of embodiment 90, wherein the oligonucleotide comprising a sequence that encodes a targeted nuclease is an RNA oligonucleotide (e.g., a mRNA oligonucleotide).
93. The nucleic acid expression system of any one of embodiments 90-92, wherein the targeted nuclease is a zinc-finger nuclease (ZFN), TAL effector domain nuclease (TALEN), or an engineered CRISPR/Cas9 system.
94. The nucleic acid expression system of any one of embodiments 90-93, wherein the oligonucleotide comprising a payload sequence, the at least one oligonucleotide comprising a sequence that encodes a helper polypeptide or the composition that delivers a helper polypeptide, and/or the oligonucleotide comprising a sequence that encodes a targeted nuclease are formulated for separate administration (e.g., in a sequential manner).
95. The nucleic acid expression system of any one of embodiments 90-93, wherein the oligonucleotide comprising a payload sequence, the at least one oligonucleotide comprising a sequence that encodes a helper polypeptide or the composition that delivers a helper polypeptide, and/or the oligonucleotide comprising a sequence that encodes a targeted nuclease are formulated for concurrent administration.
96. A composition comprising the nucleic acid expression system of any one of embodiments 1-95.
97. The composition of embodiment 96, wherein the composition is a pharmaceutical composition.
98. A pharmaceutical composition comprising the nucleic acid expression system of any one of embodiments 1-95, and a pharmaceutically acceptable carrier.
99. A cell comprising the nucleic acid expression system of any one of embodiments 1-95.
100. A method of enhancing expression of an oligonucleotide comprising a payload sequence in a target cell, the method comprising:

contacting a target cell with an oligonucleotide comprising a payload sequence; and

contacting the target cell with at least one oligonucleotide comprising a sequence that encodes a helper polypeptide.

101. The method of embodiment 100, wherein the helper polypeptide is selected from the following: a nuclear localization signal (NLS) polypeptide, a DNA mimic polypeptide, a modulator of innate immunity, and a synthetic cell surface receptor polypeptide.
102. The method of embodiment 100 or 101, wherein the at least one oligonucleotide comprising a sequence that encodes a helper polypeptide is a synthetic oligonucleotide.
103. The method of any one of embodiments 100-102, wherein the at least one oligonucleotide comprising a sequence that encodes a helper polypeptide is a DNA oligonucleotide.
104. The method of any one of embodiments 100-102, wherein the at least one oligonucleotide comprising a sequence that encodes a helper polypeptide is an RNA oligonucleotide (e.g., a mRNA oligonucleotide).
105. The method of any one of embodiments 100-104, wherein the at least one oligonucleotide comprising a payload sequence is a synthetic oligonucleotide.
106. The method of any one of embodiments 100-105, wherein the at least one oligonucleotide comprising a payload sequence is a DNA oligonucleotide.
107. The method of any one of embodiments 100-105, wherein the at least one oligonucleotide e comprising a payload sequence is an RNA oligonucleotide (e.g., a mRNA oligonucleotide).
108. The method of any one of embodiments 100-107, wherein the target cell is contacted with the oligonucleotide comprising a payload sequence and the at least one oligonucleotide comprising a sequence that encodes a helper polypeptide separately (e.g., in a sequential manner).
109. The method of any one of embodiments 100-107, wherein the target cell is contacted with the oligonucleotide comprising a payload sequence and the at least one oligonucleotide comprising a sequence that encodes a helper polypeptide concurrently.
110. The method of any one of embodiments 100-107, wherein the oligonucleotide comprising a payload sequence and the at least one oligonucleotide comprising a sequence that encodes a helper polypeptide are part of a vector.
111. A method of enhancing expression of an oligonucleotide comprising a payload sequence in a target cell, the method comprising:

contacting a target cell with an oligonucleotide comprising a payload sequence; and

contacting the target cell with at least one RNA oligonucleotide (e.g., mRNA oligonucleotide) comprising a sequence that encodes a helper polypeptide.

112. The method of embodiment 111, wherein the helper polypeptide is or comprises one or more of the following: a nuclear localization signal (NLS) polypeptide, a DNA mimic polypeptide, a modulator of innate immunity, and a synthetic cell surface receptor polypeptide.
113. The method of any one of embodiments 100-112, wherein the oligonucleotide comprising a payload sequence comprises homology arms.
114. The method of any one of embodiments 100-113, further comprises contacting the target cell with an oligonucleotide comprising a sequence that encodes a targeted nuclease.
115. The method of embodiment 114, wherein the oligonucleotide comprising a sequence that encodes a targeted nuclease is a DNA oligonucleotide.
116. The method of embodiment 114, wherein the oligonucleotide comprising a sequence that encodes a targeted nuclease is an RNA oligonucleotide (e.g., a mRNA oligonucleotide).
117. The method of any one of embodiments 114-116, wherein the targeted nuclease is a zinc-finger nuclease (ZFN), TAL effector domain nuclease (TALEN), or an engineered CRISPR/Cas9 system.
118. A method of enhancing expression of an oligonucleotide comprising a payload sequence in a target cell, the method comprising:

contacting a target cell with an oligonucleotide comprising a payload sequence; and

contacting the target cell with a composition that delivers a helper polypeptide.

119. The method of embodiment 118, wherein the helper polypeptide is selected from the following: a nuclear localization signal (NLS) polypeptide, a DNA mimic polypeptide, a modulator of innate immunity, and a synthetic cell surface receptor polypeptide.
120. The method of embodiment 118 or 119, wherein the composition that delivers a helper polypeptide is or comprises (i) an oligonucleotide comprising a sequence that encodes a helper polypeptide and/or (ii) a helper polypeptide.
121. The method of any one of embodiments 100-120, wherein the helper polypeptide is or comprises a NLS polypeptide.
122. The method of embodiment 121, wherein the NLS polypeptide is an SV40 NLS polypeptide or variant thereof.
123. The method of embodiment 121, wherein the NLS polypeptide is an NLS domain from EGL-13 polypeptide, c-Myc polypeptide, NLP polypeptide or TUS polypeptide.
124. The method of any one of embodiments 121-123, further comprising contacting the target cell with an oligonucleotide comprising a sequence that encodes a DNA-binding domain (DBD) polypeptide.
125. The method of embodiment 124, wherein the DBD polypeptide is not regulated by a small molecule.
126. The method of embodiment 125, wherein the DBD polypeptide is or comprises a Cro repressor polypeptide or a catalytically-inactive meganuclease variant.
127. The method of embodiment 124, wherein the DBD polypeptide is a synthetic DBD polypeptide.
128. The method of embodiment 127, wherein the DBD polypeptide is or comprises a zinc finger polypeptide, a TAL domain polypeptide, or a catalytically-inactive Cas9 polypeptide.
129. The method of embodiment 124, wherein the DBD polypeptide is a non-specific DBD polypeptide.
130. The method of embodiment 129, wherein the DBD polypeptide is or comprises Sso7d polypeptide, H-NS polypeptide, HU-1 polypeptide, HU-2 polypeptide, p6 polypeptide of 429, A104R polypeptide of ASFV, dsp polypeptide, TmHU polypeptide, HPhA polypeptide, or HCcp3 polypeptide.
131. The method of any one of embodiments 124-130, wherein the NLS polypeptide and DBD polypeptide forms a fusion polypeptide.
132. The method of any one of embodiments 124-130, wherein the NLS polypeptide and DBD polypeptide are separate polypeptides that can dimerize.
133. The method of embodiment 132, wherein the NLS polypeptide and DBD polypeptide dimerize through inducible dimerization domains.
134. The method of embodiment 133, wherein the inducible dimerization domain is a rapamycin-inducible FRB/FKBP pair.
135. The method of any one of embodiments 100-120, wherein the helper polypeptide is or comprises a DNA mimic polypeptide.
136. The method of enhancing expression of an DNA oligonucleotide of embodiment 135, wherein the DNA mimic polypeptide is selected from any one of Ocr polypeptide, ArdA polypeptide, NuiA polypeptide, HI1450 polypeptide, DMP12 polypeptide, MfpA polypeptide, Arn polypeptide, Gam polypeptide and variants thereof.
137. The method of any one of embodiments 100-120, wherein the helper polypeptide is or comprises a modulator of innate immunity.
138. The method of embodiment 137, wherein the modulator of innate immunity is selected from any one of vIRF1 polypeptide, ORF52/KicGAS polypeptide, PLP2-TM polypeptide, PLP2 polypeptide, US11 polypeptide, and variants thereof.
139. The method of any one of embodiments 100-120, wherein the helper polypeptide is or comprises a synthetic cell surface receptor polypeptide.
140. The method of embodiment 139, wherein the synthetic cell surface receptor polypeptide is selected from any one of TVA-EGF polypeptide, H-EGF polypeptide, H-IGF1 polypeptide and variants thereof.
141. The method of any one of embodiments 100-140, wherein the target cell is contacted with at least 2, 3, or 4 oligonucleotides each comprising a sequence that encodes a distinct helper polypeptide.
142. The method of embodiment 141, wherein the helper polypeptide is selected from the following: a nuclear localization signal (NLS) polypeptide, a DNA mimic polypeptide, a modulator of innate immunity, and a synthetic cell surface receptor polypeptide.
143. A method of increasing nuclear localization of an oligonucleotide comprising a payload sequence in a target cell, the method comprising:

contacting a target cell with an oligonucleotide comprising a payload sequence; and

contacting the target cell with an oligonucleotide comprising a sequence that encodes a helper polypeptide for enhancing nuclear import of the oligonucleotide comprising a payload sequence in a target cell.

144. A method of increasing nuclear localization of an oligonucleotide comprising a payload sequence in a target cell, the method comprising:

contacting a target cell with a DNA oligonucleotide comprising a payload sequence; and contacting the target cell with an at least one RNA oligonucleotide (e.g., mRNA oligonucleotide) comprising a sequence that encodes a helper polypeptide for enhancing nuclear import of the oligonucleotide comprising a payload sequence in a target cell.

145. The method of embodiment 143 or 144, wherein the helper polypeptide is or comprises a nuclear localization signal (NLS) polypeptide.
146. The method of embodiment 145, wherein the NLS polypeptide is operatively connected to a DNA-binding domain (DBD) polypeptide.
147. A method comprising:

contacting a target cell with an oligonucleotide comprising a payload sequence;

contacting the target cell with at least one oligonucleotide comprising a sequence that encodes a helper polypeptide, wherein the helper polypeptide comprises a nuclear localization signal (NLS) polypeptide, and

contacting the target cell with an oligonucleotide comprising a sequence that encodes a DNA-binding domain (DBD) polypeptide.

148. The method of any one of embodiments 145-147, wherein the NLS polypeptide is an SV40 NLS polypeptide or variant thereof.
149. The method of any one of embodiments 145-147, wherein the NLS polypeptide is from EGL-13 polypeptide, c-Myc polypeptide, NLP polypeptide or TUS polypeptide.
150. The method of any one of embodiments 145-149, wherein the DBD polypeptide is not regulated by a small molecule.
151. The method of embodiment 150, wherein the DBD polypeptide is or comprises a Cro repressor polypeptide or a catalytically-inactive meganuclease variant.
152. The method of any one of embodiments 145-149, wherein the DBD polypeptide is a synthetic DBD polypeptide.
153. The method of embodiment 152, wherein the DBD polypeptide is or comprises a zinc finger polypeptide, a TAL domain polypeptide, or a catalytically-inactive Cas9 polypeptide.
154. The method of any one of embodiments 145-149, wherein the DBD polypeptide is a non-specific DBD polypeptide.
155. The method of embodiment 154, wherein the DBD polypeptide is or comprises Sso7d polypeptide, H-NS polypeptide, HU-1 polypeptide, HU-2 polypeptide, p6 polypeptide of 429, A104R polypeptide of ASFV, dsp polypeptide, TmHU polypeptide, HPhA polypeptide, or HCcp3 polypeptide.
156. The method of any one of embodiments 145-155, wherein the NLS polypeptide and DBD polypeptide form a fusion polypeptide.
157. The method of any one of embodiments 145-155, wherein the NLS polypeptide and DBD polypeptide are separate polypeptides that can dimerize.
158. The method of embodiment 157, wherein the NLS polypeptide and DBD polypeptide dimerize through inducible dimerization domains.
159. The method of embodiment 158, wherein the inducible dimerization domain is a rapamycin-inducible FRB/FKBP pair.
160. A method of enhancing persistence or uptake of an oligonucleotide comprising payload sequence in a target cell, the method comprising:

contacting a target cell with an oligonucleotide comprising a payload sequence; and

contacting the target cell with an oligonucleotide comprising a sequence that encodes a helper polypeptide for enhancing persistence or uptake of the oligonucleotide comprising a payload sequence in a target cell.

161. A method of enhancing persistence or uptake of an oligonucleotide comprising a payload sequence in a target cell, the method comprising:

contacting a target cell with a DNA oligonucleotide comprising a payload sequence; and

contacting the target cell with an at least one RNA oligonucleotide (e.g., mRNA oligonucleotide) comprising a sequence that encodes a helper polypeptide for enhancing persistence or uptake of the oligonucleotide comprising a payload sequence in a target cell.

162. The method of embodiment 160 or 161, wherein the helper polypeptide is or comprises one or more of the following: a DNA mimic polypeptide, a modulator of innate immunity, and a synthetic cell surface receptor polypeptide.
163. The method of any one of embodiments 160-162, wherein the helper polypeptide is or comprises a DNA mimic polypeptide.
164. The method of embodiment 163, wherein the DNA mimic polypeptide is selected from any one of Ocr polypeptide, ArdA polypeptide, NuiA polypeptide, HI1450 polypeptide, DMP12 polypeptide, MfpA polypeptide, Arn polypeptide, Gam polypeptide and variants thereof.
165. The method of any one of embodiments 160-162, wherein the helper polypeptide is or comprises a modulator of innate immunity.
166. The method of embodiment 165, wherein the modulator of innate immunity is selected from any one of vIRF1 polypeptide, ORF52/KicGAS polypeptide, PLP2-TM polypeptide, PLP2 polypeptide, US11 polypeptide, and variants thereof.
167. The method of any one of embodiments 160-162, wherein the helper polypeptide is or comprises a synthetic cell surface receptor polypeptide.
168. The method of embodiment 167, wherein the synthetic cell surface receptor polypeptide is selected from any one of TVA-EGF polypeptide, H-EGF polypeptide, H-IGF1 polypeptide, and variants thereof.
169. The method of any one of embodiments 160-169, wherein the target cell is contacted with at least 2, 3, or 4 oligonucleotides each comprising a sequence that encodes a distinct helper polypeptide for enhancing persistence or uptake of the oligonucleotide comprising a payload sequence in a target cell.
170. The method of embodiment 169, wherein the helper polypeptide is selected from the following: a DNA mimic polypeptide, a modulator of innate immunity, and a synthetic cell surface receptor polypeptide.
171. The method of any one of embodiments 100-170, wherein the target cell is contacted with the oligonucleotide comprising a payload sequence and the at least one oligonucleotide comprising a sequence that encodes a helper polypeptide or the composition that delivers a helper polypeptide separately (e.g., in a sequential manner).
172. The method of any one of embodiments 100-170, wherein the target cell is contacted with the oligonucleotide comprising a payload sequence and the at least one oligonucleotide comprising a sequence that encodes a helper polypeptide or the composition that delivers a helper polypeptide concurrently.
173. The method of any one of embodiments 118-170, wherein the oligonucleotide comprising a payload sequence comprises homology arms.
174. The method of any one of embodiments 118-170, further comprising contacting the target cell with an oligonucleotide comprising a sequence that encodes a targeted nuclease.
175. The method of embodiment 174, wherein the oligonucleotide comprising a sequence that encodes a targeted nuclease is a DNA oligonucleotide.
176. The method of embodiment 174, wherein the oligonucleotide comprising a sequence that encodes a targeted nuclease is an RNA oligonucleotide (e.g., mRNA oligonucleotide).
177. The method of any one of embodiments 174-176, wherein the targeted nuclease is a zinc-finger nuclease (ZFN), TAL effector domain nuclease (TALEN), or an engineered CRISPR/Cas9 system.
178. The method of any one of embodiments 114-117 or 174-177, wherein the target cell is contacted with the oligonucleotide comprising a payload sequence, the at least one oligonucleotide comprising a sequence that encodes a helper polypeptide or the composition that delivers a helper polypeptide, and/or the oligonucleotide comprising a sequence that encodes a targeted nuclease separately.
179. The method of any one of embodiments 114-117 or 174-177, wherein the target cell is contacted with the oligonucleotide comprising a payload sequence, the at least one oligonucleotide comprising a sequence that encodes a helper polypeptide or the composition that delivers a helper polypeptide, and/or the oligonucleotide comprising a sequence that encodes a targeted nuclease concurrently.
180. A method of enhancing expression of a non-viral oligonucleotide comprising a payload sequence in a subject, the method comprising: administering at least one oligonucleotide comprising a sequence that encodes a helper polypeptide to a subject that has been or is to be administered a non-viral oligonucleotide comprising a payload sequence, such that the subject receives both.
181. A method of enhancing expression of a non-viral oligonucleotide comprising a payload sequence in a subject, the method comprising: administering a non-viral oligonucleotide comprising a payload sequence to a subject that has been or is to be administered at least one oligonucleotide comprising a sequence that encodes a helper polypeptide, such that the subject receives both.
182. The method of embodiment 180 or 181 wherein the helper polypeptide is or comprises one or more of the following: a nuclear localization signal (NLS) polypeptide, a DNA mimic polypeptide, a modulator of innate immunity, and a synthetic cell surface receptor polypeptide.
183. The method of any one of embodiments 180-182, wherein the subject is administered at least 2, 3, or 4 oligonucleotides each comprising a sequence that encodes a distinct helper polypeptide.
184. The method of embodiment 183, wherein the helper polypeptide is selected from the following: a nuclear localization signal (NLS) polypeptide, a DNA mimic polypeptide, a modulator of innate immunity, and a synthetic cell surface receptor polypeptide.
185. A method of enhancing expression of a non-viral oligonucleotide comprising a payload sequence in a subject, the method comprising: administering at least one RNA oligonucleotide (e.g., mRNA oligonucleotide) comprising a sequence that encodes a helper polypeptide to a subject that has been or is to be administered a non-viral oligonucleotide comprising a payload sequence, such that the subject receives both.
186. A method of enhancing expression of a non-viral oligonucleotide in a subject, the method comprising: administering a non-viral oligonucleotide comprising a payload sequence to a subject that has been or is to be administered at least one RNA oligonucleotide (e.g., mRNA oligonucleotide) comprising a sequence that encodes a helper polypeptide, such that the subject receives both.
187. The method of embodiment 185 or 186, wherein the helper polypeptide is or comprises one or more of the following: a nuclear localization signal (NLS) polypeptide, a DNA mimic polypeptide, a modulator of innate immunity, and a synthetic cell surface receptor polypeptide.

Other features of the invention will become apparent in the course of the following descriptions of exemplary embodiments, which are given for illustration of the invention and are not intended to be limiting thereof.

### EXEMPLIFICATION

### Example 1—Transfection Efficiency with Co-Delivery of a Model Payload with an Oligonucleotide Comprising a Sequence that Encodes a Helper Polypeptide

The present Example describes construction of an exemplary helper polypeptide construct and a model payload oligonucleotide encoding a reporter and further demonstrates that co-transfection with an exemplary helper polypeptide construct can increase expression of a model payload. In particular, this experiment assessed expression of a model reporter linear DNA oligonucleotide when co-transfected with TetR, NLS-TetR and control DNA oligonucleotide constructs.

Preparation of Exemplary DNA Constructs

Model TetR and NLS-TetR DNA oligonucleotide constructs were synthesized using the following sequences:

TetR:
(SEQ ID NO: 3)
TAATACGACTCACTATAGGgCTAGCCCCGGGGATATCGCCACCatgTCTA
GATTAGATAAAAGTAAAGTGATTAACAGCGCATTAGAGCTGCTTAATGAG
GTCGGAATCGAAGGTTTAACAACCCGTAAACTCGCCCAGAAGCTAGGTGT
AGAGCAGCCTACATTGTATTGGCATGTAAAAAATAAGCGGGCTTTGCTCG
ACGCCTTAGCCATTGAGATGTTAGATAGGCACCATACTCACTTTTGCCCT
TTAGAAGGGGAAAGCTGGCAAGATTTTTTACGTAATAACGCTAAAAGTTT
TAGATGTGCTTTACTAAGTCATCGCGATGGAGCAAAAGTACATTTAGGTA
CACGGCCTACAGAAAAACAGTATGAAACTCTCGAAAATCAATTAGCCTTT
TTATGCCAACAAGGTTTTTCACTAGAGAATGCATTATATGCACTCAGCGC
TGTGGGGCATTTTACTTTAGGTTGCGTATTGGAAGATCAAGAGCATCAAG
TCGCTAAAGAAGAAAGGGAAACACCTACTACTGATAGTATGCCGCCATTA
TTACGACAAGCTATCGAATTATTTGATCACCAAGGTGCAGAGCCAGCCTT
CTTATTCGGCCTTGAATTGATCATATGCGGATTAGAAAAACAACTTAAAT
GTGAAAGTGGGTCCtaaTAGTTCTAGAGCGGCCGCTTCCCTTTAGTGAGG
GTTAATGCTTCGAG
NLS-TetR:
(SEQ ID NO: 4)
TAATACGACTCACTATAGGgCTAGCCCCGGGGATATCGCCACCatgCCAA
AAAAGAAGAGAAAGGTGgaagaccccggcggtggctctggaggtggtggg
tccggcggtggctctTCTAGATTAGATAAAAGTAAAGTGATTAACAGCGC
ATTAGAGCTGCTTAATGAGGTCGGAATCGAAGGTTTAACAACCCGTAAAC
TCGCCCAGAAGCTAGGTGTAGAGCAGCCTACATTGTATTGGCATGTAAAA
AATAAGCGGGCTTTGCTCGACGCCTTAGCCATTGAGATGTTAGATAGGCA
CCATACTCACTTTTGCCCTTTAGAAGGGGAAAGCTGGCAAGATTTTTTAC
GTAATAACGCTAAAAGTTTTAGATGTGCTTTACTAAGTCATCGCGATGGA
GCAAAAGTACATTTAGGTACACGGCCTACAGAAAAACAGTATGAAACTCT
CGAAAATCAATTAGCCTTTTTATGCCAACAAGGTTTTTCACTAGAGAATG
CATTATATGCACTCAGCGCTGTGGGGCATTTTACTTTAGGTTGCGTATTG
GAAGATCAAGAGCATCAAGTCGCTAAAGAAGAAAGGGAAACACCTACTAC
TGATAGTATGCCGCCATTATTACGACAAGCTATCGAATTATTTGATCACC
AAGGTGCAGAGCCAGCCTTCTTATTCGGCCTTGAATTGATCATATGCGGA
TTAGAAAAACAACTTAAATGTGAAAGTGGGTCCtaaTAGTTCTAGAGCGG
CCGCTTCCCTTTAGTGAGGGTTAATGCTTCGAG

Both constructs were PCR amplified using a commercially available polymerase (e.g., Herculase II polymerase (Agilent)) and the following primers:

TOPO_fwd
(TAATACGACTCACTATAGGGCTAG; SEQ ID NO: 5)
TOPO_rev
(CTCGAAGCATTAACCCTCAC; SEQ ID NO: 6)

Oligonucleotides encoding helper polypeptides can be cloned using any recombinant methods known in the art. Amplicons were each TOPO cloned into a pcDNA3.1D/V5-His-TOPO vector (Thermo Fisher) to produce pcDNA3.1-TetO and pcDNA-NLS-TetO plasmids. pcDNA3.1-TetR and pcDNA3.1-NLS-TetR plasmid DNA were prepared using standard kits, for example, the ZymoPURE-EndoZero Plasmid Maxiprep Kit and ZymoPURE Plasmid Miniprep Kit (Zymo Research). Control plasmid DNA, e.g., pUC19, can be purchased from commercial vendors such as New England Biolabs.

A model payload oligonucleotide was constructed that includes a tetracycline repressor recognition sequence (tetO) and a Gaussia luciferase reporter. To produce this exemplary Gaussia luciferase expressing DNA oligonucleotide, a luciferase gene was fused to a tetO site (TCCCTATCAGTGATAGAGA; SEQ ID NO: 7) by performing a PCR with pCMV-GLuc Control (New England Biolabs) as the template using the following primers:

GLuc_fwd
(AACAAGGCAAGGCTTGAC; SEQ ID NO: 8)
GLuc_tetO_rev
(TATTCACGGCGCACGAGCTGCGACTCTCTATCACTGATAGGGAA
GCATGCCTGCTATTGC; SEQ ID NO: 9).

PCR amplification was done using a commercially available polymerase (e.g., Herculase II polymerase). The GLuc-tetO amplicons can be cloned into an appropriate vector using any methods used in the art. For example, GLuc-tetO amplicons were TOPO cloned into pCR-XL-2-TOPO (Thermo Fisher) to produce a pCR-GLuc-tetO plasmid.

An array of 10 tetO repeats was constructed by synthesizing it as two half-arrays with the following sequences:

10x_tetO_L:
(SEQ ID NO: 10)
AGAGACCAATTGCATTCATTTTATGTTTCAGGTTCAGGGGGAGGTGTGG
GAGGTTTTTTAAAGCAAGTAAAACCTCTACAAATGTGGTAAAATCGATA
AGtccctatcagtgatagagaacggatctacgtcacaacgatccctatc
agtgatagagacggttttgtagaagcctaggtccctatcagtgatagag
atcaagcgaggtgatttcaactccctatcagtgatagagagcgtacaat
cccctaaagtatccctatcagtgatagagaACCACGAAGACAGGATTGT
CCGATCCCATATTACGACCTT
10x_tetO_R:
(SEQ ID NO: 11)
ACCACGAAGACAGGATTGTCCGATCCCATATTACGACCTTtccctatca
gtgatagagaccattagtcggcacaagtggtccctatcagtgatagaga
atgtgttgcgattgcccgcttccctatcagtgatagagagttgccgtac
gcgttgaacatccctatcagtgatagagatgcgtatagagcgggtcatt
tccctatcagtgatagagaGTCGACACACAATCTCCCAGCTCACT

These two fragments were fused to generate 10×_tetO. Exemplary reaction conditions can include the following for a 25 μL reactions: 100 ng 10×_tetO_L, a molar equivalent of 10×_tetO_R, 250 μM each dNTP, 1× commercially available polymerase and accompanying buffer (e.g., Herculase II), which can then be fused using the following exemplary thermocycling conditions: 95° C.-2 min, 20 cycles of (95° C.-15 sec, 72° C.-1 sec), reduce temperature to 50° C. at 0.1° C./sec, 50° C.-1 sec, 72° C.-20 sec, and 72° C.-3 min.

10×_tetO fusion products were further amplified using the following primers:

10x_tetO_fwd
(AGAGACCAATTGCATTCATTTTATG; SEQ ID NO: 12)
10x_tetO_rev
(AGTGAGCTGGGAGATTGTG; SEQ ID NO: 13)

10×_tetO can be cloned into an appropriate plasmid using any methods known in the art. For example, 10×_tetO was cloned into a PmeI-digested pCR-GLuc-tetO plasmid using a commercially available blunt end ligase (e.g., Blunt/TA Ligase Master Mix (New England Biolabs)) to produce a pCR-GLuc-11×TetO construct. The plasmid was then purified using a commercially available kit (e.g., ZymoPURE-EndoZero Plasmid Maxiprep Kit). Linear PCR product was amplified with GLuc_fwd and GLuc_tetO_rev primers using a commercially available polymerase (Herculase II polymerase). Resulting PCR product was purified using a commercially available clean-up kit (e.g., DNA Clean & Concentrator (Zymo Research)), digested with DpnI, then purified again using a commercially available clean-up kit (e.g., DNA Clean & Concentrator (Zymo Research)).

Cell Culture

Transfection efficiency and expression of oligonucleotides encoding a payload may be assessed using mouse muscle cells, for example, C2C12 myoblasts. C2C12 myoblasts (ATCC CRL-1772) can be maintained in high glucose DMEM (Thermo Fisher 12430) supplemented with 15% dialyzed FBS (Thermo Fisher). Cell cultures can be maintained in a 5% CO2, 37° C. humidified incubator, and cells can be passaged periodically (e.g., every 1-3 days using TrypLE Express (Thermo Fisher)).

Transfection

Transfection can be carried using methods known in the art. For example, C2C12 cells were plated to 24-well plates at a density 20,000 cells/well in an appropriate media (e.g., high glucose DMEM supplemented with 15% Tet System Approved FBS (Clontech)). Cells were transfected 1 day after plating. Transfection was carried out using Lipofectamine 3000 (Thermo Fisher). 200 ng linear GLuc-11tetO DNA and 500 ng of pcDNA3.1-TetO, pcDNA3.1-NLS-TetO, or pUC19 were mixed with 1.05 uL Lipofectamine 3000, incubated at room temperature for approximately 15 min., and added to the cultures.

Luciferase Assay

Luciferase luminescence can be assessed using standard protocols. For example, cell culture media was collected at approximately 20 h. following transfection and the concentration of a secreted luciferase reporter was assayed using a commercially available luciferase assay kit (e.g., BioLux Gaussia Luciferase Assay Kit (NEB)), using a stabilized assay format with a 1:10 substrate:stabilizer ratio. Luminescence was measured using a plate reader (e.g., EnVision (PerkinElmer)).

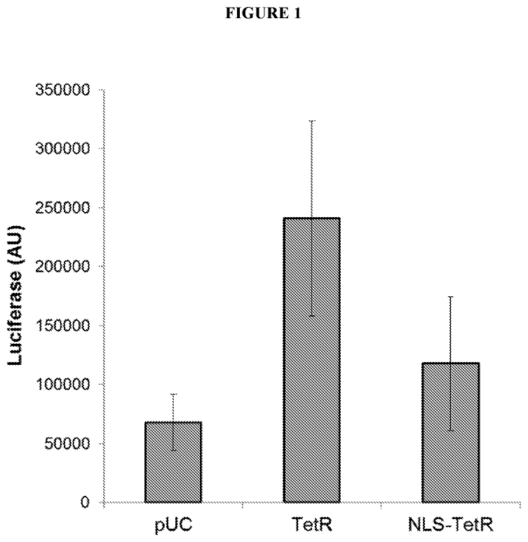

Enhancement of transfection efficiency of an exemplary linear DNA was observed by co-transfection with TetR and also with NLS-TetR (albeit to a lesser extent), see FIG. 1. The enhancement observed by co-transfection with TetR may be in part due to the presence of a cryptic NLS, perhaps within the cationic DNA-binding domain. For example, it has been reported that TetR can be used to regulate the expression of genomically-integrated reporters without an NLS fusion and fusion to an NLS may interfere with proper functioning of a TetR protein. Additionally, we have also observed that TetR and NLS-TetR may have different toxicities in C2C12 cultures. Consequently, there may be slight differences in timing of when the luciferase measurements are taken following transfection, which may affect relative performance of these two constructs. Lastly, different commercially available plasmid preparation kits were used in this experiment. Notably, controlling for preparation methods can reverse the relative enhancement observed with the TetR and NLS-TetR, for example, as is described in Example 2 below.

### Example 2—Transfection Efficiency with Sequential Delivery of a Model Construct Comprising a Sequence that Encodes a Helper Polypeptide and a Model Payload Oligonucleotide

The present Example describes expression of a model payload oligonucleotide when this model payload is separately delivered following delivery of a model oligonucleotide comprising a sequence that encodes a helper polypeptide. Specifically, this example describes expression of a model reporter linear DNA oligonucleotide that was transfected to cells that have already been transfected with an exemplary helper polypeptide construct. In particular, cells were first transfected with TetR, NLS-TetR, TmHU, or no-plasmid or pUC19 controls DNA oligonucleotide constructs, and then subsequently transfected with a model reporter linear DNA oligonucleotide.

Preparation of Exemplary DNA Constructs

Construction of pcDNA3.1-TetR and pcDNA3.1-NLS-TetR was as described in Example 1. A model construct that includes TmHA, a HU from Thermotoga maritima, was cloned into vector to generate a pcDNA3.1-TmHU DNA oligonucleotide construct. pcDNA3.1-TmHU was synthesized using the following sequence:

TmHU:
(SEQ ID NO: 14)
CACCATGACTAAAAAAGAGTTGATAGATCGGGTAGCAAAAAAGGCCGG
AGCAAAGAAAAAAGACGTAAAACTGATATTGGATACAATCCTGGAGAC
AATAACAGAAGCACTCGCCAAGGGCGAGAAAGTTCAGATCGTTGGATT
CGGTTCATTTGAAGTGCGAAAAGCCGCAGCAAGAAAGGGAGTGAACCC
ACAGACCCGAAAGCCAATCACTATTCCTGAAAGGAAAGTCCCCAAATT
CAAGCCCGGTAAGGCCCTCAAGGAAAAAGTTAAATGATAA

Oligonucleotides encoding helper polypeptides can be cloned using any recombinant methods known in the art. For example, TmHU DNA was TOPO cloned into a pcDNA3.1D/V5-His-TOPO vector, using standard methods. pcDNA3.1-TetR, pcDNA3.1-NLS-TetR, pcDNA3.1-TmHU, and pUC19 plasmid DNA were each prepared using a commercially available kit (e.g., QIAprep Miniprep kit (Qiagen)).

A model payload oligonucleotide that includes a tetracycline repressor recognition sequence (tetO) and a Gaussia luciferase reporter (pCR-GLuc-11×TetO PCR product) was prepared as described in Example 1.

Cell Culture

Cell culture was carried out as described in Example 1.

Transfection and Luciferase Assays

Transfection can be carried using methods known in the art. For example, C2C12 cells were plated to 24-well plates at a density 20,000 cells/well in an appropriate media (e.g., high glucose DMEM supplemented with 15% Tet System Approved FBS (Clontech)). Cells were transfected 1 day after plating. Transfection was carried out using 1 g of pcDNA3.1-TetR, pcDNA3.1-NLS-TetR, pcDNA3.1-TmHU, or pUC19 were mixed with 1.5 μL Lipofectamine 3000 and 2 μL P3000 in a 25 μL mixture. DNA-Lipofectamine complexes were incubated for about 15 min at room temperature, and the resulting mixture was added to each well of a 24-well plate of cultures. 5 h following plasmid transfection, 3 μg pCR-GLuc-11×TetO amplicons was added directly to each culture. Media was collected for analysis via the Gaussia luciferase assay at 16 h, 40 h, 111 h, 163 h, and 231 h. Cells were split at a 1:3 dilution using TrypLE Express at 16 h and 40 h.

Luciferase assays were carried out as described in Example 1.

NLS-TetR can enhance transfection efficiency, e.g., by facilitating nuclear uptake of the reporter DNA.

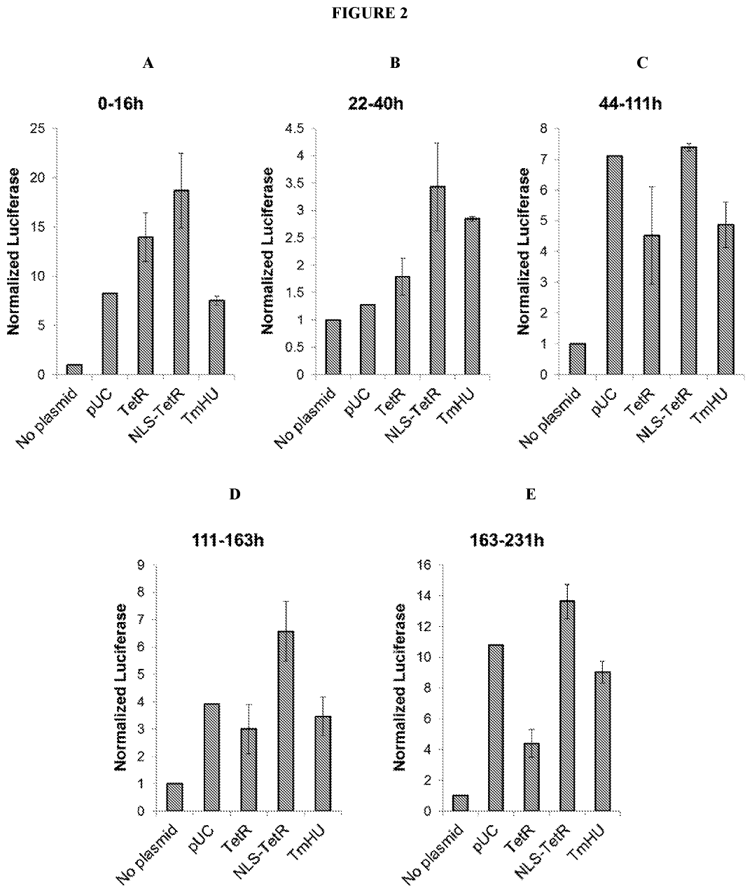

Results from the luciferase assay for samples at 16 h, 40 h, 111 h, 163 h, and 231 h are shown in FIG. 2, panels A-E, respectively.

Cells transfected with an NLS-TetR exhibited higher levels of expression than those transfected with plasmid control (pUC19) across all time points, demonstrating that prior expression of an NLS-DBD helper polypeptide enhanced expression of model payload oligonucleotide. Moreover, this enhancement was a prolonged effect, over the time assayed. However, we note that at 44 h-111 h, enhancement of expression was increased (relative to no plasmid, TetR and TmHU cells), but this increase was not significantly higher than that observed with control plasmid. Without wishing to be bound by theory, one potential explanation for this modest relative increase may be related to expression kinetics. Notably the 44 h-111 h time period approximately when peak expression is typically observed with a regularly transfected reporter plasmid. During peak reporter production transcriptional and translational machineries of cells may become saturated, such that a correlation between nuclear DNA copy number and protein expression levels no longer holds.

It was observed that TetR provides initial enhancement in efficiency and/or expression of a model payload oligonucleotide, but this effect decays over time. Notably, at later time points, a payload oligonucleotide expressed at a lower level in cells transfected with TetR than those transfected with control plasmid (pUC19). Without wishing to be bound by theory, one possible explanation for this decay in expression is that intrinsic NLS activity of a TetR polypeptide is counter-acted by its toxicity.

TmHA was observed to provide a transient enhancement of expression of a model payload oligonucleotide. Without wishing to be bound by theory, it is envisioned that this effect is mediated though cryptic NLS functionality. Further analysis may be conducted using NLS-fused variants of TmHA.

Further, it was observed that prior transfection with plasmid control DNA resulted in a substantial (5-10×) enhancement in transfection efficiency and/or expression of a model payload oligonucleotide at the earliest time-point. Without wishing to be bound by theory, this may be due to a DNA saturation of cytoplasmic nucleases, thereby prolonging survival of a model payload oligonucleotide. This supports a potential benefit of using polypeptides that structurally mimic DNA in order to overcome various cytoplasmic defenses.

### Example 3—DNA Mimics as Helper Polypeptides

The present Example describes screening and characterization of polypeptides that structurally mimic DNA for the ability to enhance transfection efficiency of a model payload oligonucleotide. Specifically, this example demonstrates that co-transfection of a model payload oligonucleotide with exemplary oligonucleotides encoding DNA mimic polypeptides can increase expression of the model payload. In particular, an initial screen identified two proteins (GenBank EKC62359.1 and EKC78842.1) as DNA mimic polypeptide candidates.

DNA Constructs

Sequences encoding candidate DNA mimic polypeptides were cloned into appropriate vectors using standard techniques. Exemplary vectors pcDNA3.1-EKC62359 and pcDNA3.1-EKC78842, which include the EKC62359.1 and EKC78842.1 sequences, respectively, were synthesized using the following sequences:

EKC62359:
(SEQ ID NO: 15)
CACCATGGTATCACAACTCTACGGAATCTATAGGCCCCAGCGCCCAGA
CACACTCCTTTCTGGCGCAGACGGTGAAAGTCTCGCACGGTACCTCGT
CCAGGAGGTGCAGCTTTTCGGAGAAGTGCATCCCGACCTGCTGAACCA
CATCGACTACGCTGCAATCGGGAGGGAGCTGGAGACTTCAGAAAATTA
TCTCTTCACTGATAATGGCATTTTCTATTACCGGTAGTGATAA
EKC78842:
(SEQ ID NO: 16)
CACCATGAGTCAGGACGAATACGAGCGATTCCAGGCCGCCATGGAAAT
CGGTGATCACACAGGGAGCATACAAGAGTTGATCAATCTTACCGAAAA
TTTGGATTGTTACGACGTGTATCCTGACATCCATGACCATGATGATCT
TGGAAGGTATTATATAGAAGAGCTGGATGCAATGCAAGTTCCCGAACA
TCTGAGGAATTACATAGACTATGAAGCATATGGCCGGGACATAGCCTT
GGAAGAGTCTGGGCAGTTCACTGATTTGGGTTATGTGAGGGACACAGG
CGATTCCTTTCACGAGTACTATGATGGAGAACGCGGTAGTATTCCAGA
GGAATACAGAGTGATGACTTTCCAAGATGATATTCCTGAAGAAGAGAT
ATCCGAATGGGCAATGGATCTCGCTTATGACATGGATGAATTTTTCAG
ACAAAACGACCCTCAATACGCCGCAGAACACCCAGAGGAACATGCCGC
TAAGGAAGAAATATATGAAAACCTGATGGCAGGGCGGATTAGTGCTTT
GGATGAGAAGTTGGCCGCTCTTGGGTAGTGATAA

Any recombinant methods known in the art can be used for preparation of DNA mimic vectors. For example, synthesized DNA was TOPO cloned into pcDNA3.1D/V5-His-TOPO vector. Resulting plasmids pcDNA3.1-EKC62359 and pcDNA3.1-EKC78842, as well as pUC19 control plasmid were prepared using a commercially available kit (e.g., QIAprep Miniprep kit).

pCR-GLuc-11×TetO PCR product was prepared as described in Example 1.

Cell Culture

Cell culture was carried out as described in Example 1.

Transfection and Luciferase Assays

Transfection can be carried using methods known in the art. For example, C2C12 cells were plated to 24-well plates at a density 20,000 cells/well in an appropriate media (e.g., high glucose DMEM supplemented with 15% Tet System Approved FBS (Clontech)). Cells were transfected 1 day after plating. Exemplary transfection reactions include 50 ng of pcDNA3.1-EKC62359, pcDNA3.1-EKC78842, or pCU19 were mixed with 0.075 μL Lipofectamine 3000 and 0.1 μL P3000 in a 50 μL mixture. The mixture was incubated for approximately 15 min at room temperature, and the resulting mixture was added to each well of a 24-well plate of cultures. 4 h following plasmid transfection, 3 μg pCR-GLuc-11×TetO amplicons was added directly to each culture. Media was collected for analysis 18 h after transfection of the reporter.

Luciferase assays were carried out as described in Example 1.

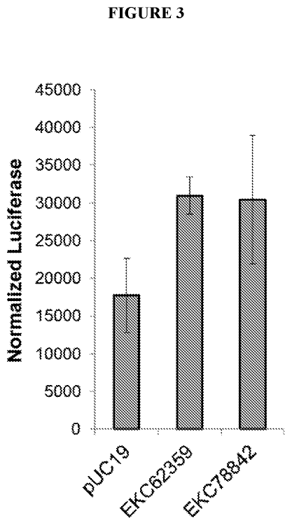

Results are shown in FIG. 3. Each of the two candidate DNA mimic polypeptides, GenBank EKC62359.1 and EKC78842.1, showed modest enhancement of the expression levels of a transfected luciferase reporter. These results show that oligonucleotides encoding a DNA mimic polypeptide can enhance expression of a model payload oligonucleotide. Without wishing to be bound by theory, DNA mimic polypeptides may enhance transfection efficiency by saturating the activity of cellular nucleases.

Both of these proteins are homologs of ArdA anti-restriction protein. These sequences were initially identified in human gut metagenome sequencing data. Other helper polypeptide candidates include proteins related to EKC62359 and EKC78842, as well as other families of DNA mimics.

Additional DNA mimic polypeptides can be screened for effectiveness to increases expression of a payload polynucleotide using, for example, a reporter assay as described herein. Polypeptides of interest may have DNA mimic properties suitable to function as a helper polypeptide. Some exemplary polypeptides that are candidates to act as DNA mimics include the following:

Ocr:
(SEQ ID NO: 17)
ATGGCAATGAGCAATATGACATATAACAATGTTTTCGACCACGCTTAT
GAGATGCTCAAGGAAAACATCAGATATGACGACATACGCGACACAGAT
GACCTGCACGACGCAATTCATATGGCAGCCGATAATGCCGTCCCTCAT
TACTATGCAGACATTTTCTCAGTTATGGCATCCGAGGGTATTGATCTG
GAGTTTGAGGACTCAGGCCTTATGCCAGACACTAAGGATGTCATACGG
ATCTTGCAAGCCCGGATCTACGAGCAGCTTACTATAGACCTCTGGGAG
GACGCAGAGGACCTCCTGAACGAGTATCTGGAGGAAGTCGAAGAGTAC
GAAGAAGACGAGGAATAG
ArdA:
(SEQ ID NO: 18)
ATGGATGACATGCAAGTTTACATTGCAAACTTGGGGAAGTACAATGAG
GGGGAACTGGTGGGTGCATGGTTCACCTTCCCAATCGACTTCGAGGAA
GTAAAAGAGAAAATCGGACTTAATGACGAGTACGAAGAGTATGCAATC
CACGATTACGAGCTGCCATTTACCGTCGACGAATACACTAGCATCGGA
GAACTTAATAGGCTTTGGGAAATGGTTTCCGAGTTGCCCGAAGAACTC
CAGTCAGAACTTTCCGCACTTCTTACCCACTTCAGCAGTATAGAAGAA
CTGTCAGAACACCAAGAAGACATCATAATACATAGTGATTGCGATGAT
ATGTACGATGTGGCCAGGTACTACATCGAAGAGACTGGGGCTTTGGGT
GAGGTCCCCGCTAGTCTCCAAAATTATATAGATTACCAAGCCTACGGC
CGCGATCTTGACCTGTCAGGGACTTTTATTTCTACTAACCACGGAATC
TTCGAGATCGTTTACTAG
NuiA:
(SEQ ID NO: 19)
ATGACTAAAACCAACTCCGAAATATTGGAGCAACTGAAGCAGGCTAGC
GACGGACTTCTGTTTATGTCAGAGTCCGAATACCCCTTTGAAGTATTC
CTGTGGGAGGGGTCCGCTCCACCTGTCACTCACGAGATCGTCTTGCAG
CAGACCGGACACGGTCAGGACGCACCATTCAAAGTTGTGGACATCGAC
TCCTTTTTTAGCCGAGCAACAACACCCCAAGATTGGTACGAAGACGAA
GAGAATGCTGTTGTGGCAAAGTTTCAGAAATTGCTCGAAGTCATCAAG
AGTAACCTTAAAAATCCCCAGGTGTATCGATTGGGAGAAGTGGAGCTG
GATGTGTACGTCATAGGGGAAACACCCGCCGGTAACCTGGCCGGGATC
TCTACTAAGGTCGTTGAAACATAG
HI1450:
(SEQ ID NO: 20)
ATGACAACCGAGATTAAGAAACTGGACCCAGATACAGCAATCGACATA
GCTTACGATATTTTCCTTGAGATGGCCGGTGAAAACCTCGATCCCGCT
GACATACTTCTGTTCAACCTCCAGTTTGAAGAGAGGGGGGGCGTAGAA
TTTGTCGAAACCGCAGATGACTGGGAGGAGGAGATTGGAGTCTTGATC
GACCCTGAAGAGTACGCCGAGGTGTGGGTTGGCCTGGTCAACGAGCAA
GACGAGATGGACGACGTCTTTGCTAAATTTCTTATCTCACACCGAGAG
GAGGACCGAGAGTTCCATGTCATTTGGAAGAAATAG
DMPl2
(SEQ ID NO: 21)
ATGAACGAGCACAACCTTTTGATCTTCTGCCTGAAGGACAATGTCTCT
ATAAGTGAGTACACAGAGATGATTGATTGGGCTTACAAGAATATCCAG
TCCGAAACTGTTGTAGAAATAACCGAGAACCAGATTATAGAATATCAG
AACCGGGGGTTGTGGAGACTCGTCTCTGAAATTACTGACAACTGGCTG
TTCGGTCCCAGTGAAGGGGATTGGCTTATAGACAAGGAATCTATACTT
GCTGTCAAAGAAAAGTTGCAGAACTCCGACTTCTCAACCGAGCCTCTT
GTCAAAAACATAATCCACGTGTTGGAATACGCTATCAAGAATGAGAAG
ACCGTTATCTTTCACTTTTAG
MfpA:
(SEQ ID NO: 22)
ATGCAGCAATGGGTTGATTGCGAGTTTACAGGACGGGATTTTAGGGAT
GAAGACTTGTCTAGGCTGCATACTGAGAGGGCCATGTTCAGCGAGTGC
GATTTTTCCGGCGTGAATCTGGCTGAAAGTCAGCATCGGGGAAGTGCA
TTTCGCAATTGCACCTTTGAGCGAACAACCCTTTGGCATTCAACTTTT
GCTCAATGTAGCATGCTGGGTAGTGTGTTCGTAGCATGTCGACTCAGA
CCCCTCACTCTCGACGATGTCGACTTCACCTTGGCCGTGCTTGGGGGG
AATGACCTCCGGGGGTTGAACTTGACTGGTTGCCGATTGCGGGAAACA
TCTTTGGTTGACACTGATCTCCGAAAATGTGTTCTGCGCGGGGCTGAC
CTCTCCGGCGCTCGGACTACAGGTGCAAGGTTGGACGACGCTGACTTG
AGGGGTGCTACCGTGGACCCAGTGCTTTGGCGAACTGCATCCCTTGTG
GGAGCACGGGTCGATGTCGACCAAGCCGTAGCTTTTGCAGCAGCCCAC
GGACTGTGTTTGGCCGGAGGCTAG
Arn:
(SEQ ID NO: 23)
ATGATTATAGACTCCCAGAGCGTTGTCCAATACACTTTTAAGATAGAC
ATCCTCGAGAAGCTCTATAAATTTTTGCCCAACCTTTACCATTCAATC
GTCAATGAGCTGGTCGAAGAACTTCATCTGGAGAACAACGACTTCCTG
ATAGGGACATATAAAGACCTTAGTAAAGCAGGTTATTTTTACGTCATA
CCAGCACCCGGCAAGAATATCGACGATGTGTTGAAGACAATAATGATT
TACGTCCACGATTACGAAATTGAGGATTATTTTGAGTAG
Gam:
(SEQ ID NO: 24)
ATGGACATAAATACTGAGACTGAGATAAAGCAGAAACATTCACTCACA
CCCTTTCCCGTTTTCCTCATAAGTCCAGCTTTCCGGGGGAGGTATTTT
CACTCCTACTTCCGCTCCAGTGCAATGAACGCTTATTACATCCAAGAC
CGACTGGAAGCCCAAAGCTGGGCCCGGCACTATCAGCAACTCGCTCGG
GAAGAGAAAGAAGCAGAGCTTGCCGATGATATGGAAAAAGGTTTGCCA
CAACACTTGTTCGAGTCCCTGTGCATAGACCATTTGCAACGGCATGGT
GCATCAAAAAAATCTATTACCCGCGCCTTTGACGATGATGTAGAGTTT
CAAGAAAGGATGGCAGAGCACATTAGGTACATGGTAGAGACCATTGCT
CACCATCAAGTGGATATAGACTCCGAGGTGTAG

Polypeptides of particular interest as DNA mimic polypeptides include metagenomic homologs of Ocr and ArdA, such as the following:

EKC78327:
(SEQ ID NO: 25)
ATGATAGATGACATGGCAGTATACATTGCAAATTTGGGTAAGTATAAC
GAAGGCTATTTGGTGGGTGCCTGGTTTACCTTCCCCATTGACGAAGAA
GATGTTAAAGAAAAGATAGGACTCAACGAACAGTACGAAGAGTATGCA
ATCCATGATACTGATAACTTCCCCATTGCAATAGGTGAGTATGTTAGC
ATAGAGGAACTCAACGAAATGTACGAAATGATTGAGGAACTGCCCGAC
TATATTGTCGAATGTCTCGATGAGTTTATTTCACACTACGGGACCTTG
GAGGAAGTCGTCGAACACAAAGACGATATTTACTACTATCCAGATTGT
GAGACAATGACTGACGTAGCCTGTTACTACATAGATGAGTTGCAAGCA
TTGGGCGACATACCACCTAGTCTCCAAAACTACATCGACTATGAAGCA
TATGGAAGAGATTTGGACATGGGCGGGTGTTTCATCGAGACAAGCCGA
GGGATGTGCGAAATTCCATATTAG
KKN72305:
(SEQ ID NO: 26)
ATGAAGTCAGACTTGCAAGAGATTCTCAATGATGCATTGGACGAATTG
AAGGAGCGAATGAAGGATTATCCCGATGAGGACGCTGATGACGTTGTA
AGCGAGATAGCAGACAGTAGCGTTCCAGTCTACTACTCTGACCTCTTG
AAGCTGGCTTCCGGCTGCAATGACCTTGCTACCGCTGAACCAGAATGC
GGACCAGCATTCGACGGGAAGCCAACCCCTGTGAATATTATTGCAGCT
AATGTTTACGAAGCAGTCGATCAACATCTCCGAAATTACTTGTCAGCC
ATTTAG
KKK84065:
(SEQ ID NO: 27)
ATGAGTCAAAGTTTGTACGAGATTATTAAACTTGCTAGGGAAGAGCTT
CGGGGACGAGCAAAGGATAACAAAGATGAGACCGAGCCCCACGACTCT
ATCCACGAGATTGCTGATTCATCTGTGCCAGTCTATACAGGCGATCTC
TTGCAGTTGGCAGCAGACAACTTGGAGCTGGCCACAGCTAAACCAGAG
CTTGGACCCGCCTTTGATGGCAGTCCCACACCAGTCAACATCGTGGCT
GCTAATGTATTTGAAGCCATTGAAGCTGGGCTGTGGGAAGAATGGAAG
GAAATCGAGTCAGAACGCGAAGATGCAGAGTTGGAAGAAACTGGCTAG
KKK64782:
(SEQ ID NO: 28)
ATGACTAGTATAGCTCGCCCCGATATAGTGGATCGCGTTTTGGCCGCC
GCAGCAGACAGGGCCAGGGAACTCGTCGCCGAGGAAAGGCGCCTCATT
GCAGAAGACGCTATTGATGTAGAGGTGGTCGTACGCACTGATAGATCC
AGTGGCGATGTTGTAACCTCTCGCAAGGGACGCTCCTCCTTTGCCACC
GAAGAGCCAGTTTTGTTGGATGAGTCACCAAGCGCAAAACATAGTGCA
GTGCGACCTAAGGGTGATGATATGTCTGACGAGAAGCGAACAACCCTC
TACGGGCTGGAGCGCGGAGTACGAGATGAAGTTAGAGAGCGCAGCAAG
GAACTTCTGGAGGACGTGTGCCCAGAAGACACCCTGACCGAGATCGCT
GATGGGTGGGTACCAATCTACACTTACAACATACTCCAGGTCGCTGCA
GACAACATGGACATGGCAACCCTGGAGCCTGAACTGGGACCCGCATTC
GATGGCACACCTACCCCTATCAACATAATTGCCGCCAACATATATAAG
GCACTCAATGCCGCAGCCTTCAAAGAATGGGCTAAAGTTCATCCCAAA
TGGCGCAAAAAGCTGGCCGGGAACGATTAG

### Example 4—Transfection Efficiency with Co-Delivery of a Model DNA Payload with an mRNA Comprising a Sequence that Encodes a Helper Polypeptide

The present Example describes co-transfection of a model payload oligonucleotide with a mRNA comprising a sequence that encodes a helper polypeptide construct. mRNA vectors may have a number of advantages in vivo, including lack of long-term helper protein expression and higher initial expression levels.

Examples 1-3 show use of exemplary helper polypeptides encoded by a DNA oligonucleotide. The same methods and materials as described in Examples 1-3 can be used to characterize sequential and co-delivery of a model payload with mRNA oligonucleotides encoding helper polypeptides. Specifically, mRNA oligonucleotides that encode TetR, NLS-TetR, TmHU, NLS-TmHU, and TmHU-NLS are prepared using a commercially available kit, such as the MegaScript T7 system (Thermo Fisher). mRNA products can be capped via the addition of 3′-O-Me-m7G(5′)pppp(5′)G (ARCA) to the synthesis reaction. Poly-adenylated tails can be introduced by adding a 120 T bases to the 3′ of the DNA template. Conditions can be optimized using natural non-standard nucleotides, including 5-methylcytidine and pseudouridine, to reduce the immunogenicity of the mRNA.

Experimental methods for characterization with model payloads encoding a luciferase reporter can be carried out largely as describe in Example 1 for DNA constructs. A model payload DNA oligonucleotide can be transfected into cells either as naked DNA or using Lipofectamine 3000. For example, mRNA can be transfected using Lipofectamine MessengerMAX, or co-packaged with the DNA in Lipofectamine 3000 liposomes.

mRNA oligonucleotides encoding helper polypeptides may provide improved timing, duration and potentially other properties to enhance transfection and/or expression of a payload oligonucleotide.

### Example 5—Non-Specific DNA Binders Fused to NLS Domains as Helper Polypeptides

This Example describes construction and characterization of non-specific DNA binding polypeptides fused to an NLS domain. The data provided herein indicate that NLS-TetR and a model DNA vector may be interacting in a non-specific manner. This suggests a DNA binding domain which largely or exclusively via non-specific interactions may also function. In addition to TmHU described above, additional DNA binding polypeptides were characterized, both as native proteins, and fused to a SV40 NLS domain:

Exemplary vectors with DNA binding polypeptides can be synthesized using the following sequences:

HPhA:
(SEQ ID NO: 29)
ATGTGGATGATGGGGGAGCTGCCTATTGCCCCTGTTGACCGATTGATA
AGAAAGGCCGGAGCAGAGAGGGTGTCTGAACAGGCCGCAAAGGTCCTC
GCCGAATATCTCGAGGAGTATGCAATAGAGATCGCCAAAAAAGCAGTA
GAGTTCGCTAGACATGCAGGTCGCAAAACCGTTAAAGTAGAAGACATC
AAATTGGCTATTAAGTCCTGA
HCcp3:
(SEQ ID NO: 30)
ATGGCCCCCAAAATGAAGGCCGCTATGAAAGCTAAAGCAATGAAGGCA
CGGTCAGTAGCCATGAGTAAGGGCGCTCTTTGTCAAGCAATAGCCGAT
GCTACAGAGAATAAGAAGAGTGCCATTGTTAAATTTATGGATGCCCTT
GCCGAGGTAGTTACTGCTGAGGTCAAAAAGACCGGGAAAATGACAATA
CCTGGGGTCACAATGATTAAAACCAGAAAAAAACCTGCAACAAAAGCA
GGGAAGCGGGAAATGTTTGGAAAGGTGGTGCTGGTAAAGGCCCAACCT
GCCAAGACAGTTGTGAAAGCCTTTCCCGTTAAAGCCTTGAAGACAGAC
TTTTGA
HU-2:
(SEQ ID NO: 31)
ATGAATAAAACACAGCTTATAGATGTAATCGCCGAAAAAGCAGAGCTT
TCAAAGACACAGGCTAAAGCAGCTCTGGAAAGCACTCTTGCTGCTATT
ACCGAGAGCCTCAAGGAAGGAGATGCAGTTCAACTGGTAGGATTTGGG
ACCTTTAAGGTCAATCATAGAGCCGAAAGAACCGGACGCAACCCACAG
ACTGGTAAAGAGATAAAAATTGCTGCCGCAAACGTACCTGCATTCGTA
TCCGGGAAAGCCCTTAAGGATGCTGTCAAGTGA
H10_C-term:
(SEQ ID NO: 32)
ATGGACGAACCCAAGAAATCAGTGGCCTTCAAAAAGACCAAGAAGGAA
ATCAAGAAGGTAGCCACGCCAAAAAAGGCATCCAAGCCCAAGAAGGCT
GCCTCCAAAGCCCCAACCAAGAAACCCAAAGCCACCCCGGTCAAAAAG
GCCAAGAAAAAGCTGGCTGCCACGCCCAAGAAAGCTAAAAAACCCAAG
ACTGTCAAAGCCAAGCCGGTCAAGGCATCCAAGCCCAAAAAGGCCAAA
CCAGTGAAACCCAAAGCAAAGTCCAGTGCCAAGAGGGCCGGCAAAAAG
AAATAA

DNA binding polypeptides of interest can be cloned into appropriate vectors using any recombinant methods known in the art and constructs can be purified a commercially available kit. Either DNA or mRNA constructs can be generated.

A model payload oligonucleotide that includes a tetracycline repressor recognition sequence (tetO) and a Gaussia luciferase reporter (pCR-GLuc-11×TetO PCR product) as described in Example 1 can be used as a reporter construct.

Experimental methods for characterization with model payloads encoding a luciferase reporter can be carried out largely as described in the Examples above.

### Example 6—NLS Domain Optimization

This Example describes optimization and characterization of NLS domains. Specifically, this experiment describes optimization of NLS domains for use in TetR-based constructs, such as described in Example 1, as well as for use with a non-specific DNA binding domains described in Example 5. Exemplary NLSs for characterization can include:

SV40:
(SEQ ID NO: 66)
PKKKRKV
or
(SEQ ID NO: 33)
PSSDDEATADSQHSTPPKKKRKVEDPK
c-Myc:
(SEQ ID NO: 67)
PAAKRVKLD
Tus:
(SEQ ID NO: 68)
KLKIKRPVK

Concatemers or combinations of individual NLSs (for example, 2× c-Myc=PAAKRVKLD PAAKRVKLD; SEQ ID NO: 34), or SV40/c-Myc=PKKKRKVPAAKRVKLD; SEQ ID NO: 35) can also be generated and tested for efficacy.

Constructs including optimized NLS or a combination of NLSs can be generated using standard recombinant methods. Efficacy of these NLS sequences can be assess using reporter assays. For example, characterization with model payload oligonucleotides encoding a luciferase reporter can be carried out largely as described in the Examples above.

### Example 7—Transfection Efficiency with Co-Delivery of a DNA Oligonucleotide Comprising a Model Payload and an RNA Oligonucleotide Encoding a DNA Mimic Protein

The present Example demonstrates that co-delivery of a DNA oligonucleotide encoding a model payload to target cells with an RNA oligonucleotide comprising a sequence that encodes a DNA mimic protein can increase the expression and/or activity of the model payload in the target cells. Specifically, the present Example demonstrates that C2C12 myoblasts transfected with an mRNA oligonucleotide (e.g., a chemically-modified mRNA oligonucleotide) encoding a DNA mimic protein and a DNA plasmid encoding a reporter gene show a higher reporter expression level, as compared to that in cells transfected without an mRNA oligonucleotide encoding a DNA mimic protein.

Design and Synthesis of mRNA Oligonucleotides Comprising a Sequence that Encodes a DNA Mimic Polypeptide

PSI-BLAST (Altschul et al. (1997) Nucleic Acids Research, 25(17), pp. 3389-3402, which is incorporated by reference in its entirety) was used to search the NCBI GenBank database for proteins homologous to Ocr from T7 and ArdA from prokaryotic mobile elements. The following protein were synthesized as IDT gBlocks (first bullet=amino acid sequence of the protein, second bullet=sequence of gBlock ordered):

I. Ocr (from Bacteriophage T7)

An exemplary amino acid sequence:

(SEQ ID NO: 36)
MAMSNMTYNNVFDHAYEMLKENIRYDDIRDTDDLHDAIHMAADNAVPH
YYADIFSVMASEGIDLEFEDSGLMPDTKDVIRILQARIYEQLTIDLWE
DAEDLLNEYLEEVEEYEEDEE

An exemplary nucleotide sequence of gBlock:

(SEQ ID NO: 37)
caccATGgcaatgagcaatatgacatataacaatgttttcgaccacgc
ttatgagatgctcaaggaaaacatcagatatgacgacatacgcgacac
agatgacctgcacgacgcaattcatatggcagccgataatgccgtccc
tcattactatgcagacattttctcagttatggcatccgagggtattga
tctggagtttgaggactcaggccttatgccagacactaaggatgtcat
acggatcttgcaagcccggatctacgagcagcttactatagacctctg
ggaggacgcagaggacctcctgaacgagtatctggaggaagtcgaaga
gtacgaagaagacgaggaaTAGTGATAA

II. Exemplary Ocr Homolog: An KKK84065 Hypothetical Protein LCGC14_2787120 from Marine Sediment Metagenome

An exemplary amino acid sequence:

(SEQ ID NO: 38)
MSQSLYEIIKLAREELRGRAKDNKDETEPHDSIHEIADSSVPVYTGDL
LQLAADNLELATAKPELGPAFDGSPTPVNIVAANVFEAIEAGLWEEWK
EIESEREDAELEETG

An exemplary nucleotide sequence of gBlock:

(SEQ ID NO: 39)
caccATGagtcaaagtttgtacgagattattaaacttgctagggaaga
gcttcggggacgagcaaaggataacaaagatgagaccgagccccacga
ctctatccacgagattgctgattcatctgtgccagtctatacaggcga
tctcttgcagttggcagcagacaacttggagctggccacagctaaacc
agagcttggacccgcctttgatggcagtcccacaccagtcaacatcgt
ggctgctaatgtatttgaagccattgaagctgggctgtgggaagaatg
gaaggaaatcgagtcagaacgcgaagatgcagagttggaagaaactgg
cTAGTGATAA

III. Exemplary Ocr Homolog: KKN72305 Hypothetical Protein LCGC14_0412560 from Marine Sediment Metagenome

An exemplary amino acid sequence:

(SEQ ID NO: 40)
MKSDLQEILNDALDELKERMKDYPDEDADDVVSEIADSSVPVYYSDLLK
LASGCNDLATAEPECGPAFDGKPTPVNIIAANVYEAVDQHLRNYLSAI

An exemplary nucleotide sequence of gBlock:

(SEQ ID NO: 41)
caccATGaagtcagacttgcaagagattctcaatgatgcattggacga
attgaaggagcgaatgaaggattatcccgatgaggacgctgatgacgt
tgtaagcgagatagcagacagtagcgttccagtctactactctgacct
cttgaagctggcttccggctgcaatgaccttgctaccgctgaaccaga
atgcggaccagcattcgacgggaagccaacccctgtgaatattattgc
agctaatgtttacgaagcagtcgatcaacatctccgaaattacttgtc
agccattTAGTGATAA

IV. Antirestriction Protein ArdA from a Prokaryotic Mobile Element

An exemplary amino acid sequence:

(SEQ ID NO: 42)
MDDMQVYIANLGKYNEGELVGAWFTFPIDFEEVKEKIGLNDEYEEYAI
HDYELPFTVDEYTSIGELNRLWEMVSELPEELQSELSALLTHFSSIEE
LSEHQEDIIIHSDCDDMYDVARYYIEETGALGEVPASLQNYIDYQAYG
RDLDLSGTFISTNHGIFEIVY

An exemplary nucleotide sequence of gBlock:

(SEQ ID NO: 43)
caccATGgatgacatgcaagtttacattgcaaacttggggaagtacaa
tgagggggaactggtgggtgcatggttcaccttcccaatcgacttcga
ggaagtaaaagagaaaatcggacttaatgacgagtacgaagagtatgc
aatccacgattacgagctgccatttaccgtcgacgaatacactagcat
cggagaacttaataggctttgggaaatggtttccgagttgcccgaaga
actccagtcagaactttccgcacttcttacccacttcagcagtataga
agaactgtcagaacaccaagaagacatcataatacatagtgattgcga
tgatatgtacgatgtggccaggtactacatcgaagagactggggcttt
gggtgaggtccccgctagtctccaaaattatatagattaccaagccta
cggccgcgatcttgacctgtcagggacttttatttctactaaccacgg
aatcttcgagatcgtttacTAGTGATAA

Exemplary ArdA Homolog: EKC78327 Conjugative Transposon Protein from Human Gut Metagenome

An exemplary amino acid sequence:

(SEQ ID NO: 44)
MIDDMAVYIANLGKYNEGYLVGAWFTFPIDEEDVKEKIGLNEQYEEYAIH
DTDNFPIAIGEYVSIEELNEMYEMIEELPDYIVECLDEFISHYGTLEEVV
EHKDDIYYYPDCETMTDVACYYIDELQALGDIPPSLQNYIDYEAYGRDLD
MGGCFIETSRGMCEIPY

An exemplary nucleotide sequence of gBlock:

(SEQ ID NO: 45)
caccATGatagatgacatggcagtatacattgcaaatttgggtaagtata
acgaaggctatttggtgggtgcctggtttaccttccccattgacgaagaa
gatgttaaagaaaagataggactcaacgaacagtacgaagagtatgcaat
ccatgatactgataacttccccattgcaataggtgagtatgttagcatag
aggaactcaacgaaatgtacgaaatgattgaggaactgcccgactatatt
gtcgaatgtctcgatgagtttatttcacactacgggaccttggaggaagt
cgtcgaacacaaagacgatatttactactatccagattgtgagacaatga
ctgacgtagcctgttactacatagatgagttgcaagcattgggcgacata
ccacctagtctccaaaactacatcgactatgaagcatatggaagagattt
ggacatgggcgggtgtttcatcgagacaagccgagggatgtgcgaaattc
catatTAGTGATAA

V. Other ArdA (EKC62359.1 Antirestriction Protein ArdA from Human Gut Metagenome)

An exemplary amino acid sequence:

(SEQ ID NO: 46)
MVSQLYGIYRPQRPDILLSGADGESLARYLVQEVQLFGEVHPDLLNHIDY
AAIGRELETSENYLFTDNGIFYYR

An exemplary nucleotide sequence of gBlock:

(SEQ ID NO: 47)
caccATGgtatcacaactctacggaatctataggccccagcgcccagaca
cactcctttctggcgcagacggtgaaagtctcgcacggtacctcgtccag
gaggtgcagcttttcggagaagtgcatcccgacctgctgaaccacatcga
ctacgctgcaatcgggagggagctggagacttcagaaaattatctcttca
ctgataatggcattttctattaccggTAGTGATAA

VI. Other ArdA (EKC78842.1 Antirestriction Protein ArdA (Partial) from Human Gut Metagenome)

An exemplary amino acid sequence:

(SEQ ID NO: 48)
MSQDEYERFQAAMEIGDHIGSIQELINLTENLDCYDVYPDIHDHDDLGRY
YIEELDAMQVPEHLRNYIDYEAYGRDIALEESGQFIDLGYVRDTGDSFHE
YYDGERGSIPEEYRVMTFQDDIPEEEISEWAMDLAYDMDEFFRQNDPQYA
AEHPEEHAAKEEIYENLMAGRISALDEKLAALG

An exemplary nucleotide sequence of gBlock:

(SEQ ID NO: 49)
caccATGagtcaggacgaatacgagcgattccaggccgccatggaaatcg
gtgatcacacagggagcatacaagagttgatcaatcttaccgaaaatttg
gattgttacgacgtgtatcctgacatccatgaccatgatgatcttggaag
gtattatatagaagagctggatgcaatgcaagttcccgaacatctgagga
attacatagactatgaagcatatggccgggacatagccttggaagagtct
gggcagttcactgatttgggttatgtgagggacacaggcgattcctttca
cgagtactatgatggagaacgcggtagtattccagaggaatacagagtga
tgactttccaagatgatattcctgaagaagagatatccgaatgggcaatg
gatctcgcttatgacatggatgaatttttcagacaaaacgaccctcaata
cgccgcagaacacccagaggaacatgccgctaaggaagaaatatatgaaa
acctgatggcagggcggattagtgctttggatgagaagttggccgctctt
gggTAGTGATAA

Each synthesized construct was cloned into a DNA plasmid (e.g., pcDNA3.1), for example, using the pcDNA3.1 Directional TOPO Expression kit (Thermo Fisher). Plasmids was isolated, for example, using the ZymoPURE Plasmid Miniprep kit (Zymo Research). T7 templates were prepared by amplifying from the plasmids, for example, using pcDNA3.1_mRNA_fwd (CGAAATTAATACGACTCACTATAGGG; SEQ ID NO: 50) and pcDNA3.1_mRNA_rev (tttttttttttttttttttttttttttttttttttttttttttttttttttttttttttttt ttttttttttttttttttttttttttttttttttttttttttttttttttttttttttTCGA GGCTGATCAGCG; SEQ ID NO: 51), using a DNA polymerase (e.g., Herculase II polymerase (Agilent)). In one embodiment, the following cycling parameters were used:

95° C.-2 min

25 cycles:
95° C.-15 sec
60° C.-20 sec
72° C.-30 sec

72° C.-3 min

10° C.

Negative control template was generated by cloning the following gBlock into a DNA plasmid (e.g., pcDNA3.1) and amplifying with, e.g., primers T7_beta-glob fwd (aatggtTAATACGACTCACTATAGGGcaccttgttctttttgcagaag;SEQ ID NO: 52) and alpha-glob_pA_rev (TTTTTTTTTTTTTTTTTTTTTTTTTTTTTTTTTTTTTTTTTTTTTTTTTTTTTTTTTTTTTT TTTTTTTTTTTTTTTTTTTTTTTTTTTTTTTTTTTTTTTTTTTTTTTTTTTTTTTTTTcttc ctactcaggctttattc; SEQ ID NO: 53).

An exemplary nucleotide sequence of gBlock for a negative control template:

(SEQ ID NO: 54)
CACcttgttctttttgcagaagctcagaataaacgctcaactttggccTA
GCCGCTATAATTGTTTCTATGCCGAGTAATGAGAACAACCACACCATAGC
GATTTAACGCAGCGCCTCGGAATACCGTTTTAGCAGGCGCTTGCTAAGAC
CATTGCGAATTCCAGGTATCGTGTATGTAGCGTAGGGCCATACGCAAGTT
AAACTGCTAGGAAACCGCGTTTCTACGACCGGTGCATAATTTAATTTCGC
TGACGTGATGACATTCCTGCTAATGCCTCACCTGTCGGATCCCTCTCGTG
ATAGGGTAGTTGGACATGTCCTTGTAAGATATAACAAGAGCCTGCCTGTT
TAATGATCTCACGGCGAAAGTCGGGGAGACAGCAGCGGCTGCAGACATTA
TATCGCAATAATACTAAGGTGAGATAACTCCGTAATTGACTACGCATTTC
TCTAGACTTTACTTGACCAGATACAGTGACTTTGACACGTTTATGGATTA
CAGCAATCACATCCAAGACTGCCTATGGAGGAAGCAACTCTTGAGTGTTA
ATATGTTGACTCCTGTATTAGGGATGCAGGTAGTAGATGAGTGCAGGGAC
ACCGAGGTTAAGTACATTACCCTCTCATAGGAGGTGTTCTAGATCACCAT
ACCACCATATTATTCGAGCATGACATTATCTGCGCTGTCCCCATCCTAGT
AGTCATTATTCCTATTACGCTTTTGAGTGACTGGTGACGGAgctgccttc
tgcggggcttgccttctggccatgcccttcttctctcccttgcacctgta
cctcttggtctttgaataaagcctgagtaggaag

The PCR products were cleaned up, e.g., using DNA Clean & Concentrator-5 (Zymo Research), digested with Dpn I (New England Biolabs), and exposed to 1× RNASecure (Thermo Fisher) at 60° C. mRNA was synthesized using the MEGAscript T7 Transcription Kit (Thermo Fisher). The synthesis reactions consisted of 25 μmol/μL template DNA, 6 mM RNA cap structure analog (e.g., 3‘-O-Me-m’G(5′)ppp(5′)G; Anti-Reverse Cap Analogue (ARCA)) (TriLink Biotechnologies), 7.5 mM Ni-methylpseudouridine-5′-triphosphate (TriLink Biotechnologies), 1.5 mM GTP, 7.5 mM ATP, 7.5 mM CTP, 1× reaction buffer, and 0.1 μL/μL Enzyme Mix. The mRNA transcription reactions were carried out for 2 hrs at 37° C. mRNA products were subsequently treated with TURBO DNase (Thermo Fisher) and purified using the MEGAclear Transcription Clean-Up Kit (Thermo Fisher), eluting into 0.1 mM EDTA pH 8.

The yield was quantified using a NanoDrop spectrophotometer (Thermo Fisher). The purified products were then treated with 1×RNAsecure at 60° C., and the integrity of the synthesized products was confirmed using 6.66% formaldehyde/1% MOPS/1% agarose gels stained with SYBR Gold (Thermo Fisher).

### Cell Transfection

C2C12 cells (ATCC) were cultured in high glucose Dulbecco's Modified Eagle Medium (Thermo Fisher) supplemented with 15% dialyzed fetal bovine serum and maintained at 37° C. and 5% CO2. Cells were plated in a 24-well plate at 20,000 cells one day prior to transfection. One day after plating cells were transfected with 500 ng of mRNA oligonucleotides encoding either one of the DNA mimic proteins (e.g., as described above) or a random sequence (e.g., a negative control sequence). Transfections were carried out, e.g., using Lipofectamine MessengerMAX (Thermo Fisher) at 1.5 μL reagent per g mRNA. One day after the mRNA transfection the cells were split 1:5 into a fresh 24-well plate, e.g., using TrypLE Express (Thermo Fisher). One day after splitting the cells were transfected with 200 ng pCMV-GLuc 2 (New England Biolabs), e.g., using Lipofectamine 3000 (Thermo Fisher) at 1.5 μL reagent per g DNA. One day after the DNA transfection the cells were assayed for luciferase activity, e.g., using the Pierce Gaussia Luciferase Glow Assay kit (Thermo Fisher).

### Results

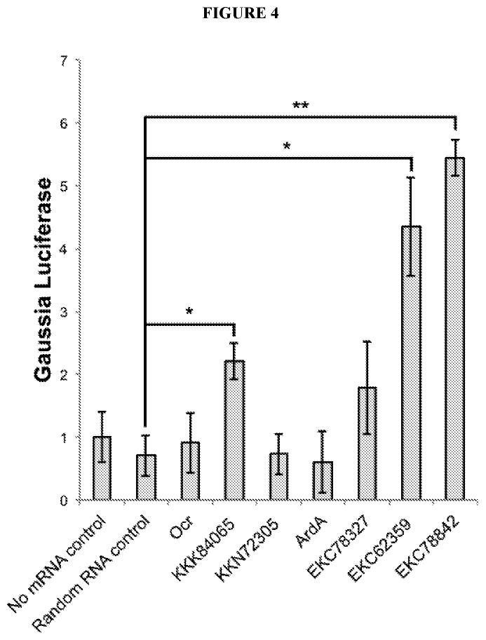

FIG. 4 shows the Gaussia expression signal of each sample subtracted from the background reading from untreated cells (i.e., cells that were not treated with mRNA oligonucleotides encoding DNA mimic polypeptides or DNA oligonucleotides encoding a model payload) and normalized by the signal when mRNA control oligonucleotides were absent. FIG. 4 shows that co-delivery of a DNA oligonucleotide encoding a model payload with an RNA oligonucleotide (e.g., a mRNA oligonucleotide) comprising a sequence that encodes a DNA mimic polypeptide) can increase the expression level of the model payload in target cells.

### Example 8—Transfection Efficiency of Co-Delivery of an RNA Oligonucleotide Comprising a Sequence that Encodes a Model Payload with an RNA Oligonucleotide Comprising a Sequence that Encodes a US11 Polypeptide

The present Example describes synthesis of an RNA oligonucleotide comprising a sequence that encodes an exemplary US11 polypeptide and an RNA oligonucleotide comprising a model payload sequence and further demonstrates that co-delivery of an RNA oligonucleotide comprising a model payload sequence with an RNA oligonucleotide comprising a sequence that encodes an exemplary US11 polypeptide can increase expression of the model payload. While this study assessed expression of a payload in target cells when an RNA oligonucleotide comprising a sequence that encodes a model payload (e.g., a model reporter polypeptide) was delivered to target cells following delivery of an RNA oligonucleotide comprising a sequence that encodes a US11 polypeptide, similar technical effects can also be observed when both an RNA polynucleotide comprising a model payload sequence and an RNA polynucleotide encoding a US11 polypeptide are delivered concurrently to the target cells (see Example 9).

Preparation of an RNA Oligonucleotide Comprising a Sequence that Encodes an Exemplary Model Payload Sequence

Firefly Luciferase mRNA Synthesis:

The luc2 gene encoding an optimized version of firefly luciferase was amplified from pGL4.10[luc2] (Promega) with Luc2_fwd (shown below) and Luc2_rev (shown below) using Herculase II polymerase (Agilent) with an annealing temperature of 70° C. and with 250 mM betaine supplementation. The PCR product was cleaned up using DNA Clean & Concentrator-5 (Zymo Research) and digested with Dpn I (New England Biolabs). The digested PCR product as then amplified with T7-AGG_fwd and 120 pA_rev using Herculase II polymerase with an annealing temperature of 50° C. and with 1 M betaine supplementation. The secondary PCR product was cleaned up using DNA Clean & Concentrator-5 and used as a template for T7 transcription. Specifically, the HighScribe T7 High Yield (New England Biolabs) was used to set up reactions consisting of 40 ng/μL luc2 template, 10 mM of each nucleotide (ATP, GTP, UTP, CTP), 10 mM of CleanCap Reagent AG (Trilink Biotech), 1× T7 buffer, and 0.1 μL/μL T7 RNA polymerase mix. The mRNA transcription reactions were carried out for 2 hrs at 37° C. mRNA products were subsequently treated with TURBO DNase (Thermo Fisher) and purified using the MEGAclear Transcription Clean-Up Kit (Thermo Fisher), eluting into 0.1 mM EDTA pH 8. The yield was quantified using a NanoDrop spectrophotometer (Thermo Fisher). The purified products were then treated with 1×RNAsecure (Thermo Fisher) and heated to 60° C. in order to inactivate any contaminating RNAses. The integrity of the synthesized products were confirmed using 2% EX gels (Thermo Fisher).

The sequences of exemplary primers used are shown as follows:

Luc2_fwd:
(SEQ ID NO: 55)
CTTGTTCTTTTTGCAGAAGCTCAGAATAAACGCTCAACTTTGGCCACCat
ggaagatgccaaaaacattaagaagggc
Luc2_rev:
(SEQ ID NO: 56)
AGAATGTGAAGAAACTTTCTTTTTATTAGGAGCAGATACGAATGGCTACA
TTTTGGGGGACAACATTTTGTAAAGTGTAAGTTGGTATTATGTAGCTTAG
AGACTCCATTCGGGTGTTCTTGAGGCTGGTCTATCATTAcacggcgatct
tgccgcc
T7-AGG_fwd:
(SEQ ID NO: 57)
gaattTAATACGACTCACTATAAGGcttgttctttttgcagaagc
120pA_rev:
(SEQ ID NO: 58)
TTTTTTTTTTTTTTTTTTTTTTTTTTTTTTTTTTTTTTTTTTTTTTTTTT
TTTTTTTTTTTTTTTTTTTTTTTTTTTTTTTTTTTTTTTTTTTTTTTTTT
TTTTTTTTTTTTTTTTTTTTagaatgtgaagaaactttctttttattag

An exemplary sequence of a luc2 mRNA product is shown as follows:

(SEQ ID NO: 59)
AGGCUUGUUCUUUUUGCAGAAGCCUUGUUCUUUUUGCAGAAGCUCAGAAU
AAACGCUCAACUUUGGCCACCAUGGAAGAUGCCAAAAACAUUAAGAAGGG
CCCAGCGCCAUUCUACCCACUCGAAGACGGGACCGCCGGCGAGCAGCUGC
ACAAAGCCAUGAAGCGCUACGCCCUGGUGCCCGGCACCAUCGCCUUUACC
GACGCACAUAUCGAGGUGGACAUUACCUACGCCGAGUACUUCGAGAUGAG
CGUUCGGCUGGCAGAAGCUAUGAAGCGCUAUGGGCUGAAUACAAACCAUC
GGAUCGUGGUGUGCAGCGAGAAUAGCUUGCAGUUCUUCAUGCCCGUGUUG
GGUGCCCUGUUCAUCGGUGUGGCUGUGGCCCCAGCUAACGACAUCUACAA
CGAGCGCGAGCUGCUGAACAGCAUGGGCAUCAGCCAGCCCACCGUCGUAU
UCGUGAGCAAGAAAGGGCUGCAAAAGAUCCUCAACGUGCAAAAGAAGCUA
CCGAUCAUACAAAAGAUCAUCAUCAUGGAUAGCAAGACCGACUACCAGGG
CUUCCAAAGCAUGUACACCUUCGUGACUUCCCAUUUGCCACCCGGCUUCA
ACGAGUACGACUUCGUGCCCGAGAGCUUCGACCGGGACAAAACCAUCGCC
CUGAUCAUGAACAGUAGUGGCAGUACCGGAUUGCCCAAGGGCGUAGCCCU
ACCGCACCGCACCGCUUGUGUCCGAUUCAGUCAUGCCCGCGACCCCAUCU
UCGGCAACCAGAUCAUCCCCGACACCGCUAUCCUCAGCGUGGUGCCAUUU
CACCACGGCUUCGGCAUGUUCACCACGCUGGGCUACUUGAUCUGCGGCUU
UCGGGUCGUGCUCAUGUACCGCUUCGAGGAGGAGCUAUUCUUGCGCAGCU
UGCAAGACUAUAAGAUUCAAUCUGCCCUGCUGGUGCCCACACUAUUUAGC
UUCUUCGCUAAGAGCACUCUCAUCGACAAGUACGACCUAAGCAACUUGCA
CGAGAUCGCCAGCGGCGGGGCGCCGCUCAGCAAGGAGGUAGGUGAGGCCG
UGGCCAAACGCUUCCACCUACCAGGCAUCCGCCAGGGCUACGGCCUGACA
GAAACAACCAGCGCCAUUCUGAUCACCCCCGAAGGGGACGACAAGCCUGG
CGCAGUAGGCAAGGUGGUGCCCUUCUUCGAGGCUAAGGUGGUGGACUUGG
ACACCGGUAAGACACUGGGUGUGAACCAGCGCGGCGAGCUGUGCGUCCGU
GGCCCCAUGAUCAUGAGCGGCUACGUUAACAACCCCGAGGCUACAAACGC
UCUCAUCGACAAGGACGGCUGGCUGCACAGCGGCGACAUCGCCUACUGGG
ACGAGGACGAGCACUUCUUCAUCGUGGACCGGCUGAAGAGCCUGAUCAAA
UACAAGGGCUACCAGGUAGCCCCAGCCGAACUGGAGAGCAUCCUGCUGCA
ACACCCCAACAUCUUCGACGCCGGGGUCGCCGGCCUGCCCGACGACGAUG
CCGGCGAGCUGCCCGCCGCAGUCGUCGUGCUGGAACACGGUAAAACCAUG
ACCGAGAAGGAGAUCGUGGACUAUGUGGCCAGCCAGGUUACAACCGCCAA
GAAGCUGCGCGGUGGUGUUGUGUUCGUGGACGAGGUGCCUAAAGGACUGA
CCGGCAAGUUGGACGCCCGCAAGAUCCGCGAGAUUCUCAUUAAGGCCAAG
AAGGGCGGCAAGAUCGCCGUGUAAUGAUAGACCAGCCUCAAGAACACCCG
AAUGGAGUCUCUAAGCUACAUAAUACCAACUUACACUUUACAAAAUGUUG
UCCCCCAAAAUGUAGCCAUUCGUAUCUGCUCCUAAUAAAAAGAAAGUUUC
UUCACAUUCUAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAA

Preparation of an RNA Oligonucleotide Comprising a Sequence that Encodes an Exemplary US11 Polypeptide

US11 mRNA Synthesis:

The US11 gene containing 5′ and 3′ UTRs was synthesized, for example, as a gBlock (Integrated DNA Technologies) and amplified, e.g., with T7-AGG_fwd and 120 pA_rev using a polymerase (e.g., Herculase II polymerase) with an annealing temperature of 50° C. and with 1 M betaine supplementation. The PCR product was cleaned up, e.g., using DNA Clean & Concentrator-5, and used as a template for T7 transcription. The transcription reactions were carried out, e.g., using HighScribe T7 High Yield as described above for luc2 mRNA synthesis, except using 20 ng/μL US11 template. mRNA products were DNAse digested as described for luc2 mRNA synthesis. Purification was carried out, e.g., using Dynabeads Oligo (dT)25 (SEQ ID NO: 69) (Thermo Fisher). For example, the 0.2375 μL of the magnetic beads were added per μL of T7 synthesis reaction in Binding Buffer (1×RNAsecure, 1M LiCl, 2 mM EDTA, 20 mM Tris-Cl pH 7.5). The mixture was heated to 60° C. to denature the mRNA, cooled on ice, and incubated at room temperature for 5 min. The mRNA-bound beads were then washed using Wash Buffer (1× RNAsecure, 150 mM LiCl, 1 mM EDTA, 10 mM Tris-Cl pH 7.5). Bead-bound mRNA was eluted into Elution Buffer (1×RNAsecure, 1 mM EDTA, 10 mM Tris-Cl pH 7.5) by heating to 80° C. for 2 min. The yield was quantified using the QuantiFlour RNA System (Promega). The integrity of the synthesized products were confirmed using 1% EX gels.

An exemplary sequence of the US11 gBlock is shown as follows:

(SEQ ID NO: 60)
CTTGTTCTTTTTGCAGAAGCTCAGAATAAACGCTCAACTTTGGCCACCat
gagccagacccaacccccggccccagttgggccgggcgacccagatgttt
acttaaaaggcgtgccgtccgccggcatgcaccccagaggtgttcacgca
cctcgaggacacccgcgcatgatctccggacccccgcaacggggtgataa
cgatcaagcggcggggcaatgtggagattcgggtctactacgagtcggtg
cggacactacgatctcgaagccatctgaagccgtccgaccgccaacaatc
cccaggacaccgcgtgttccccgggagccccgggttccgcgaccaccccg
agaacctagggaacccagagtaccgcgagctcccagagaccccagggtac
cgcgtgaccccagggatccacgacaaccGcgTtcCccAagggagccccgg
tctcccCgTgaAccccggtctcccagggagccccggaccccacgcacccc
ccgcgaaccacgtacggctcgcggTtctgtaTAATGATAGACCAGCCTCA
AGAACACCCGAATGGAGTCTCTAAGCTACATAATACCAACTTACACTTTA
CAAAATGTTGTCCCCCAAAATGTAGCCATTCGTATCTGCTCCTAATAAAA
AGAAAGTTTCTTCACATTCT

An exemplary sequence of a US11 mRNA product is shown as follows:

(SEQ ID NO: 61)
AGGCUUGUUCUUUUUGCAGAAGCCUUGUUCUUUUUGCAGAAGCUCAGAAU
AAACGCUCAACUUUGGCCACCAUGAGCCAGACCCAACCCCCGGCCCCAGU
UGGGCCGGGCGACCCAGAUGUUUACUUAAAAGGCGUGCCGUCCGCCGGCA
UGCACCCCAGAGGUGUUCACGCACCUCGAGGACACCCGCGCAUGAUCUCC
GGACCCCCGCAACGGGGUGAUAACGAUCAAGCGGCGGGGCAAUGUGGAGA
UUCGGGUCUACUACGAGUCGGUGCGGACACUACGAUCUCGAAGCCAUCUG
AAGCCGUCCGACCGCCAACAAUCCCCAGGACACCGCGUGUUCCCCGGGAG
CCCCGGGUUCCGCGACCACCCCGAGAACCUAGGGAACCCAGAGUACCGCG
AGCUCCCAGAGACCCCAGGGUACCGCGUGACCCCAGGGAUCCACGACAAC
CGCGUUCCCCAAGGGAGCCCCGGUCUCCCCGUGAACCCCGGUCUCCCAGG
GAGCCCCGGACCCCACGCACCCCCCGCGAACCACGUACGGCUCGCGGUUC
UGUAUAAUGAUAGACCAGCCUCAAGAACACCCGAAUGGAGUCUCUAAGCU
ACAUAAUACCAACUUACACUUUACAAAAUGUUGUCCCCCAAAAUGUAGCC
AUUCGUAUCUGCUCCUAAUAAAAAGAAAGUUUCUUCACAUUCUAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAA

Exemplary Treatment of Target Cells with an RNA Oligonucleotide Comprising a Model Payload Sequence and an RNA Oligonucleotide Comprising a Sequence that Encodes a US11 Polypeptide

A549 MAVS-transfection: Mitochondrial antiviral signaling (MAVS) knock-out cancer cells such as lung cancer cells (e.g., A549-Dual KO-MAVS cells (InvivoGen)) were cultured, e.g., in high glucose GlutaMAX Dulbecco's Modified Eagle Medium (Thermo Fisher) supplemented with 10% heat-inactivated fetal bovine serum, 100 units/mL penicillin, 100 μg/mL streptomycin, 10 μg/mL blasticidin, and 100 μg/mL zeocin and maintained at 37° C. and 5% CO₂. Cells were plated in a 96-well plate at 4,000 cells/well in antibiotic-free culture media one day prior to treatment (e.g., transfection). Variable amounts of a mRNA oligonucleotide comprising a sequence that encodes a US11 polypeptide was delivered to cells per well, e.g., via transfections, e.g., using Lipofectamine MessengerMAX at a 1.5 uL reagent per g mRNA. An appropriate amount of a mRNA oligonucleotide comprising a sequence that encodes a model payload, e.g., a reporter polypeptide (e.g., 300 ng of a mRNA oligonucleotide comprising a sequence that encodes luc2) was then delivered to the cells, e.g., by transfections, following delivery of the mRNA oligonucleotide comprising a sequence that encodes a US11 polypeptide. For example, a mRNA oligonucleotide comprising a sequence that encodes a model payload, e.g., luc2, was delivered to the cells, e.g., 1 day following delivery of the mRNA oligonucleotide comprising a sequence that encodes a US11 polypeptide. Levels of a model payload (e.g., luciferase levels) were assayed, e.g., 1 day following a payload (e.g., luc2) transfection, for example, using the ONE-Glo+Tox Luciferase Reporter and Cell Viability assay (Promega).

### Results

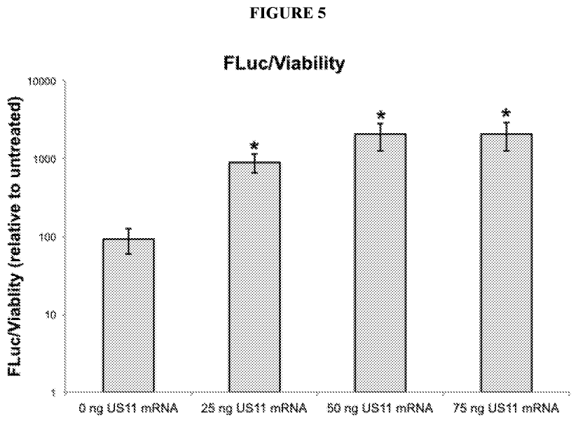

As shown in FIG. 5, expression of a model payload (e.g., luciferase) in the target cells was increased by co-delivery of a mRNA oligonucleotide comprising a model payload sequence (e.g., luc2) with a mRNA oligonucleotide comprising a sequence that encodes a US11 polypeptide. For example, a significant (p<0.05, n=2-3 replicate mRNA preparations and transfections) improvement in firefly luciferase expression was observed in the cells with co-expression of US11.

### Example 9—Effects of Co-Delivery of a RNA Oligonucleotide Comprising a Payload Sequence with an RNA Oligonucleotide Comprising a Sequence that Encodes a US11 Polypeptide on Target Cells

The present Example demonstrates that co-delivery of an RNA oligonucleotide comprising a payload sequence to target cells with an RNA oligonucleotide comprising a sequence that encodes an exemplary US11 polypeptide can reduce non-specific toxicity induced in the target cells by the RNA oligonucleotide comprising a model payload sequence. The present Example further demonstrates that co-delivery of an RNA oligonucleotide comprising a model payload sequence to target cells with an RNA oligonucleotide comprising a sequence that encodes an exemplary US11 polypeptide can improve viability of the target cells upon delivery of the RNA oligonucleotide comprising the model payload sequence into the target cells. While this study assessed non-specific toxicity and cell viability when both an RNA oligonucleotide comprising a sequence that encodes a model payload (e.g., a scramble sequence as a negative control in this Example) and an RNA oligonucleotide comprising a sequence that encodes a US11 polypeptide was concurrently delivered to target cells, similar technical effects can be exerted on the target cells when an RNA oligonucleotide comprising a sequence that encodes a model payload is delivered to target cells following delivery of an RNA oligonucleotide comprising a sequence that encodes a US11 polypeptide. See Example 8. Further, this Example demonstrates that similar technical effects were exerted on cells that have been previously treated (e.g., transfected) at least one or more (e.g., once, twice, or three times) with one or more oligonucleotides (e.g., RNA oligonucleotides encoding a payload).

Preparation of an RNA Oligonucleotide Comprising a Sequence that Encodes an Exemplary US11 Polypeptide

US11 mRNA synthesis: The US11 gBlock (e.g., as described in Example 8) was cloned into pCR II-Blunt-TOPO using the Zero Blunt TOPO PCR Cloning Kit (Thermo Fisher). T7 template was then generated by amplifying the cloned US11 gene with T7-AGG_fwd and 120 pA_rev using Herculase II polymerase with an annealing temperature of 50° C. The template was purified up using DNA Clean & Concentrator-25 (Zymo Research), digested with Dpn I, further purified with DNA Clean & Concentrator-5, and treated with 1× RNAsecure. The transcription reactions were carried out using HighScribe T7 High Yield, e.g, as described in Example 8. mRNA products were DNAse digested, purified, and characterized, e.g, as described for luc2 mRNA synthesis in Example 8.

### Preparation of an RNA Oligonucleotide Comprising a Payload Sequence (e.g., a Scramble Control Sequence)

An exemplary sequence of a synthesized scramble control 1 gBlock is shown as follows:

(SEQ ID NO: 62)
taaCTTGTTCTTTTTGCAGAAGCTCAGAATAAACGCTCAACTTTGGCCAC
Catgctagtcaacctccttaaggacgagcgtcgggcgaacatccgcgcca
aacggcacttgacactgcaggtccagatcaatcggcaacaagccgtcaag
gaaacctctgtagcattgaggacggagttgcacaacctgaggctaatcac
attgaataaagaaccgcaatttgttaaagccaaaaaccgatcctttatcg
acagggagcaggagtctaaattgtgtgaaaacgcaaagtaccagagcgag
cttcccaagattaaagaagaggaaTAATGATAGACCAGCCTCAAGAACAC
CCGAATGGAGTCTCTAAGCTACATAATACCAACTTACACTTTACAAAATG
TTGTCCCCCAAAATGTAGCCATTCGTATCTGCTCCTAATAAAAAGAAAGT
TTCTTCACATTCT

An exemplary sequence of a synthesized scramble control 2 gBlock is shown as follows:

(SEQ ID NO: 63)
taaCTTGTTCTTTTTGCAGAAGCTCAGAATAAACGCTCAACTTTGGCCAC
Catgaacaagaactctatccggcaatctcttaaaaacgttcggttggacg
aagtcgcaaacgcacacttgcaacagagggaagtcaaaccgatcgacatc
aagcgcctgagcaaagccgagaacaaatttcaatacttgagggtcgaaaa
gcagacactattggccgagcccaaagagtgtacggagcgtaataaggagt
ttcaggtagaactgaggacacgagagcagcgggcccacttgattacccta
cttctcgaaatcaaagcgaattccTAATGATAGACCAGCCTCAAGAACAC
CCGAATGGAGTCTCTAAGCTACATAATACCAACTTACACTTTACAAAATG
TTGTCCCCCAAAATGTAGCCATTCGTATCTGCTCCTAATAAAAAGAAAGT
TTCTTCACATTCT

An exemplary sequence of a mRNA product of scramble control 1 is shown as follows:

(SEQ ID NO: 64)
AGGCUUGUUCUUUUUGCAGAAGCCUUGUUCUUUUUGCAGAAGCUCAGAAU
AAACGCUCAACUUUGGCCACCAUGCUAGUCAACCUCCUUAAGGACGAGCG
UCGGGCGAACAUCCGCGCCAAACGGCACUUGACACUGCAGGUCCAGAUCA
AUCGGCAACAAGCCGUCAAGGAAACCUCUGUAGCAUUGAGGACGGAGUUG
CACAACCUGAGGCUAAUCACAUUGAAUAAAGAACCGCAAUUUGUUAAAGC
CAAAAACCGAUCCUUUAUCGACAGGGAGCAGGAGUCUAAAUUGUGUGAAA
ACGCAAAGUACCAGAGCGAGCUUCCCAAGAUUAAAGAAGAGGAAUAAUGA
UAGACCAGCCUCAAGAACACCCGAAUGGAGUCUCUAAGCUACAUAAUACC
AACUUACACUUUACAAAAUGUUGUCCCCCAAAAUGUAGCCAUUCGUAUCU
GCUCCUAAUAAAAAGAAAGUUUCUUCACAUUCUAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAA

An exemplary sequence of a mRNA product of scramble control 2 is shown as follows:

(SEQ ID NO: 65)
AGGCUUGUUCUUUUUGCAGAAGCCUUGUUCUUUUUGCAGAAGCUCAGAAU
AAACGCUCAACUUUGGCCACCAUGAACAAGAACUCUAUCCGGCAAUCUCU
UAAAAACGUUCGGUUGGACGAAGUCGCAAACGCACACUUGCAACAGAGGG
AAGUCAAACCGAUCGACAUCAAGCGCCUGAGCAAAGCCGAGAACAAAUUU
CAAUACUUGAGGGUCGAAAAGCAGACACUAUUGGCCGAGCCCAAAGAGUG
UACGGAGCGUAAUAAGGAGUUUCAGGUAGAACUGAGGACACGAGAGCAGC
GGGCCCACUUGAUUACCCUACUUCUCGAAAUCAAAGCGAAUUCCUAAUGA
UAGACCAGCCUCAAGAACACCCGAAUGGAGUCUCUAAGCUACAUAAUACC
AACUUACACUUUACAAAAUGUUGUCCCCCAAAAUGUAGCCAUUCGUAUCU
GCUCCUAAUAAAAAGAAAGUUUCUUCACAUUCUUUUUUUUUUUUUUUUUU
UUUUUUUUUUUUUUUUUUUUUUUUUUUUUUUUUUUUUUUUUUUUAAAAAA
AAAAAAAAAAAAAAAAAAAAAAAAAAAAAAAAAAAAAAAAAAAAAAAAAA
AAA

Exemplary Treatment of Target Cells with an RNA Oligonucleotide Comprising a Model Payload Sequence and an RNA Oligonucleotide Comprising a Sequence that Encodes a US11 Polypeptide

A549 MAVS-Transfection:

Mitochondrial antiviral signaling (MAVS) knock-out cancer cells such as lung cancer cells (e.g., A549-Dual KO-MAVS cells (InvivoGen)) were cultured, e.g., as described in Example 8. The cells were plated in a 24-well plate at 25,000 cells/well in culture media one day prior to transfection. The cells were treated (e.g., by transfection) with either (i) a mRNA oligonucleotide comprising a payload sequence (e.g., 250 ng mRNA oligonucleotide comprising a scramble control sequence), or (ii) a mixture of a mRNA oligonucleotide comprising a payload sequence (e.g., 175 ng mRNA oligonucleotide comprising a scramble control sequence) and a mRNA oligonucleotide comprising a sequence that encodes a US11 polypeptide (e.g., 75 ng mRNA oligonucleotide comprising a sequence that encodes a US11 polypeptide). An exemplary mixture comprised 250 ng total mRNA oligonucleotides with 25% US11 spike-in. The mRNA oligonucleotides were delivered to cells, e.g., by transfection. For example, transfections were carried out, e.g., using Lipofectamine MessengerMAX at a 1.5 μL reagent per g mRNA. On day 5 following delivery (e.g., transfection), cells were collected, e.g., using TrypLE Express (Thermo Fisher). Live cells were counted, e.g., using a Countess II Automated Cell Counter (Thermo Fisher) and EVE Cell Counting Slides (NanoEnTek), and a portion of the collected cells (e.g., 25% of the collected cells) were plated to a fresh 24-well plate. The plated cells were treated again (e.g., by transfection), e.g., on day 1 following plating, and were passaged and counted, e.g., on day 6 following plating. The process was repeated for multiple treatments (e.g., a total of at least 3 repeated transfections).

### Results

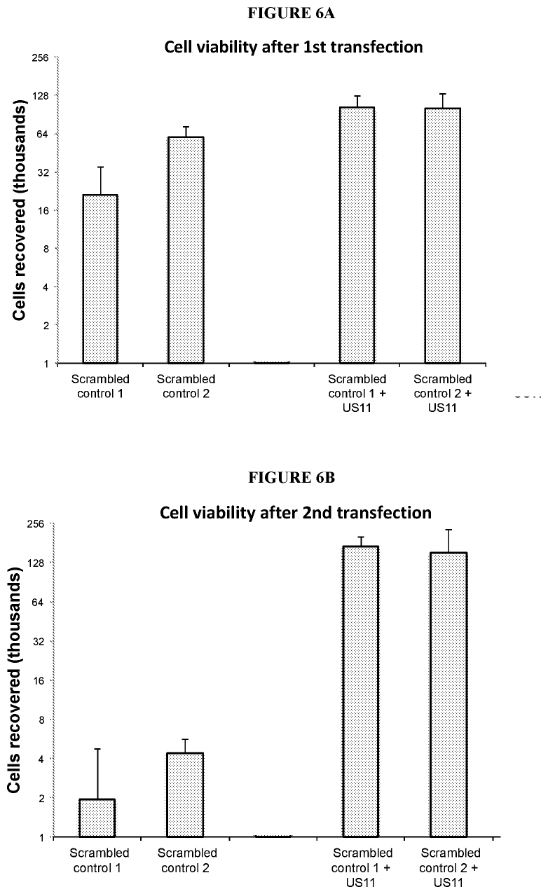

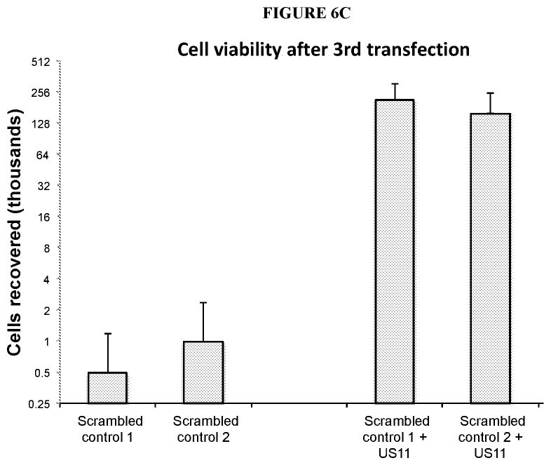

As shown in FIGS. 6A-6C, delivery of mRNA oligonucleotides encoding a negative control (e.g., scrambled sequences) alone (e.g., in the absence of mRNA oligonucleotides encoding a US11 polypeptide) induced cell death and thus lowered cell viability upon treatment (e.g., transfection). This data indicate that delivery of mRNA oligonucleotides at a tested dose into target cells can induce non-specific toxicity. However, an equivalent total amount of mRNA oligonucleotides containing a 25% mRNA oligonucleotides encoding a US11 polypeptide resulted in improved cell viability. Such technical effects were also observed in cells after repeated treatments (e.g., by transfections). It is noted that A549 cells used in this study have the G12S MYC mutation (Mahoney et al. (2009) British Journal of Cancer 100(2), p. 370, which is incorporated by reference in its entirety).

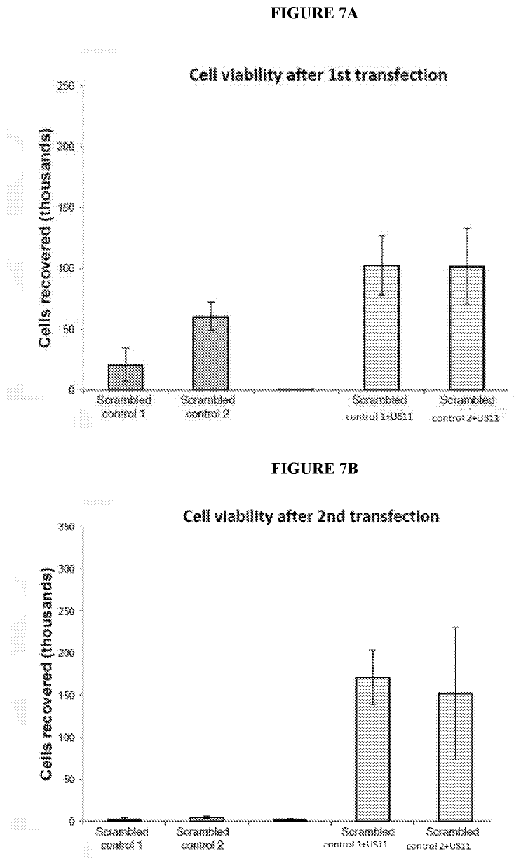

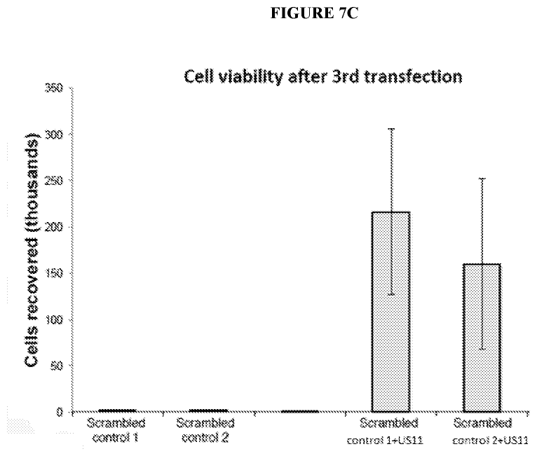

Similar studies as described in this Example were performed with an RNA oligonucleotide comprising a sequence that encodes a negative control (e.g., scramble sequence) and an RNA oligonucleotide comprising a sequence that encodes a US11 polypeptide, according to another embodiment described herein. Similar to FIGS. 6A-6C, cells treated by co-delivery of a mRNA oligonucleotide encoding a negative control and a mRNA oligonucleotide encoding a US11 polypeptide had significantly higher cell viability than those treated by delivery of a mRNA oligonucleotide encoding a negative control (e.g., a scramble sequence) alone (e.g., in the absence of a mRNA oligonucleotide encoding a US11 polypeptide). Such technical effects were also observed in cells after repeated treatments (e.g., by transfections). Results are shown in FIGS. 7A-7C.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1. A nucleic acid expression system comprising:
(i) an oligonucleotide comprising a payload sequence, and
(ii) at least one oligonucleotide comprising a sequence that encodes a helper polypeptide that confers one or more of the following characteristics:
(a) enhancing expression and/or activity of an oligonucleotide comprising a payload sequence in a target cell;
(b) enhancing nuclear import of an oligonucleotide comprising a payload sequence in a target cell;
(c) enhancing persistence or uptake of an oligonucleotide comprising a payload sequence in a target cell;
(d) enhancing viability of a target cell upon contacting with an oligonucleotide comprising a payload sequence; and
(e) reducing non-specific toxicity induced in a target cell by an oligonucleotide comprising a payload sequence.

2. The nucleic acid expression system of claim 1, wherein the oligonucleotide comprising a payload sequence and/or the at least one oligonucleotide comprising a sequence that encodes a helper polypeptide is a synthetic oligonucleotide.

3. The nucleic acid expression system of claim 2, wherein the oligonucleotide comprising a payload sequence and/or the at least one oligonucleotide comprising a sequence that encodes a helper polypeptide is a DNA oligonucleotide.

4. The nucleic acid expression system of claim 2, wherein the oligonucleotide comprising a payload sequence and/or the at least one oligonucleotide comprising a sequence that encodes a helper polypeptide is an RNA oligonucleotide (e.g., a messenger RNA (mRNA) oligonucleotide).

5. The nucleic acid expression system of claim 2, wherein one of the following conditions applies:
(a) the oligonucleotide comprising a payload sequence is a DNA oligonucleotide and the at least one oligonucleotide comprising a sequence that encodes a helper polypeptide is a DNA oligonucleotide;
(b) the oligonucleotide comprising a payload sequence is a DNA oligonucleotide and the at least one oligonucleotide comprising a sequence that encodes a helper polypeptide is an RNA oligonucleotide (e.g., a mRNA oligonucleotide); and
(c) the oligonucleotide comprising a payload sequence is an RNA oligonucleotide and the at least one oligonucleotide comprising a sequence that encodes a helper polypeptide is an RNA oligonucleotide (e.g., a mRNA oligonucleotide).

6. The nucleic acid expression system of claim 1, wherein the oligonucleotide comprising a payload sequence and/or the at least one oligonucleotide comprising a sequence that encodes a helper polypeptide are part of a vector.

7. A nucleic acid expression system that includes (i) an oligonucleotide comprising a payload sequence and (ii) a composition that delivers at least one helper polypeptide.

8. The nucleic acid expression system of claim 7, wherein the composition that delivers a helper polypeptide is or comprises (i) an oligonucleotide comprising a sequence that encodes a helper polypeptide and/or (ii) a helper polypeptide.

9. The nucleic acid expression system of claim 1, wherein the helper polypeptide is or comprises one or more of the following: a nuclear localization signal (NLS) polypeptide, a DNA mimic polypeptide, a modulator of innate immunity, and a synthetic cell surface receptor polypeptide.

10. The nucleic acid expression system of claim 1, wherein the helper polypeptide is or comprises a NLS polypeptide, optionally wherein the NLS polypeptide is (a) an SV40 NLS polypeptide or variant thereof; or (b) from EGL-13 polypeptide, c-Myc polypeptide, NLP polypeptide or TUS polypeptide.

11. The nucleic acid expression system of claim 1, wherein the helper polypeptide is or comprises a DNA mimic polypeptide, optionally wherein the DNA mimic polypeptide is selected from any one of Ocr polypeptide, ArdA polypeptide, NuiA polypeptide, HI1450 polypeptide, DMP12 polypeptide, MfpA polypeptide, Arn polypeptide, Gam polypeptide and variants thereof.

12. The nucleic acid expression system of claim 1, wherein the helper polypeptide is or comprises a modulator of innate immunity, optionally wherein the modulator of innate immunity is selected from any one of viral interferon regulatory factor 1 (vIRF1) polypeptide, ORF52/KicGAS polypeptide, PLP2-TM polypeptide, PLP2 polypeptide, US11 polypeptide, and variants thereof.

13. The nucleic acid expression system of claim 1, wherein the helper polypeptide is or comprises a synthetic cell surface receptor polypeptide, optionally wherein the synthetic cell surface receptor polypeptide is selected from any one of TVA-EGF polypeptide, H-EGF polypeptide, H-IGF1 polypeptide, and variants thereof.

14. A composition comprising the nucleic acid expression system of claim 1.

15. The composition of claim 14, wherein the composition is a pharmaceutical composition.

16. A pharmaceutical composition comprising the nucleic acid expression system of claim 1, and a pharmaceutically acceptable carrier.

17. A cell comprising the nucleic acid expression system of claim 1.

18. A method comprising:
contacting a target cell with an oligonucleotide comprising a payload sequence; and
contacting the target cell with at least one oligonucleotide comprising a sequence that encodes a helper polypeptide.

19. The method of claim 18, wherein the helper polypeptide is selected from the following: a nuclear localization signal (NLS) polypeptide, a DNA mimic polypeptide, a modulator of innate immunity, and a synthetic cell surface receptor polypeptide.

20. The method of claim 18, wherein the target cell is contacted with the oligonucleotide comprising a payload sequence and the at least one oligonucleotide comprising a sequence that encodes a helper polypeptide separately (e.g., in a sequential manner).

21. The method of claim 18, wherein the target cell is contacted with the oligonucleotide comprising a payload sequence and the at least one oligonucleotide comprising a sequence that encodes a helper polypeptide concurrently.

22. The method of claim 18, wherein the method is for at least one of the following:
(a) enhancing expression and/or activity of an oligonucleotide comprising a payload sequence in a target cell;
(b) enhancing nuclear import of an oligonucleotide comprising a payload sequence in a target cell;
(c) enhancing persistence or uptake of an oligonucleotide comprising a payload sequence in a target cell;
(d) enhancing the viability of a target cell upon contacting with an oligonucleotide comprising a payload sequence; and
(e) reducing non-specific toxicity induced in a target cell by an oligonucleotide comprising a payload sequence.
